SEQUENCE LISTING (2 sequences)

SEQ ID NO: 1 - protein, 313 aa, Homo sapiens
MLLSKINSLAHLRAAPCNDLHATKLAPGKEKEPLESQYQVGPLLGSGGFGSVYSGIRVSDNLPVAIKHVEKDRISDWGELPNGTRVPMEVVLLKKVSSGFSGVIRLLDWFERPDSFVLILERPEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIRGQVFFRQRVSSECQHLIRWCLALRPSDRPTFEEIQNHPWMQDVLLPQETAEIHLHSLSPGPSK

SEQ ID NO: 2 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

(19) United States
(12) Patent Application Publication
(10) Pub. No.: US 2008/0254481 A1
(43) Pub. Date: Oct. 16, 2008

(54) METHODS AND KITS FOR DETECTING PROSTATE CANCER BIOMARKERS

(75) Inventors: Bradley Love, Timonium, MD (US); Jeffrey Rogers, Escondido, CA (US); Joseph Beechem, Eugene, OR (US); Lilin Wang, San Diego, CA (US)

Correspondence Address: GREENLEE WINNER AND SULLIVAN P C, 4875 PEARL EAST CIRCLE, SUITE 200, BOULDER, CO, 80301, US

(73) Assignee: INVITROGEN CORPORATION, Carlsbad, CA (US)

(21) Appl. No.: 11/939,484

(22) Filed: Nov. 13, 2007

Related U.S. Application Data
(60) Provisional application No. 60/865,621, filed on Nov. 13, 2006.

(51) Int. Cl. G01N 33/53; G01N 33/566; B01J 19/00

(52) U.S. Cl. 435/7.1; 436/501; 422/68.1

(57) ABSTRACT

Provided herein are novel autoantibody biomarkers, and panels for detecting autoantibody biomarkers for prostate cancer, and methods and kits for detecting these biomarkers in the serum of individuals suspected of having prostate cancer.

### CROSS REFERENCE TO RELATED APPLICATIONS

This application claims benefit of U.S. Provisional Application No. 60/865,621, filed Nov. 13, 2006, which is incorporated by reference in its entirety herein to the extent that there is no consistency with the present disclosure.

### BACKGROUND

The invention generally relates to biomarkers associated with prostate cancer, and methods and compositions for the detection, diagnosis, prognosis, and monitoring of the progression of prostate cancer.

Prostate cancer (also referred to herein as “PCa”) is the most prevalent form of cancer and the second most common cause of cancer death in American men (Jemal et al. (2007) “Cancer statistics,” CA Cancer J. Clin. 57(1):43-66). When prostate cancer is diagnosed in its early stages, however, the prognosis is very good, with a ten year survival rate of greater than 85%. Current treatment modalities include radiation therapy, surgery, and androgen deprivation therapy. Treatment of prostate cancer can have serious side effects, including impairment of sexual or urinary function, thus the decision to intervene should be made on the most reliable criteria possible.

Accurate, early diagnosis of prostate cancer has proven challenging however, as the current diagnostic test for prostate cancer relies on detection of prostate-specific antigen (PSA) levels, an indicator that also correlates with benign prostate hypertrophy (BPH), a non-life threatening condition that does not increase cancer risk. BPH is found in about half of men at age 60, and about 90% of men reaching the age of 85. The PSA test is currently widely used in prostate cancer diagnosis. In general, a blood serum level of 4 ng per ml or higher of PSA is considered suggestive of prostate cancer, while a PSA level of 10 ng per ml or higher is considered highly suggestive of prostate cancer. While the PSA test has a fairly good sensitivity (80%), it suffers from a false positive rate that approaches 75%. It is estimated that for PSA values of 4-10 ng/mL, only one true diagnosis of prostate cancer was found in approximately 4 biopsies performed (Catalona et al. (1994) “Comparison of digital rectal examination and serum prostate specific antigen in the early detection of prostate cancer: results of a multicenter clinical trial of 6,630 men,” J. Urol. 151(5):1283-90). Tests that measure the ratio of free to total (free plus bound) PSA do not have significantly greater specificity or sensitivity than the standard PSA test.

Recently a urinary test has been developed based on detection of the PCA3 transcript. However, the reliability of the test depends on its being performed in conjunction with an attentive digital rectal exam (DRE), which means the time and effort of a trained clinician are required, as well as the willingness of the patient to undergo DRE.

Another condition known as prostate intraepithelial neoplasia (PIN) may precede prostate cancer by five to ten years, but requires no treatment or intervention. Currently there are no specific diagnostic tests for PIN, although the ability to detect and monitor the potentially pre-cancerous condition would contribute to early detection and enhanced survival rates for prostate cancer.

Autoantibody-based approaches using protein microarrays have a number of distinct advantages for the discovery of quality diagnostic biomarkers. Many of the potentially best disease-based biomarkers are not secreted into the blood at levels that are detectable in a robust manner. Such biomarkers will always be “unavailable” for convenient in-vitro blood-based diagnostic tests. However, autoantibodies to these same specific non-secreted biomarkers (if formed), will circulate beyond the boundaries of the diseased tissue and will be stable in whole-blood for extended periods of time. Using a protein array approach one can very quickly explore of a large number of potential protein targets for the presence of autoantibodies to correlate with specific diseases.

Cancer initiation and progression has been shown to associate with the process of immunoediting (Dunn et al. (2004), “The three Es of cancer immunoediting,” Annu Rev Immunol. 22:329-60). In immunoediting, the immune system interacts with cancer and induces a cancer specific immune response, with unique immune signatures characteristic of the various stages of cancer progression. Tumor associated antigens including peptides, proteins and polysaccarides have been utilized in microarray or ELISA experiments to profile cancer and normal sera.

Prostate cancer progression involves multiple steps including: prostatic intraepithelial neoplasia (PIN), localized carcinoma, invasive carcinoma and metastasis. The genetic and epigenetic events in prostate tumorigenesis include the loss of function of tumor suppressors, cell cycle and apoptosis regulators, proteins in metabolism machinery and stress response, angiogenesis and metastasis related molecules (Abate-Shen et al. (2000) “Molecular genetics of prostate cancer,” Genes Dev. 14(19):2410-34; Ciocca et al. (2005) “Heat shock proteins in cancer: diagnostic, prognostic, predictive, and treatment implications,” Cell Stress Chaperones 10(2):86-103). These proteins could potentially serve as PCa antigens and induce autoantibody response in PCa patients. Furthermore these induced autoantibodies can be used for PCa diagnosis either in a single- or multiple-marker format. While PCa results from the deregulated proliferation of epithelial cells, BPH majorly results from normal epithelial cell proliferation which does not frequently lead to malignancy (Ziada et al. (1999) “Benign prostatic hyperplasia: an overview,” Urology 53(3 Suppl 3D):1-6). Immune profiling using serum samples from PCa and BPH patients will help to identify biomarkers with unique autoantibody patterns in PCa, clearly distinguishable from the BPH autoantibody signature(s).

### SUMMARY OF THE INVENTION

The invention relates generally to the detection of autoantibodies related to cancer, and more particularly to methods of diagnosing, prognosing, and monitoring prostate cancer using panels of antigens for the detection of autoantibodies.

The invention recognizes the need for an accurate test for prostate cancer, and in particular for a minimally invasive test that can detect prostate cancer and, preferably, distinguish prostate cancer from benign prostate hypertrophy (BPH) with high sensitivity and specificity. The invention is based in part on a collection of target antigens and target antibodies for detecting autoantibodies, and on the detection of autoantibody biomarkers for the detection, diagnosis, prognosis, staging, and monitoring of cancer, particularly prostate cancer. The invention provides biomarkers and biomarker detection panels that include autoantigens, in which the biomarker detection panels have high selectivity and sensitivity for the detection of prostate cancer and for the diagnosis of prostate cancer over BPH. The invention also provides methods of detecting, diagnosing, prognosing, staging, and monitoring prostate cancer by detecting prostate cancer biomarkers in a test sample of an individual.

One aspect of the invention is a method of detecting an autoantibody in a sample from an individual. The method includes: contacting a sample from the individual with an autoantibody capture molecule of the invention, and detecting binding of an antibody in the sample to the autoantibody capture molecule, thereby detecting an autoantibody in the individual. The autoantibody capture molecule can be a target antigen that recognizes an autoantibody, or can be a target antibody that can bind an autoantigen complexed with an autoantibody. A target antigen can be an entire protein, such as the protein referred to as a target antigen, or a variant or modified form of the designated proteins, or a target antigen can be an epitope-containing fragment of the protein named as a target antigen. An autoantibody capture molecule that is a target antibody is an antibody that can bind an autoantigen that is complexed with an autoantibody. An autoantibody capture molecule can be, for example, any of the autoantibody capture molecules listed in Table 1 or Table 11a, or can be an antibody to any of the target antigens of Table 1 or Table 11a. In some embodiments, the autoantibody capture molecules are a target antigen of Table 2 or an antibody to any of the target antigens provided in Table 2, in which the antibody can specifically bind an autoantibody-autoantigen complex that includes a target antigen of Table 2.

In one embodiment, a sample from the individual is contacted with two or more autoantibody capture molecules, wherein the autoantibody capture molecules are autoantibody capture molecules of Table 1 or Table 11a, or are target antibodies against an antigen of Table 1 or Table 11a. In further embodiments, the autoantibody capture molecules are autoantibody capture molecules of Table 2, Table 3, Table 4, Table 10, or target antibodies to antigens in these tables (which can be described as subsets of Table 1). An autoantibody, which may correlate to the diagnosis of prostate cancer, is detected in the sample when an antibody or antibody-containing complex is detected to have bound to at least two of the autoantibody capture molecules. Preferably, the binding of the autoantibody capture molecules to antibodies or antibody-containing complexes in the test sample distinguishes between prostate cancer and BPH, and preferably distinguishes between Low Grade prostate cancer and High Grade prostate cancer.

In some embodiments of the present invention, the methods of detecting and diagnosing PCa and the biomarker detection panels exclude autoantibody capture molecules of PSA.

In a further embodiment, the sample from the individual is contacted with two or more autoantibody capture molecules, in which at least one of the autoantibody capture molecules is selected from the group consisting of KDR, PIM-1, LGALS8, GDF15, RPL23, RPL30, SFRP4, QSCN6, NCAM2, HOXB13, SH3GLB1, CLDN3, CLDN4, PTEN, CCNB1, AMACR, TP53, MUC1, KLK3, BIRC5, and target antibodies to KDR, PIM-1, LGALS8, GDF15, RPL23, RPL30, SFRP4, QSCN6, NCAM2, HOXB13, SH3GLB1, CLDN3, CLDN4, PTEN, CCNB1, AMACR, TP53, MUC1, KLK3, and BIRC5. In some preferred embodiments, the sample is contacted with KDR and/or PIM-1. It is understood that “KDR and/or PIM-1” encompasses full length KDR, a variant of KDR recognized by an antibody that recognizes KDR, a fragment of KDR comprising an epitope recognizable by an antibody, and/or full length PIM-1, a variant of PIM-1 recognized by an antibody that recognizes PIM-1, and a fragment of PIM-1 comprising an epitope recognizable by an antibody.

In certain aspects, a biomarker panel of the present invention includes a first biomarker that includes an epitope of KDR and a second biomarker that includes an epitope of PIM-1. In certain illustrative embodiments, the epitope of the first biomarker and/or the second biomarker is an epitope that is known to be recognized by autoantibodies in the sera of human subjects. In certain embodiments, the first biomarker and/or the second biomarker are at least 5 kDa or at least 10 kDa. In an illustrative embodiment the first biomarker is full-length PIM-1 and the second biomarker is full-length KDR.

In another aspect of the invention, any of fourteen novel tumor antigens (or variants or fragments thereof), or antibodies to these antigens, that have not been previously reported as inducing an autoantibody response are contacted with a sample to detect prostate cancer and to distinguish prostate cancer from BPH. In one embodiment, a sample from an individual suspected as having prostate cancer is contacted with one or more autoantibody capture molecules are selected from the group consisting of KDR, PIM-1, LGALS8, GDF15, RPL23, RPL30, SFRP4, QSCN6, NCAM2, HOXB13, SH3GLB1, CLDN3, CLDN4, PTEN, and target antibodies to KDR, PIM-1, LGALS8, GDF15, RPL23, RPL30, SFRP4, QSCN6, NCAM2, HOXB13, SH3GLB1, CLDN3, CLDN4, and PTEN. Preferably, the sample is contacted with one or more autoantibody capture molecules including KDR and/or PIM-1. It is understood that these terms encompasses full length KDR, a variant of KDR recognized by an antibody that recognizes KDR, a fragment of KDR comprising an epitope recognizable by an antibody, full length PIM-1, a variant of PIM-1 recognized by an antibody that recognizes PIM-1, and a fragment of PIM-1 comprising an epitope recognizable by an antibody.

The assays suitable for use with the present invention includes assays used to detect autoantibodies in fluid samples from individual, as well non-fluid samples, such as a prostate tissue sample, from an individual. The sample used in the assays and detection and diagnosis methods of the invention can be any type of sample, but preferably is a saliva sample or a blood sample, or a fraction thereof, such as plasma or serum. In some embodiments, the sample is blood or a fraction thereof, such as, for example, serum. In other embodiments, the sample is a non-fluid sample such as a tissue sample. In a further embodiment, the tissue sample is a prostate tissue sample. The individual can be an individual that is being screened for cancer, and in some embodiments is a male individual being screened for prostate cancer.

In some embodiments, the methods are directed to detecting prostate cancer, in which the methods include: determining the immune reactivity of a test sample from the individual against an autoantibody capture molecule, in which the autoantibody capture molecule is one of molecules of Table 4 (or a variant or fragment thereof), or an antibody to any of the target antigens of Table 4, in which the antibody can specifically bind an autoantibody-autoantigen complex, and correlating the immune reactivity of the test sample to the capture molecule to a diagnosis of prostate cancer. The method can in some embodiments be used to distinguish prostate cancer from BPH.

In another aspect, the invention provides methods of diagnosing prostate cancer in an individual by contacting a sample from an individual with a biomarker detection panel comprising two or more autoantibody capture molecules of Table 1, or an antibody to any of the target antigens of Table 1; and detecting the pattern of immune reactivity of the test sample to the biomarker detection panel, in which the pattern of immune reactivity of the test sample to the biomarker detection panel is indicative of the presence of prostate cancer. An autoantibody capture molecule can be a target antibody or a target antigen. A target antigen can be an entire protein, such as a protein referred to as a target antigen, or can be a variant, processed, unprocessed, or modified form of the designated protein, or can be or comprise an epitope-containing fragment of the protein designated. An autoantibody capture molecule that is a target antibody is an antibody that can detect an autoantibody in a sample that is complexed to an autoantigen.

A biomarker detection panel used in the methods of the invention comprises a plurality of autoantibody capture molecules, for example, 2 or more, 3 or more, 4 or more, 5 or more, 6 or more, 7 or more, 10 or more, 20 or more, 50 or more, 100 or more, 200 or more, 500 or more, 1,000 or more, 2,000 or more, 5,000 or more, or 10,000 or more, of which from 2 to 214 of the autoantibody capture molecules are from Table 1 and/or Table 11a.

An autoantibody capture molecule included in the present invention, in certain embodiments is at least 70%, 75%, 80%, 85%, 90%, 95%, or 100% identical to an at least 25, 50, 75, 100 or the entire amino acid segment of SEQ ID NO:1 or SEQ ID NO:2. The autoantibody capture molecule in certain illustrative embodiments binds to an autoantibody of KDR or PIM-1.

In preferred embodiments, at least one of the autoantibody capture molecules of the biomarker detection panel is an autoantibody capture molecule of Table 3. The biomarker detection panel used to detect prostate cancer can in some embodiments comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, between 30 and 35, between 35 and 40, between 40 and 45, between 45 and 50, between 50 and 55, between 55 and 60, between 60 and 65, or between 65 and 70 autoantibody capture molecules of Table 3 or antibodies to any of the antigens of Table 3, in which antibodies to the antigens of Table 3 used on the chip are used for detection of autoantibodies via binding of autoantigen-autoantibody complexes of the sample.

In some embodiments, the biomarker detection panel comprises at least two autoantibody capture molecules of Table 3. In some embodiments, the biomarker detection panel comprising two or more autoantibody capture molecules of Table 1 includes at least one target antigen selected from the group consisting of Table 4. In some preferred embodiments, the biomarker detection panel used in the methods of the invention has an ROC curve with an AUC value (also referred to as ROC/AUC value) for distinguishing prostate cancer from BPH is 0.800 or greater. In some preferred embodiments of the method, the ROC curve with an AUC value of the biomarker detection panel for distinguishing the presence of PCa from BPH is 0.900 or greater.

In some embodiments the invention includes methods of diagnosing prostate cancer and methods of distinguishing prostate cancer from BPH that include: contacting a sample from an individual with a biomarker detection panel that includes two or more autoantibody capture molecules of Table 3, in which at least one of the autoantibody capture molecules is from Table 10, and detecting the pattern of immune reactivity of the sample to the biomarker detection panel, in which the pattern of immune reactivity of the sample to the biomarker detection panel distinguishes prostate cancer from BPH in the individual. In some exemplary embodiments, the biomarker detection panel comprises at least one 3-marker autoantibody detection set of Table 5, at least one 4-marker autoantibody detection set of Table 6, at least one 5-marker autoantibody detection set of Table 7, at least one 6-marker autoantibody detection set of Table 8, at least one 7-marker autoantibody detection set of Table 9.

The biomarker detection panel in some embodiments has a specificity of 80% or greater, 85% or greater, 90% or greater, 96% or greater, or 98% or greater, and/or a sensitivity of 80% or greater, 90% or greater, 96% or greater, 98% or greater, or 100%, for diagnosing prostate cancer, or for discriminating prostate cancer from BPH in an individual. The biomarker detection panel in some embodiments has a Bayesian specificity of 78% or greater, 85% or greater, or 90% or greater, for diagnosing prostate cancer, and/or a Bayesian sensitivity of 80% or greater, 90% or greater, or 95% or greater for diagnosing prostate cancer, or for discriminating prostate cancer from BPH in an individual. The biomarker detection panel in some exemplary embodiments has a Bayesian accuracy of 80% or greater, 85% or greater, 85% or greater, 90% or greater, or 96% or greater for diagnosing prostate cancer, or for discriminating prostate cancer from BPH in an individual.

In preferred embodiments of the methods for diagnosing prostate cancer, the test sample is blood or a fraction thereof, such as serum. In some embodiments, the individual is a male aged 50 or older. In some embodiments, the method is repeated over time for the individual. In some embodiments, the individual is monitored at regular or irregular intervals after cancer treatment by determining immune reactivity of samples of the patient to a biomarker detection panel of the invention. The immune reactivity of a sample tested at a later date can be compared with the immune reactivity of a sample taken at an earlier date.

The invention provides in yet other aspects biomarker detection panels for diagnosing, prognosing, monitoring, or staging prostate cancer, or distinguishing prostate cancer from BPH, that include two or more autoantibody capture molecules of Table 1, in which at least one of the antibody capture molecules is of Table 3. A biomarker detection panel of the invention comprises a plurality of autoantibody capture molecules, for example, 2 or more, 3 or more, 4 or more, 5 or more, 6 or more, 7 or more, 10 or more, 20 or more, 50 or more, 100 or more, 200 or more, 500 or more, or 1,000 or more autoantibody capture molecules. The biomarker detection panel can comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, between 30 and 35, between 35 and 40, between 40 and 45, between 45 and 50, between 50 and 55, between 55 and 60, between 60 and 65, or between 65 and 70 autoantibody capture molecules of Table 3.

In some preferred embodiments, a biomarker detection panel includes at least one of the autoantibody capture molecules selected from Table 2. In some preferred embodiments, a biomarker detection panel includes at least one of the autoantibody capture molecules selected from Table 4. The invention provides biomarker detection panels that include KDR, PIM-1, or both KDR and PIM-1.

In some embodiments a biomarker detection panel can further comprise antibodies such as but not limited to one or more of antibodies to ACCP, BCL2, PSA (total), PSA (free), CXCR4, PTGER2, IL-6, IL-8, PAP, or PSMA. In some preferred embodiments, biomarker detection panel comprises antibodies to ACCP and/or IL-6.

In some preferred embodiments of biomarker detection panels, at least one of the autoantibody capture molecules is selected from Table 10. In some exemplary embodiments, a biomarker detection panel of the invention comprises one or more autoantibody detection sets of Table 5, Table 6, Table 7, Table 8, or Table 9.

In some preferred embodiments, the biomarker detection panel is provided bound to one or more solid or semi-solid supports, such as, for example, a gel or matrix, beads, particles, fibers, rods, filaments, or a filter, strip, sheet, membrane, plate (for example, a multiwell plate), dish, chip or array. In some preferred embodiments, at least 50% of the human proteins bound to the solid support are test antigens of the biomarker detection panel. In some preferred embodiments, at least 55%, at least 60%, at least 65%, at least 70%, at least 75%, at least 80%, at least 85%, at least 90%, or at least 95% of the human proteins bound to the solid support are test antigens of the biomarker detection panel. In some preferred embodiments, the biomarker detection panel is provided in or on a protein array.

The invention also provides kits that include one or more biomarker detection panels as provided herein. The kits can include one or more reagents for detecting binding of an antibody, or an antigen-antibody complex, from a sample. Detection reagents can include one or more antibodies, labels, labeling reagents, or buffers. In some embodiments, the one or more autoantibody capture molecules of a biomarker panel of a kit are provided bound to a solid support. In some embodiments of kits, the kit provides a biomarker detection panel in which the target antigens of the detection panel are bound to a chip or array.

A kit of the present invention can include 2 or more autoantibody capture molecules of Table 1 or Table 11a associated with different vessels and/or solid supports.

### BRIEF DESCRIPTION OF THE DRAWINGS

FIG. 1 is a depiction of an autoantibody chip of the present invention that was used to identify prostate cancer autoantibody biomarkers. The array on the chip comprises 8 capture antibodies, 108 auto-antigens, mouse anti-human K 3-step (positive control), mouse anti-human IgG1 3-step (positive control), protein L 3-step (positive control), human IgG 4-step (positive control), and 1137 empty spots. The array shown in FIG. 1 is printed in duplicate on each chip, so every spot on the chip is replicated a total of four times.

FIGS. 2A-2D illustrate an autoantibody profiling experiment with pooled serum samples from 32 PCa and 32 BPH patients. The protein array is made of 96 protein antigens and the signal normalized using a protein L spot printed on the microarray. FIG. 2A is a representative image to show the difference between PCa and BPH. Among 90 antigens printed on the cellulose slides about half show significant autoantibody signals. The arrows show KDR and PIM-1 spots which capture significantly higher autoantibody signals in pooled PCa serum than in pooled BPH serum. FIG. 2B shows the top 20 protein antigens showing the highest fold difference between PCa serum and BPH serum. FIG. 2C shows an autoantigen competition experiment using purified KDR protein at the concentration as shown. At 1.8 ug/ml concentration, half of the KDR signals were eliminated. Similarly, FIG. 2D shows an autoantigen competition experiment using purified PIM-1 protein at the concentrations as shown. At 2 ug/ml concentration half of the PIM-1 signal was eliminated.

FIGS. 3A-3C illustrate autoantibody fluorescence signal profiles of 32 PCa patients (numbered 1-32) and 32 BPH patients (numbered 33-64) for both KDR antigen (FIG. 3A) and PIM-1 antigen (FIG. 3B). Only the odd patient numbers are labeled in the figures. The signal threshold level determined by ROC analysis is denoted by horizontal dashed line. FIG. 3C shows a plot of ROC curves of 64 sera data set using 1-plex analysis of KDR (green), PIM-1 (red), PSA (dark blue) and a 2-plex analysis of KDR & PIM-1 (light blue) combination. KDR and PIM-1 2-plex analysis generates a sensitivity and specificity of 90.6% and 84.4% respectively. AUCs for all analyses are shown in the legend. The experiments were done with a low content microarray containing KDR and PIM-1 antigens.

FIG. 4 shows images of prostate tissue microarray experiments with anti-KDR and anti-PIM-1 antibodies. Red fluorescence shows the autoantibody signals detected by Alexa 647 labeled goat anti-human IgG and the blue fluorescence indicated the counter-staining of nuclei by DAPI. The images show that KDR and PIM-1 are preferentially expressed in PCa tissues. Over expression of KDR and PIM-1 proteins lead to the aberrant humoral response in PCa patients.

FIG. 5 shows the scheme for the autoantibody profiling experiment described in FIGS. 2A-2D. Samples from individuals having prostate cancer and BPH were collected and contacted with a chip containing possible target antigens selected based on their role in prostate cancer. The resulting binding of target antigens to autoantibodies was quantified and used to identify biomarkers selective for prostate cancer over BPH.

### DETAILED DESCRIPTION

The invention is based on the identification of candidate antigens for the detection of autoantibodies in samples from individuals. Test antigens and test antibodies provided in Table 1 and are human proteins selected based on knowledge of prostate cancer biology. Of the test antigens listed in Table 1, TP53, PTEN, PDLIM1,SPRX, NUCB1, and PSCA are prostate cancer pathway-specific tumor suppressor genes; FOLH1, KDR, PSIP1, EGFR, ERBB2, CCKBR, XLKD1, MMP9, TMPRSS2, AGR2, PRSS8, MUC1, LGALS8, CD164, CXCR4, NRP1, STEAP1, HPN, MET, PTGER3, CLDN3, CLDN4, NCAM2, EDNRB, FLT1, PECAM1, BDKRB2, CD151, QSCN6, ERG, PCNA, EPCAM, and MAD1L1 are cell surface proteins expressed by some cancer cells; HSPA1A, HSPB1, SERPINH1, HSPA5, TRA1, MICB, PSMAB4, UBE2C, STIP1, HSPD1, and UBQLN1 are proteins involved in innate immunity; EIF4G1 ALOX15, PTGS1, RPL23, RPS14, ELAC1, EIF3S3, TOP2A, RPS6KA1, ACPP, KLK3, FASN, RPL30, and ENO1 are proteins involved in cell metabolism; CCNB1, CCND1, CCNA, CDKN2A, CUL4A, BIRC5, MYC, ETS2, BCL2, BCLG, TP53BP2, GDF15, RASSF1, AKT1, MDM2, PIM1, SH3GLB1, HIMP2, HIMP3, KHDRBS1, PCNA, and CAV3, are cell cycle or apoptosis-related proteins; and E6 and E7 are human papillomavirus antigens. HIP1, BRD2, AZGP1, COVA1, MLH1, TPD52, PSAP, MIB1, HOXB13, RDH11, HMGA2, ZWINT, RCV1, SFRP4, SPRR1B, HMGA2, HIP2, and HEYL were also found to be cancer-associated.

TABLE 1
Test Antigens and Test Antibodies

| Marker | | | | |
|---|---|---|---|---|
| (Autoantibody | | | | |
| Capture | HUGO | Protein/Gene | Genbank | |
| Molecule) | designation | Aliases | Identifiers | Source |
| ABV0G41VX- | KLK3 | KLK3, APS, PSA, | kallikrein 3, (prostate | In vitro |
| KLK3 | | hK3, KLK2A1 | specific antigen) | synthesized |
| | | | GI: 22208991 | wheat germ |
| | | | NM_145864 | (WG IVT) - |
| | | | | Abnova; Tapei |
| | | | | City, Taiwan |
| ACPP | ACPP | ACPP, PAP, ACP3, | acid phosphatase, | WG IVT |
| | | ACP-3 | prostate | |
| | | | GI: 6382063 | |
| | | | NM_001099 | |
| AGR2 | AGR2 | AGR2, AG2, GOB- | anterior gradient 2 | WG IVT |
| | | 4, HAG-2, XAG-2 | homolog (Xenopus | |
| | | | laevis) | |
| | | | GI: 20070225NM_006408 | |
| AKT1 | AKT1 | AKT1, PKB, RAC, | v-akt murine thymoma | WG IVT |
| | | PRKBA, | viral oncogene homolog 1 | |
| | | MGC99656, RAC- | GI: 62241010 | |
| | | ALPHA | NM_005163 | |
| ALOX15 | ALOX15 | ALOX15 | arachidonate 15- | WG IVT |
| | | | lipoxygenase | |
| | | | GI: 40316936 | |
| | | | NM_001140 | |
| AMACR | AMACR | AMACR, RACE | alpha-methylacyl-CoA | WG IVT |
| | | | racemase | |
| | | | NM_014324 | |
| | | | GI: 42794624 | |
| anti-ACPP | (antibody) | anti-PAP mouse | | United Biotech |
| | | capture mAb | | |
| anti-BCL2 | (antibody) | mouse anti-bcl-2 | | Zymed |
| anti-CXCR4 | (antibody) | mouse anti-CXCR4 | | Zymed |
| | | monoclonal | | |
| anti-IL6 | (antibody) | cap Ab from | | Biosource |
| | | cytosets assay kit | | |
| | | for hIL-6 58.126.09 | | |
| | | mu clone 677B 6A2 | | |
| | | IgG1 | | |
| anti-IL8 | (antibody) | capture Ab from IL8 | | Biosource |
| | | Cytosets Kit | | |
| anti-PSA(f) | (antibody) | (Free PSA coat Ab) | | Biospacific |
| anti-PSA(t) | (antibody) | (Total PSA coat Ab) | | Medix |
| anti-PTER2 | (antibody) | mouse anti-PTER2 | | GeneTex |
| | | monoclonal | | |
| AZGP1 | AZGP1 | AZGP1, ZAG, | alpha-2-glycoprotein 1, | WG IVT |
| | | ZA2G | zinc | |
| | | | GI: 38372939 | |
| | | | NM_001185 | |
| BCL2 | BCL2 | BCL2, Bcl-2 | B-cell CLL/lymphoma 2 | WG IVT |
| | | | (BCL2), nuclear gene | |
| | | | encoding | |
| | | | mitochondrial | |
| | | | protein | |
| | | | GI: 72198188 | |
| | | | NM_000633 | |
| BCLG | BCL2L14 | BCL2L14, BCLG | BCL2-like 14 (apoptosis | WG IVT |
| | | | facilitator) GI: 13540528 | |
| | | | NM_030766 | |
| BDKRB2 | BDKRB2 | BDKRB2, B2R, | bradykinin receptor B2 | WG IVT |
| | | BK2, BK-2, BKR2, | GI: 17352499 | |
| | | BRB2, | NM_000623 | |
| | | DKFZp686O088 | | |
| BIRC5 | BIRC5 | BIRC5, API4, EPR-1 | baculoviral IAP repeat- | WG IVT |
| | | | containing 5 (survivin) | |
| | | | GI: 59859879 | |
| | | | NM_001012270 | |
| BRD2 | BRD2 | BRD2, NAT, RNF3, | bromodomain containing | WG IVT |
| | | FSRG1, RING3, | 2 GI: 12408641 | |
| | | D6S113E, | NM_005104 | |
| | | FLJ31942, | | |
| | | KIAA9001, | | |
| | | DKFZp686N0336 | | |
| CAV3 | CAV3 | CAV3, VIP21, | caveolin 1, caveolae | WG IVT |
| | | LGMD1C, VIP-21, | protein, 22 kDa | |
| | | MGC126100, | GI: 15451855 | |
| | | MGC126101, | NM_001753 | |
| | | MGC126129 | | |
| CCKBR | CCKBR | CCKBR, GASR, | cholecystokinin B | Purified from |
| | | CCK-B | receptor | human serum, |
| | | | GI: 33356159 | (EMD |
| | | | NM_176875 | Biosciences, |
| | | | | San Diego, |
| | | | | CA) |
| CCNA1 | CCNA1 | CCNA1 | cyclin A1 | WG IVT |
| | | | GI: 16306528 | |
| | | | NM_003914 | |
| CCNB1 | CCNB1 | CCNB1, CCNB | cyclin B1 | WG IVT |
| | | | GI: 34304372 | |
| | | | NM_031966 | |
| CCND1 | CCND1 | CCND1, BCL1, | cyclin D1 | WG IVT |
| | | PRAD1, U21B31, | GI: 77628152 | |
| | | D11S287E | NM_053056 | |
| CD151 | CD151 | CD151, GP27, | CD151 molecule (Raph | WG IVT |
| | | MER2, RAPH, | blood group) | |
| | | SFA1, PETA-3, | GI: 87159810 | |
| | | TSPAN24 | NM_004357 | |
| CD164 | CD164 | CD164, MGC-24, | CD164 molecule, | WG IVT |
| | | MUC-24, endolyn | sialomucin GI: 34222157 | |
| | | | NM_006016 | |
| CDKN2A | CDKN2A | CDKN2A, ARF, | cyclin-dependent kinase | WG IVT |
| | | MLM, p14, p16, | inhibitor 2A GI: 47132606 | |
| | | p19, CMM2, INK4, | NM_000077 | |
| | | MTS1, TP16, | | |
| | | CDK4I, CDKN2, | | |
| | | INK4a, p14ARF, | | |
| | | p16INK4, | | |
| | | p16INK4a | | |
| CLDN3 | CLDN3 | CLDN3, RVP1, | claudin 3 | WG IVT |
| | | HRVP1, C7orf1, | GI: 21536298 | |
| | | CPE-R2, CPETR2 | NM_001306 | |
| CLDN4 | CLDN4 | CLDN4, CPER, | Claudin 4 | WG IVT |
| | | CPE-R, CPETR, | GI: 34335232 | |
| | | CPETR1, | NM_001305 | |
| | | WBSCR8, hCPE-R | | |
| COVA1 | COVA1 | COVA1, APK1, | cytosolic ovarian | WG IVT |
| | | tNOX | carcinoma antigen 1 | |
| | | | GI: 32528292 | |
| | | | NM_006375 | |
| CUL4A | CUL4A | CUL4A | cullin 4A (CUL4A), | WG IVT |
| | | | transcript variant 2 | |
| | | | GI: 57165422 | |
| | | | NM_003589 | |
| CXCR4 | CXCR4 | CXCR4, FB22, | chemokine (C—X—C motif) | WG IVT |
| | | HM89, LAP3, | receptor 4 GI: 56790926 | |
| | | LCR1, NPYR, | NM_001008540 | |
| | | WHIM, CD184, | | |
| | | LESTR, NPY3R, | | |
| | | NPYRL, HSY3RR, | | |
| | | NPYY3R, D2S201E | | |
| E6 | NA | | HPV (viral) protein | WG IVT |
| E7 | NA | | HPV (viral) protein | WG IVT |
| EDNRB | EDNRB | EDNRB, ETB, | endothelin receptor type B | WG IVT |
| | | ETRB, HSCR, | GI: 4557546 | |
| | | ABCDS, HSCR2 | NM_000115 | |
| EGFR | EGFR | EGFR, ERBB, | epidermal growth factor | WG IVT |
| | | mENA, ERBB1 | receptor GI: 41327737 | |
| | | | NM_005228 | |
| EIF3S3 | EIF3S3 | EIF3S3, eIF3-p40, | eukaryotic translation | WG IVT |
| | | MGC102958, eIF3- | initiation factor 3, subunit 3 | |
| | | gamma | gamma, 40 kDa | |
| | | | GI: 83656776 | |
| | | | NM_003756 | |
| EIF4G1 | EIF4G1 | EIF4G1, p220, | eukaryotic translation | WG IVT |
| | | EIF4F, EIF4G, | initiation factor 4 gamma, 1 | |
| | | DKFZp686A1451 | GI: 38201620 | |
| | | | NM_182917 | |
| ELAC1 | ELAC1 | ELAC1, D29 | elaC homolog 1 (E. coli) | WG IVT |
| | | | GI: 50726987 | |
| | | | NM_018696 | |
| ENO1 | ENO1 | ENO1, NNE, PPH, | enolase 1, (alpha) | WG IVT |
| | | MPB1, MBP-1, | GI: 16507965 | |
| | | ENO1L1 | NM_001428 | |
| EP-CAM | TACSTD1 | TACSTD1, EGP, | tumor-associated | WG IVT |
| | | KSA, M4S1, MK-1, | calcium signal | |
| | | CD326, EGP40, | transducer 1 precursor | |
| | | MIC18, TROP1, | GI: 49457558 | |
| | | Ep-CAM, hEGP-2, | NM_002354 | |
| | | CO17-1A, GA733-2 | | |
| ERBB2 | ERBB2 | ERBB2, NEU, NGL, | v-erb-b2 erythroblastic | WG IVT |
| | | HER2, TKR1, HER- | leukemia viral oncogene | |
| | | 2, c-erb B2, HER- | homolog 2, | |
| | | 2/neu | neuro/glioblastoma | |
| | | | derived oncogene | |
| | | | homolog (avian) | |
| | | | GI: 54792097 | |
| | | | NM_001005862 | |
| ERG | ERG | ERG, p55, erg-3 | v-ets erythroblastosis | WG IVT |
| | | | virus E26 oncogene | |
| | | | homolog | |
| | | | (avian) | |
| | | | GI: 46255021 | |
| | | | NM_004449 | |
| ETS2 | ETS2 | ETS2 | epidermal growth factor | WG IVT |
| | | | receptor (erythroblastic | |
| | | | leukemia viral (v-erb-b) | |
| | | | oncogene homolog, | |
| | | | avian) | |
| | | | GI: 41327737 | |
| | | | NM_005228 | |
| FASN | FASN | FASN, FAS, OA- | fatty acid synthase | WG IVT |
| | | 519, MGC14367, | GI: 41872630 | |
| | | MGC15706 | NM_004104 | |
| FLT1 | FLT1 | FLT1, FLT, | fms-related tyrosine | WG IVT |
| | | VEGFR1 | kinase 1 (vascular | |
| | | | endothelial growth | |
| | | | factor/vascular | |
| | | | permeability factor | |
| | | | receptor) | |
| | | | GI: 32306519 | |
| | | | NM_002019 | |
| FOLH1 | FOLH1 | FOLH1, PSM, | folate hydrolase | WG IVT |
| | | FGCP, FOLH, | (prostate-specific | |
| | | GCP2, PSMA, | membrane antigen) | |
| | | mGCP, GCPII, | 1 GI: 4758397 | |
| | | NAALAD1, | NM_004476 | |
| | | NAALAdase | | |
| GDF15 | GDF15 | GDF15, PDF, | growth differentiation | WG IVT |
| | | MIC1, PLAB, MIC- | factor 15 GI: 4758935 | |
| | | 1, NAG-1, PTGFB, | NM_004864 | |
| | | GDF-15 | | |
| HEYL | HEYL | HEYL, HRT3, | hairy/enhancer-of-split | WG IVT |
| | | MGC12623 | related with YRPW motif- | |
| | | | like | |
| | | | GI: 105990530 | |
| | | | NM_014571 | |
| HIP1 | HIP1 | HIP1, ILWEQ, | huntingtin interacting | In vitro |
| | | MGC126506 | protein 1 | Synthesized |
| | | | GI: 38045918 | wheat germ |
| | | | NM_005338 | (Abnova; Tapei |
| | | | | City, Taiwan) |
| HIP2 | HIP2 | HIP2, LIG, HYPG, | huntingtin interacting | Synthesized in |
| | | UBE2K | protein 2 | E. coli (U of |
| | | | GI: 21536483 | MI) |
| | | | NM_005339 | |
| HMGA2 | HMGA2 | HMGA2, BABL, | high mobility group AT- | WG IVT |
| | | LIPO, HMGIC, | hook 2 GI: 62912481 | |
| | | HMGI-C | NM_003484 | |
| HOXB13 | HOXB13 | HOXB13, PSGD | homeobox B13 | WG IVT |
| | | | GI: 84043952 | |
| | | | NM_006361 | |
| HPN | HPN | HPN, TMPRSS1 | hepsin (transmembrane | WG IVT |
| | | | protease, serine 1) | |
| | | | GI: 4504480 | |
| | | | NM_002151 | |
| HSPA1A | HSPA1A | HSPA1A, HSP72, | heat shock 70 kDa | WG IVT |
| | | HSPA1, HSPA1B, | protein 1A GI: 26787973 | |
| | | HSP70-1 | NM_005345 | |
| HSPA5 | HSPA5 | HSPA5, BIP, MIF2, | heat shock 70 kDa | WG IVT |
| | | GRP78, FLJ26106 | protein 5 (glucose- | |
| | | | regulated protein, | |
| | | | 78 kDa) | |
| | | | GI: 21361242 | |
| | | | NM_005347 | |
| HSPB1 | HSPB1 | HSPB1, CMT2F, | heat shock 27 kDa | WG IVT |
| | | HSP27, HSP28, | protein 1 | |
| | | Hsp25, HS.76067, | GI: 4996892 | |
| | | DKFZp586P1322 | NM_001540 | |
| HSPD1 | HSPD1 | HSPD1, CPN60, | heat shock 60 kDa | WG IVT |
| | | GROEL, HSP60, | protein 1 (chaperonin) | |
| | | HSP65, SPG13, | GI: 41399283 | |
| | | HuCHA60 | NM_002156 | |
| IMP-2 | IGF2BP2 | IGF2BP2, p62, | insulin-like growth factor | WG IVT |
| | | IMP2, IMP-2, | 2 mRNA binding protein 2 | |
| | | VICKZ2 | GI: 64085376 | |
| | | | NM_006548 | |
| IMP-3 | IGF2BP3 | IGF2BP3, IMP3, | insulin-like growth factor | WG IVT |
| | | KOC1, IMP-3, | 2 mRNA binding protein 3 | |
| | | VICKZ3, | GI: 30795211 | |
| | | DKFZp686F1078 | NM_006547 | |
| KDR | KDR | KDR, FLK1, | kinase insert domain | WG IVT |
| | | CD309, VEGFR, | receptor (a type III | |
| | | VEGFR2 | receptor tyrosine kinase) | |
| | | | GI: 11321596 | |
| | | | NM_002253 | |
| KHDRBS1 | KHDRBS1 | KHDRBS1, p62, | KH domain containing, | WG IVT |
| | | Sam68 | RNA binding, signal | |
| | | | transduction associated 1 | |
| | | | GI: 5730026 | |
| | | | NM_006559 | |
| LGALS8 | LGALS8 | LGALS8, Gal-8, | lectin, galactoside- | WG IVT |
| | | PCTA1, PCTA-1, | binding, soluble, 8 | |
| | | Po66-CBP | GI: 42544184 | |
| | | | NM_006499 | |
| MAD1L1 | MAD1L1 | MAD1L1, MAD1, | MAD2 mitotic arrest | WG IVT |
| | | PIG9, HsMAD1, | deficient-like 1 | |
| | | TP53I9, TXBP181 | GI: 6466452 | |
| | | | NM_002358 | |
| MDM2 | MDM2 | MDM2, hdm2, | Mdm2, transformed 3T3 | WG IVT |
| | | MGC71221 | cell double minute 2, p53 | |
| | | | binding protein | |
| | | | GI: 46488903 | |
| | | | NM_002392 | |
| MET | MET | MET, HGFR, | met proto-oncogene | WG IVT |
| | | RCCP2 | (hepatocyte growth | |
| | | | factor receptor) | |
| | | | GI: 42741654 | |
| | | | NM_000245 | |
| MIB1 | MIB1 | MIB1, MIB, ZZZ6, | mindbomb homolog 1 | WG IVT |
| | | DIP-1, ZZANK2, | (Drosophila) | |
| | | FLJ90676, | GI: 62868229 | |
| | | MGC129659, | NM_020774 | |
| | | MGC129660, | | |
| | | DKFZp686I0769, | | |
| | | DKFZp761M1710 | | |
| MICB | MICB | MICB, PERB11.2 | MHC class I polypeptide- | WG IVT |
| | | | related sequence B | |
| | | | GI: 26787987 | |
| | | | NM_005931 | |
| MLH1 | MLH1 | MLH1, FCC2, | mutL homolog 1, colon | WG IVT |
| | | COCA2, HNPCC, | cancer, nonpolyposis | |
| | | hMLH1, HNPCC2, | type 2 | |
| | | MGC5172 | GI: 28559089 | |
| | | | NM_000249 | |
| MMP9 | MMP9 | MMP9, GELB, | matrix metallopeptidase 9 | WG IVT |
| | | CLG4B, MMP-9 | GI: 74272286 | |
| | | | NM_004994 | |
| MUC1 | MUC1 | MUC1, EMA, PEM, | mucin 1, cell surface | WG IVT |
| | | PUM, MAM6, | associated GI: 65301116 | |
| | | PEMT, CD227, | NM_002456 | |
| | | H23AG | | |
| MYC | MYC | MYC, c-Myc | v-myc myelocytomatosis | WG IVT |
| | | | viral oncogene homolog | |
| | | | (avian) | |
| | | | GI: 71774082 | |
| | | | NM_002467 | |
| NCAM2 | NCAM2 | NCAM2, NCAM21, | neural cell adhesion | WG IVT |
| | | MGC51008 | molecule 2 | |
| | | | GI: 33519480 | |
| | | | NM_004540 | |
| NRP1 | NRP1 | NRP1, NRP, | neuropilin 1 | WG IVT |
| | | CD304, | GI: 57162075 | |
| | | VEGF165R, | NM_015022 | |
| | | DKFZp781F1414, | | |
| | | DKFZp686A03134 | | |
| NUCB1 | NUCB1 | NUCB1, NUC, | nucleobindin 1 | WG IVT |
| | | FLJ40471, | GI: 39725676 | |
| | | DKFZp686A15286 | NM_006184 | |
| PCNA | PCNA | PCNA, MGC8367 | proliferating cell nuclear | WG IVT |
| | | | antigen GI: 33239450 | |
| | | | NM_182649 | |
| PDLIM1 | PDLIM1 | PDLIM1, CLIM1, | PDZ and LIM domain 1 | WG IVT |
| | | CLP36, ELFIN, | (elfin) | |
| | | CLP-36, hCLIM1 | GI: 20127594 | |
| | | | NM_020992 | |
| PECAM1 | PECAM1 | PECAM1, CD31, | platelet/endothelial cell | WG IVT |
| | | PECAM-1 | adhesion molecule | |
| | | | (CD31 antigen) | |
| | | | GI: 110347450 | |
| | | | NM_000442 | |
| PIM1 | PIM1 | PIM1, PIM | pim-1 oncogene | WG IVT |
| | | | GI: 31543400 | |
| | | | NM_002648 | |
| PRL | PRL | PRL | Prolactin | IV synthesized |
| | | | GI: 40254429 | in WG system |
| | | | NM_000948 | (Abnova, |
| | | | | Taipei City, |
| | | | | Taiwan) |
| PRL | PRL | PRL | Prolactin | Purified from |
| | | | GI: 40254429 | human serum |
| | | | NM_000948 | (Fitgerald |
| | | | | Industries |
| | | | | International, |
| | | | | Concord, MA) |
| PRSS8 | PRSS8 | PRSS8, CAP1, | Homo sapiens protease, | WG IVT |
| | | PROSTASIN | serine, 8 (prostasin) | |
| | | | GI: 21536453 | |
| | | | NM_002773 | |
| PSA | KLK3 | KLK3, APS, PSA, | kallikrein 3, (prostate | WG IVT |
| | | hK3, KLK2A1 | specific antigen) | |
| | | | GI: 22208991 | |
| | | | NM_145864 | |
| PSAP | PSAP | PSAP, GLBA, | prosaposin (variant | WG IVT |
| | | SAP1, FLJ00245, | Gaucher disease and | |
| | | MGC110993 | variant metachromatic | |
| | | | leukodystrophy) | |
| | | | GI: 110224477 | |
| | | | NM_002778 | |
| PSCA | PSCA | PSCA, PRO232 | prostate stem cell | WG IVT |
| | | | antigen GI: 83641882 | |
| | | | NM_005672 | |
| PSIP1 | PSIP1 | PSIP1, p52, p75, | PC4 and SFRS1 | WG IVT |
| | | PAIP, DFS70, | interacting protein 1 | |
| | | LEDGF, PSIP2, | GI: 19923652 | |
| | | MGC74712 | NM_033222 | |
| PSMB4 | PSMB4 | PSMB4, HN3, | proteasome (prosome, | WG IVT |
| | | HsN3, PROS26 | macropain) subunit, beta | |
| | | | type, 4 | |
| | | | GI: 22538466 | |
| | | | NM_002796 | |
| PTEN | PTEN | PTEN, BZS, | phosphatase and tensin | WG IVT |
| | | MHAM, TEP1, | homolog GI: 110224474 | |
| | | MMAC1, PTEN1, | NM_000314 | |
| | | MGC11227 | | |
| PTGER3 | PTGER3 | PTGER3, EP3, | prostaglandin E receptor | WG IVT |
| | | EP3e, EP3-I, EP3- | 2 GI: 31881629 | |
| | | II, EP3-IV, EP3-III, | NM_000956 | |
| | | MGC27302, | | |
| | | MGC141828, | | |
| | | MGC141829 | | |
| PTGS1 | PTGS1 | PTGS1, COX1, | prostaglandin- | WG IVT |
| | | COX3, PHS1, | endoperoxide synthase 1 | |
| | | PCOX1, PGHS1, | GI: 18104966 | |
| | | PTGHS, PGG/HS, | NM_000962 | |
| | | PGHS-1 | | |
| QSCN6 | QSCN6 | QSCN6, Q6, | quiescin Q6 | WG IVT |
| | | QSOX1 | GI: 52493187 | |
| | | | NM_002826 | |
| RASSF1 | RASSF1 | RASSF1, 123F2, | Ras association | WG IVT |
| | | RDA32, NORE2A, | (RaIGDS/AF-6) domain | |
| | | RASSF1A, | family 1 | |
| | | REH3P21 | GI: 25777678 | |
| | | | NM_007182 | |
| RCV1 | RCVRN | RCVRN, RCV1 | recoverin GI: 56550117 | WG IVT |
| | | | NM_002903 | |
| RDH11 | RDH11 | RDH11, MDT1, | retinol dehydrogenase | WG IVT |
| | | PSDR1, RALR1, | 11 GI: 20070271 | |
| | | SCALD, ARSDR1, | NM_016026 | |
| | | CGI-82, HCBP12, | | |
| | | FLJ32633 | | |
| RNF14 | RNF14 | RNF14, ARA54, | ring finger protein 14 | WG IVT |
| | | HFB30, FLJ26004, | GI: 34577094 | |
| | | HRIHFB2038 | NM_004290 | |
| RPL23 | RPL23 | RPL23, rpL17, | ribosomal protein L23a | WG IVT |
| | | MGC72008, | GI: 78190460 | |
| | | MGC111167, | NM_000984 | |
| | | MGC117346 | | |
| RPL30 | RPL30 | RPL30 | ribosomal protein L30 | WG IVT |
| | | | GI: 15812218 | |
| | | | NM_000989 | |
| RPS14 | RPS14 | RPS14, EMTB | ribosomal protein S14 | WG IVT |
| | | | (RPS14) | |
| | | | GI: 68160914 | |
| | | | NM_001025070 | |
| RPS6KA1 | RPS6KA1 | RPS6KA1, RSK, | ribosomal protein S6 | WG IVT |
| | | HU-1, RSK1, | kinase, 90 kDa, | |
| | | MAPKAPK1A, S6K- | polypeptide 1 | |
| | | alpha 1 | GI: 56243479 | |
| | | | NM_002953 | |
| SERPINH1 | SERPINH1 | SERPINH1, CBP1, | serpin peptidase | WG IVT |
| | | CBP2, gp46, | inhibitor, clade H (heat | |
| | | AsTP3, HSP47, | shock | |
| | | PIG14, RA-A47, | protein 47), | |
| | | SERPINH2 | member 1, (collagen | |
| | | | binding protein 1) | |
| | | | GI: 32454740 | |
| | | | NM_001235 | |
| SFRP4 | SFRP4 | SFRP4, FRP-4, | secreted frizzled-related | WG IVT |
| | | FRPHE, | protein 4 | |
| | | MGC26498 | GI: 8400733 | |
| | | | NM_003014 | |
| SH3GLB1 | SH3GLB1 | SH3GLB1, Bif-1, | SH3-domain GRB2-like | WG IVT |
| | | CGI-61, KIAA0491, | endophilin B1 | |
| | | dJ612B15.2 | GI: 108936948 | |
| | | | NM_016009 | |
| SPRR1B | SPRR1B | SPRR1B, SPRR1, | small proline-rich protein | WG IVT |
| | | GADD33, | 1B (cornifin) | |
| | | CORNIFIN, | GI: 83582814 | |
| | | MGC61901 | NM_003125 | |
| STEAP | STEAP1 | STEAP1, STEAP, | six transmembrane | WG IVT |
| | | PRSS24, | epithelial antigen of the | |
| | | MGC19484 | prostate 1 | |
| | | | GI: 22027487 | |
| | | | NM_012449 | |
| STIP1 | STIP1 | STIP1, HOP, P60, | stress-induced- | WG IVT |
| | | STI1L, IEF-SSP- | phosphoprotein 1 | |
| | | 3521 | GI: 110225356 | |
| | | | NM_006819 | |
| TMPRSS2 | TMPRSS2 | TMPRSS2, | transmembrane | WG IVT |
| | | PRSS10 | protease, serine 2 | |
| | | | GI: 14602458 | |
| | | | NM_005656 | |
| TOP2A | TOP2A | TOP2A, TOP2, | topoisomerase (DNA) II | WG IVT |
| | | TP2A | alpha 170 kDa | |
| | | | GI: 19913405 | |
| | | | NM_001067 | |
| TP53 | TP53 | TP53, p53, LFS1, | tumor protein p53 (Li- | WG IVT |
| | | TRP53 | Fraumeni syndrome) | |
| | | | GI: 8400737 | |
| | | | NM_000546 | |
| TPD52 | TPD52 | TPD52, D52, N8L, | tumor protein D52 | WG IVT |
| | | PC-1, PrLZ, hD52 | GI: 70608192 | |
| | | | NM_005079 | |
| TRA1(-SP) | HSP90B1 | HSP90B1, ECGP, | heat shock protein | WG IVT |
| | | GP96, TRA1, | 90 kDa beta (Grp94), | |
| | | GRP94 | member 1 GI: 4507676 | |
| | | | NM_003299 | |
| UBE2C | UBE2C | UBE2C, UBCH10, | ubiquitin-conjugating | WG IVT |
| | | dJ447F3.2 | enzyme E2C | |
| | | | GI: 32967292 | |
| | | | NM_007019 | |
| UBQLN1 | UBQLN1 | UBQLN1, DA41, | ubiquilin 1 GI: 44955932 | WG IVT |
| | | DSK2, XDRP1, | NM_013438 | |
| | | PLIC-1, FLJ90054 | | |
| XLKD1 | XLKD1 | XLKD1, HAR, | extracellular link domain | WG IVT |
| | | LYVE-1, CRSBP-1 | containing 1 | |
| | | | GI: 40549450 | |
| | | | NM_006691 NM_016164 | |
| ZWINT | ZWINT | ZWINT, KNTC2AP, | ZW10 interactor | WG IVT |
| | | HZwint-1, | GI: 53729319 | |
| | | MGC117174 | NM_001005413 | |

The Examples provided herein demonstrate that test antigens and test antigens from the 108 test antigens and 8 test antibodies of Table 1, when tested using an immunoassay, detected antibodies or antibody-antigen complexes, respectively, in blood samples of prostate cancer patients. Seventy of the test antigens and all eight of the test antibodies of Table 1 detected autoantibodies or antibody-antigen complexes, respectively, in serum from prostate cancer patients tested using the methods for detecting autoantibodies provided herein.

In addition, ninety-nine target antigens that detected autoantibodies in serum of prostate cancer patients were identified on the PROTOARRAY™ high density protein chip (Invitrogen, Carlsbad, Calif.). The detection of autoantibodies that bind these test antigens (thereby confirmed as autoantigens) in a sample of an individual can be, alone or in combination with the presence or levels of other biomarkers, indicative of cancer. Table 11a provides target antigens identified through the PROTOARRAY™ high density protein chip that demonstrate ability to detect prostate cancer while distinguishing prostate cancer from BPH. Table 11a also indicates whether each antigen has significance for distinguishing High Grade prostate cancer, Low Grade prostate cancer, or both (overall) from BPH. Tables 11b and 11c provide statistical support showing that these antigens are able to distinguish prostate cancer from BPH.

TABLE 11a
Target Antigens Screened on PROTOARRAY ™ Protein Array
having Significance for distinguishing High Grade or Low
Grade prostate cancer from BPH

| | Genbank | UltimateORF ™ | |
|---|---|---|---|
| Gene Symbol | Accession | Clone ID | Significance Call |
| ACAD9 | BC001817 | IOH5174 | Sig in HG |
| B2M | BC032589 | IOH21955 | Sig in HG |
| BMX | NM_001721 | IOH11645 | Sig Overall |
| BRAF | NP_004324 | | Sig in LG |
| BRD3 | BC032124 | IOH23093 | Sig in HG, Sig |
| | | | Overall |
| C10orf65 | BC011916 | IOH12850 | Sig in HG |
| C11orf9 | BC004938 | IOH5465 | Sig in HG |
| C14orf126 | NM_080664 | IOH9768 | Sig in HG |
| C14orf147 | BC021701 | IOH23015 | Sig in HG |
| C1orf36 | NM_183059 | IOH39968 | Sig in HG |
| C4orf15 | NM_024511 | IOH5198 | Sig in HG |
| C7orf31 | BC043269 | IOH25865 | Sig in HG |
| C9orf123 | BC009510 | IOH12049 | Sig in HG, Sig |
| | | | Overall |
| CA14 | NM_012113 | IOH27401 | Sig in HG |
| CASQ2 | BC022288 | IOH12278 | Sig in HG, Sig |
| | | | Overall |
| CD58 | BC005930 | IOH7549 | Sig in HG |
| CDCA8 | BC001651 | IOH3740 | Sig in HG, Sig |
| | | | Overall |
| CDKN1A | BC001935 | IOH5068 | Sig in HG |
| CRABP2 | NM_001878 | IOH1673 | Sig Overall |
| CYB5-M | NM_030579 | IOH5585 | Sig in HG |
| DKFZp434L142 | NM_016613 | IOH11008 | Sig in HG, Sig |
| | | | Overall |
| DTNBP1 | NM_032122 | IOH13153 | Sig in HG |
| DYRK1A | NM_001396 | | Sig in HG |
| EFS | NM_032459 | IOH21413 | Sig in HG |
| FER | NM_005246 | | Sig in HG |
| FHIT | NM_002012 | IOH21676 | Sig Overall |
| FKBP6 | BC036817 | IOH22107 | Sig in HG |
| FLJ10052 | BC004888 | IOH5668 | Sig in HG |
| FLJ13150 | BC039014 | IOH26125 | Sig in HG |
| FLJ13910 | NM_022780 | IOH13276 | Sig in HG |
| FLJ30294 | BC020898 | IOH13022 | Sig in HG |
| FLJ30473 | BC032485 | IOH21724 | Sig in HG |
| FLJ32884 | BC033790 | IOH21793 | Sig in LG |
| FLJ44216 | BC032390 | IOH27534 | Sig Overall |
| FRMD3 | BC023560 | IOH27849 | Sig in LG |
| FTL | BC016715 | IOH27895 | Sig Overall |
| HADHSC | BC000306 | IOH3456 | Sig Overall |
| HCK | BC014435 | IOH14630 | Sig in HG |
| HEY1 | BC001873 | IOH4800 | Sig in HG, Sig |
| | | | Overall |
| HLA-DRB2 | BC033827 | IOH21889 | Sig in HG |
| HNRPK | NM_002140 | IOH3670 | Sig Overall |
| HSPA4 | BC002526 | IOH4058 | Sig in HG |
| IL17RB | BC000980 | IOH2952 | Sig in HG, Sig |
| | | | Overall |
| JDP2 | NM_130469 | IOH28073 | Sig in HG, Sig |
| | | | Overall |
| LARP | BC033856 | IOH21797 | Sig in HG, Sig |
| | | | Overall |
| LEPREL1 | BC005029 | IOH6657 | Sig Overall |
| LIG3 | NM_013975 | IOH40893 | Sig in HG |
| LOC196394 | NM_207337 | IOH40127 | Sig in HG, Sig |
| | | | Overall |
| LOC441046 | BC025996 | IOH10875 | Sig in HG |
| MGC31967 | NM_174923 | IOH14835 | Sig in HG |
| MGC40168 | NM_153709 | IOH21517 | Sig in HG, Sig |
| | | | Overall |
| MGC52010 | NM_194326 | IOH26706 | Sig in HG |
| MGC59937 | NM_199001 | IOH28105 | Sig in HG |
| MLKL | BC028141 | IOH21529 | Sig in HG |
| MPG | BC014991 | IOH12177 | Sig in HG |
| MPPE1 | BC002877 | IOH5717 | Sig in HG |
| MS4A4A | NM_024021 | IOH36738 | Sig in HG |
| MTHFD2 | BC017054 | IOH10366 | Sig in HG |
| MVD | NM_002461 | IOH4651 | Sig in HG |
| MYC | BC000141 | IOH2954 | Sig in HG |
| MYLC2PL | BC002778 | IOH5313 | Sig Overall |
| NAP1L2 | BC026325 | IOH11158 | Sig Overall |
| PDE4DIP | BC025406 | IOH11226 | Sig in HG |
| PDYN | NM_024411 | IOH11247 | Sig in HG |
| PPAP2B | BC009196 | IOH12943 | Sig in HG |
| PPIA | BC007104 | IOH7532 | Sig in HG |
| PRKACB | BC035058 | IOH27691 | Sig Overall |
| PSMD11 | NM_002815 | IOH3459 | Sig in LG |
| PTGS2 | NM_000963 | IOH11237 | Sig in LG |
| RFX5 | NM_000449 | IOH10040 | Sig in HG |
| RNF5 | NM_006913 | IOH3743 | Sig in HG, Sig |
| | | | Overall |
| RPL14 | BC005134 | IOH5666 | Sig Overall |
| RPS19 | NM_001022 | IOH4572 | Sig Overall |
| RPS6KA3 | NM_004586 | | Sig in HG |
| RPS6KC1 | NM_012424 | | Sig Overall |
| RRAGB | BC034726 | IOH25776 | Sig in HG |
| SERPINI2 | NM_006217 | IOH11838 | Sig in HG |
| SFRS7 | BC000997 | IOH2939 | Sig in HG |
| SMARCD2 | BC018953 | IOH13650 | Sig in HG |
| SMN2 | NM_017411 | IOH10903 | Sig in LG |
| SMR3B | NM_006685 | IOH11229 | Sig in HG |
| SNAI2 | NM_003068 | IOH10082 | Sig in HG |
| SPG21 | NM_016630 | IOH4511 | Sig Overall |
| SPRR1B | BC056240 | IOH29466 | Sig Overall |
| SRP9 | BC021995 | IOH14627 | Sig in HG |
| TTYH2 | BC004233 | IOH5231 | Sig in HG, Sig |
| | | | Overall |
| TXNL4A | NM_006701 | IOH3749 | Sig Overall |
| TYRO3 | NM_006293 | | Sig in HG |
| UBOX5 | NM_199415 | IOH26936 | Sig in HG |
| UCK2 | NM_012474 | IOH40599 | Sig in LG |
| URG4 | BC018426 | IOH9673 | Sig in HG |
| UROS | NM_000375 | IOH4136 | Sig Overall |
| UXS1 | BC009819 | IOH12608 | Sig Overall |
| VAPB | NM_004738 | IOH4934 | Sig in HG |
| VIPR2 | NM_003382 | IOH9624 | Sig in HG |
| WDR4 | NM_033661 | IOH6391 | Sig in HG, Sig |
| | | | Overall |
| ZCCHC4 | BC016914 | IOH10995 | Sig in HG |
| ZNF581 | NM_016535 | IOH4783 | Sig in HG, Sig |
| | | | Overall |

TABLE 11b
Target Antigens Screened on PROTOARRAY ™ Protein Array having
Significance for distinguishing High Grade or Low Grade prostate cancer from BPH;
Statistics including P-values

| | Low Grade | High Grade | | | |
|---|---|---|---|---|---|
| | Cancer/Normal | Cancer/Normal | All PCA vs BPH | HG PCA vs | LG PCA vs |
| Gene Symbol | Ratio | Ratio | P-Value | BPH P-Value | BPH P-Value |
| ACAD9 | 1.162470024 | 0.757246377 | 0.05988135 | 0.000969619 | 0.259609431 |
| B2M | 1.219371915 | 0.956939082 | 0.023031288 | 0.000161603 | 0.250773994 |
| BMX | 1.23719588 | 0.799912988 | 0.000707711 | 0.003393665 | 0.054227197 |
| BRAF | 1.06137257 | 0.756860044 | 0.03294643 | 0.127989657 | 0.000357228 |
| BRD3 | 1.007407407 | 1.46509851 | 0.00022316 | 0.000161603 | 0.003611971 |
| C10orf65 | 1.27312196 | 0.778762307 | 0.002055424 | 0.000969619 | 0.057791538 |
| C11orf9 | 1.13630708 | 1.341577122 | 0.001913559 | 0.000969619 | 0.015440184 |
| C14orf126 | 1.634055501 | 0.804034796 | 0.042772393 | 0.000969619 | 0.187306502 |
| C14orf147 | 1.353750582 | 0.908549821 | 0.004574786 | 0.000969619 | 0.03989045 |
| C1orf36 | 0.970859211 | 0.772121966 | 0.002055424 | 0.000969619 | 0.018059856 |
| C4orf15 | 1.243298599 | 0.706382334 | 0.004574786 | 0.000969619 | 0.057791538 |
| C7orf31 | 1.276783872 | 0.971358584 | 0.001301768 | 0.000161603 | 0.049122807 |
| C9orf123 | 1.333791089 | 0.783155116 | 0.000317583 | 0.000969619 | 0.004437564 |
| CA14 | 1.197577425 | 0.759441992 | 0.058881043 | 0.000969619 | 0.204929745 |
| CASQ2 | 1.300961325 | 0.853836373 | 0.000223218 | 0.000969619 | 0.015440184 |
| CD58 | 2.010870829 | 0.946903077 | 0.005988135 | 0.000161603 | 0.105572755 |
| CDCA8 | 1.48758564 | 0.735950982 | 0.00022316 | 0.000161603 | 0.014447884 |
| CDKN1A | 1.312358322 | 0.790184407 | 0.002863891 | 0.000969619 | 0.105572755 |
| CRABP2 | 1.339278029 | 0.907247212 | 0.0003608 | 0.009049774 | 0.003929507 |
| CYB5-M | 0.993521615 | 0.477181166 | 0.01197627 | 0.000161603 | 0.187306502 |
| DKFZp434L142 | 1.208883709 | 0.907434598 | 0.000317583 | 0.000969619 | 0.004437564 |
| DTNBP1 | 1.518970007 | 1.030859029 | 0.009484313 | 0.000161603 | 0.259609431 |
| DYRK1A | 1.372657432 | 1.010271961 | 0.042772393 | 0.000161603 | 0.306501548 |
| EFS | 2.247215006 | 0.48469795 | 0.005988135 | 0.000969619 | 0.014447884 |
| FER | 1.324652778 | 0.955782313 | 0.01197627 | 0.000969619 | 0.156918314 |
| FHIT | 1.039032959 | 0.743245033 | 0.000707711 | 0.003393665 | 0.003215051 |
| FKBP6 | 1.312099253 | 0.857584561 | 0.002055424 | 0.000969619 | 0.049122807 |
| FLJ10052 | 2.431110974 | 0.702468544 | 0.009484313 | 0.000969619 | 0.03989045 |
| FLJ13150 | 1.215104062 | 0.770332481 | 0.003932941 | 0.000969619 | 0.049122807 |
| FLJ13910 | 1.424118335 | 0.983937883 | 0.001913559 | 0.000969619 | 0.156918314 |
| FLJ30294 | 1.357971014 | 0.973864326 | 0.009484313 | 0.000969619 | 0.057791538 |
| FLJ30473 | 2.007543999 | 0.540415322 | 0.034106334 | 0.000969619 | 0.296181631 |
| FLJ32884 | 1.143609646 | 0.626325474 | 0.019690961 | 0.278280543 | 0.000357228 |
| FLJ44216 | 1.248492407 | 0.90009307 | 0.000707711 | 0.003393665 | 0.003215051 |
| FRMD3 | 1.146829664 | | 0.001301768 | 0.014705882 | 0.000722394 |
| FTL | 0.88004925 | 0.761439346 | 0.000223218 | 0.003393665 | 0.003215051 |
| HADHSC | 1.551796828 | 0.880630631 | 0.000235304 | 0.00210084 | 0.010216718 |
| HCK | 1.330952381 | 0.696443342 | 0.003932941 | 0.000161603 | 0.023839009 |
| HEY1 | 1.397489485 | 1.102228373 | 0.0003608 | 0.000161603 | 0.024886878 |
| HLA-DRB2 | 1.215223401 | 0.629726665 | 0.002863891 | 0.000161603 | 0.003929507 |
| HNRPK | 1.303875824 | 0.773978726 | 0.000557901 | 0.052521008 | 0.014447884 |
| HSPA4 | 1.580284949 | 0.992612842 | 0.001913559 | 0.000969619 | 0.015440184 |
| IL17RB | 1.282353347 | 0.93388687 | 0.00022316 | 0.000161603 | 0.009883306 |
| JDP2 | 1.240702174 | 0.934734513 | 0.000849575 | 0.000969619 | 0.057791538 |
| LARP | 1.020773383 | 1.600998404 | 0.000557901 | 0.000969619 | 0.014447884 |
| LEPREL1 | 0.997990926 | 0.728299735 | 0.000707711 | 0.003393665 | 0.001309836 |
| LIG3 | 0.810788225 | 0.790472468 | 0.018485112 | 0.000969619 | 0.049122807 |
| LOC196394 | 1.150393767 | 0.717851917 | 0.000849575 | 0.000969619 | 0.018059856 |
| LOC441046 | 0.871319142 | 0.697274808 | 0.001019651 | 0.000969619 | 0.023839009 |
| MGC31967 | 1.702403255 | 0.739953427 | 0.023031288 | 0.000161603 | 0.147368421 |
| MGC40168 | 0.749691945 | 0.882877794 | 0.000223218 | 0.000969619 | 0.003215051 |
| MGC52010 | 1.089363462 | 0.766755587 | 0.001301768 | 0.000969619 | 0.049122807 |
| MGC59937 | 1.433770866 | 1.171278591 | 0.076990307 | 0.000161603 | 0.296181631 |
| MLKL | 1.545161861 | 0.623150295 | 0.002863891 | 0.000161603 | 0.147368421 |
| MPG | 1.621703212 | 0.93860869 | 0.005988135 | 0.000161603 | 0.014447884 |
| MPPE1 | 1.137113311 | 0.679554968 | 0.001301768 | 0.000161603 | 0.018059856 |
| MS4A4A | 1.250713572 | 1.1002849 | 0.001913559 | 0.000161603 | 0.015440184 |
| MTHFD2 | 1.573891626 | 0.959472228 | 0.002863891 | 0.000161603 | 0.102167183 |
| MVD | 0.982767902 | 0.761473708 | 0.004574786 | 0.000969619 | 0.003215051 |
| MYC | 1.294859551 | 1.060884219 | 0.005988135 | 0.000161603 | 0.105572755 |
| MYLC2PL | 1.28086945 | 0.770674519 | 0.0003608 | 0.020361991 | 0.023839009 |
| NAP1L2 | 1.135174419 | 0.76985755 | 0.000334293 | 0.009049774 | 0.03989045 |
| PDE4DIP | 1.222619048 | 0.832978193 | 0.058881043 | 0.000969619 | 0.137254902 |
| PDYN | 1.343532744 | 0.749279144 | 0.013765295 | 0.000969619 | 0.102167183 |
| PPAP2B | 1.487720911 | 0.725823454 | 0.001913559 | 0.000161603 | 0.147368421 |
| PPIA | 0.894068691 | | 0.003932941 | 0.000969619 | 0.051083591 |
| PRKACB | 1.24862016 | 0.840965587 | 0.000223218 | 0.003393665 | 0.015440184 |
| PSMD11 | 0.903461389 | 0.733500484 | 0.05988135 | 0.207983193 | 0.000770025 |
| PTGS2 | 1.165972222 | 0.706594445 | 0.002863891 | 0.073529412 | 0.000722394 |
| RFX5 | 2.30012442 | 0.609409763 | 0.005725735 | 0.000969619 | 0.052115583 |
| RNF5 | 0.918109106 | 0.739568234 | 0.000849575 | 0.000969619 | 0.004437564 |
| RPL14 | 1.048172957 | 0.648122651 | 0.0003608 | 0.009857789 | 0.024886878 |
| RPS19 | 2.27209198 | 0.613443063 | 0.000849575 | 0.009049774 | 0.018059856 |
| RPS6KA3 | 1.295793226 | 0.899175064 | 0.01197627 | 0.000969619 | 0.147368421 |
| RPS6KC1 | 1.063906337 | 0.772026543 | 0.000235304 | 0.020361991 | 0.010216718 |
| RRAGB | 1.514367816 | 0.705171652 | 0.005988135 | 0.000161603 | 0.049122807 |
| SERPINI2 | 1.332480407 | 1.035150924 | 0.002863891 | 0.000161603 | 0.156918314 |
| SFRS7 | 2.065030947 | 1.51100029 | 0.113756025 | 0.000969619 | 0.187306502 |
| SMARCD2 | 1.246801706 | 0.936639229 | 0.001913559 | 0.000161603 | 0.052115583 |
| SMN2 | 1.059886664 | 0.612138501 | 0.003291672 | 0.009049774 | 0.000770025 |
| SMR3B | 1.298064611 | 1.065819421 | 0.023031288 | 0.000161603 | 0.187306502 |
| SNAI2 | 1.306347607 | 0.959687052 | 0.002055424 | 0.000161603 | 0.057791538 |
| SPG21 | 1.432676317 | 1.012578455 | 0.000317583 | 0.052521008 | 0.015440184 |
| SPRR1B | 0.853978677 | 1.527526395 | 0.000707711 | 0.074660633 | 0.003215051 |
| SRP9 | 1.577757079 | 0.990385933 | 0.01197627 | 0.000161603 | 0.102167183 |
| TTYH2 | 1.392587894 | 1.165611587 | 0.00022316 | 0.000161603 | 0.03989045 |
| TXNL4A | 0.744509392 | 1.281679936 | 0.000849575 | 0.009857789 | 0.004437564 |
| TYRO3 | 4.096516716 | | 0.023031288 | 0.000969619 | 0.049122807 |
| UBOX5 | 1.472284007 | 0.935727116 | 0.009484313 | 0.000969619 | 0.153147575 |
| UCK2 | 0.751419118 | 0.882890123 | 0.004574786 | 0.009857789 | 0.000770025 |
| URG4 | 1.232855865 | 1.917607328 | 0.002863891 | 0.000969619 | 0.014447884 |
| UROS | 1.462585682 | 0.873990628 | 0.000849575 | 0.009049774 | 0.004437564 |
| UXS1 | 1.209117235 | 1.030439466 | 0.000707711 | 0.003393665 | 0.137254902 |
| VAPB | 1.4075032 | 0.905413431 | 0.009484313 | 0.000161603 | 0.057791538 |
| VIPR2 | 1.624015347 | 0.782106501 | 0.043280283 | 0.000969619 | 0.147368421 |
| WDR4 | 1.284354133 | 0.671563203 | 0.000557901 | 0.000969619 | 0.014447884 |
| ZCCHC4 | 1.492037651 | 0.840563712 | 0.002863891 | 0.000161603 | 0.049122807 |
| ZNF581 | 1.29159949 | 0.856237167 | 0.000849575 | 0.000969619 | 0.003611971 |

TABLE 11c
Target Antigens Screened on PROTOARRAY ™ Protein Array having
Significance for distinguishing High Grade or Low Grade prostate
cancer from BPH; Statistics including prevalence

| | | All PCa | HG PCA | LG PCA |
|---|---|---|---|---|
| Gene Symbol | BPH Prevalence | Prevalence | Prevalence | Prevalence |
| ACAD9 | 21.43% | 40.91% | 85.71% | 40.00% |
| B2M | 14.29% | 36.36% | 85.71% | 50.00% |
| BMX | 28.57% | 77.27% | 85.71% | 80.00% |
| BRAF | 71.43% | 50.00% | 28.57% | 30.00% |
| BRD3 | 21.43% | 63.64% | 85.71% | 60.00% |
| C10orf65 | 21.43% | 63.64% | 85.71% | 50.00% |
| C11orf9 | 28.57% | 72.73% | 85.71% | 70.00% |
| C14orf126 | 14.29% | 31.82% | 85.71% | 80.00% |
| C14orf147 | 21.43% | 59.09% | 85.71% | 70.00% |
| C1orf36 | 85.71% | 40.91% | 28.57% | 50.00% |
| C4orf15 | 21.43% | 59.09% | 85.71% | 50.00% |
| C7orf31 | 14.29% | 54.55% | 85.71% | 40.00% |
| C9orf123 | 21.43% | 72.73% | 85.71% | 70.00% |
| CA14 | 35.71% | 59.09% | 85.71% | 60.00% |
| CASQ2 | 28.57% | 81.82% | 85.71% | 70.00% |
| CD58 | 14.29% | 45.46% | 85.71% | 80.00% |
| CDCA8 | 14.29% | 63.64% | 85.71% | 50.00% |
| CDKN1A | 14.29% | 50.00% | 85.71% | 80.00% |
| CRABP2 | 64.29% | 13.64% | 28.57% | 20.00% |
| CYB5-M | 14.29% | 40.91% | 85.71% | 80.00% |
| DKFZp434L142 | 21.43% | 72.73% | 85.71% | 70.00% |
| DTNBP1 | 21.43% | 54.55% | 85.71% | 40.00% |
| DYRK1A | 14.29% | 31.82% | 85.71% | 80.00% |
| EFS | 14.29% | 45.46% | 85.71% | 50.00% |
| FER | 14.29% | 40.91% | 85.71% | 70.00% |
| FHIT | 78.57% | 36.36% | 28.57% | 40.00% |
| FKBP6 | 85.71% | 40.91% | 28.57% | 70.00% |
| FLJ10052 | 21.43% | 54.55% | 85.71% | 70.00% |
| FLJ13150 | 42.86% | 9.09% | 28.57% | 70.00% |
| FLJ13910 | 28.57% | 72.73% | 85.71% | 70.00% |
| FLJ30294 | 21.43% | 54.55% | 85.71% | 50.00% |
| FLJ30473 | 85.71% | 59.09% | 28.57% | 70.00% |
| FLJ32884 | 28.57% | 59.09% | 71.43% | 90.00% |
| FLJ44216 | 28.57% | 77.27% | 85.71% | 80.00% |
| FRMD3 | 14.29% | 54.55% | 57.14% | 70.00% |
| FTL | 78.57% | 22.73% | 28.57% | 30.00% |
| HADHSC | 50.00% | 95.46% | 71.43% | 90.00% |
| HCK | 42.86% | 9.09% | 28.57% | 20.00% |
| HEY1 | 42.86% | 90.91% | 85.71% | 80.00% |
| HLA-DRB2 | 92.86% | 54.55% | 28.57% | 20.00% |
| HNRPK | 92.86% | 45.46% | 57.14% | 60.00% |
| HSPA4 | 28.57% | 72.73% | 85.71% | 70.00% |
| IL17RB | 14.29% | 63.64% | 85.71% | 80.00% |
| JDP2 | 21.43% | 68.18% | 85.71% | 50.00% |
| LARP | 14.29% | 59.09% | 85.71% | 50.00% |
| LEPREL1 | 78.57% | 27.27% | 28.57% | 20.00% |
| LIG3 | 85.71% | 54.55% | 28.57% | 70.00% |
| LOC196394 | 85.71% | 36.36% | 28.57% | 50.00% |
| LOC441046 | 50.00% | 9.09% | 28.57% | 20.00% |
| MGC31967 | 14.29% | 36.36% | 85.71% | 80.00% |
| MGC40168 | 78.57% | 22.73% | 28.57% | 30.00% |
| MGC52010 | 14.29% | 54.55% | 85.71% | 40.00% |
| MGC59937 | 14.29% | 27.27% | 85.71% | 70.00% |
| MLKL | 92.86% | 54.55% | 28.57% | 80.00% |
| MPG | 92.86% | 59.09% | 28.57% | 60.00% |
| MPPE1 | 14.29% | 54.55% | 85.71% | 60.00% |
| MS4A4A | 78.57% | 31.82% | 28.57% | 40.00% |
| MTHFD2 | 14.29% | 50.00% | 85.71% | 90.00% |
| MVD | 85.71% | 45.46% | 28.57% | 30.00% |
| MYC | 14.29% | 45.46% | 85.71% | 80.00% |
| MYLC2PL | 64.29% | 13.64% | 28.57% | 20.00% |
| NAP1L2 | 71.43% | 22.73% | 28.57% | 40.00% |
| PDE4DIP | 71.43% | 45.46% | 28.57% | 50.00% |
| PDYN | 71.43% | 95.46% | 85.71% | 90.00% |
| PPAP2B | 28.57% | 72.73% | 85.71% | 80.00% |
| PPIA | 64.29% | 95.46% | 85.71% | 90.00% |
| PRKACB | 78.57% | 22.73% | 28.57% | 40.00% |
| PSMD11 | 85.71% | 63.64% | 28.57% | 30.00% |
| PTGS2 | 14.29% | 50.00% | 42.86% | 70.00% |
| RFX5 | 57.14% | 86.36% | 85.71% | 60.00% |
| RNF5 | 85.71% | 36.36% | 28.57% | 40.00% |
| RPL14 | 42.86% | 90.91% | 71.43% | 80.00% |
| RPS19 | 21.43% | 68.18% | 85.71% | 60.00% |
| RPS6KA3 | 14.29% | 40.91% | 85.71% | 80.00% |
| RPS6KC1 | 57.14% | 9.09% | 28.57% | 20.00% |
| RRAGB | 14.29% | 45.46% | 85.71% | 40.00% |
| SERPINI2 | 14.29% | 50.00% | 85.71% | 70.00% |
| SFRS7 | 50.00% | 68.18% | 85.71% | 30.00% |
| SMARCD2 | 28.57% | 72.73% | 85.71% | 60.00% |
| SMN2 | 71.43% | 27.27% | 28.57% | 30.00% |
| SMR3B | 14.29% | 36.36% | 85.71% | 80.00% |
| SNAI2 | 21.43% | 63.64% | 85.71% | 50.00% |
| SPG21 | 21.43% | 72.73% | 57.14% | 70.00% |
| SPRR1B | 78.57% | 27.27% | 28.57% | 30.00% |
| SRP9 | 14.29% | 40.91% | 85.71% | 90.00% |
| TTYH2 | 14.29% | 63.64% | 85.71% | 70.00% |
| TXNL4A | 85.71% | 36.36% | 42.86% | 40.00% |
| TYRO3 | 92.86% | 68.18% | 28.57% | 40.00% |
| UBOX5 | 85.71% | 50.00% | 28.57% | 70.00% |
| UCK2 | 85.71% | 45.46% | 57.14% | 30.00% |
| URG4 | 14.29% | 50.00% | 85.71% | 50.00% |
| UROS | 85.71% | 36.36% | 28.57% | 40.00% |
| UXS1 | 28.57% | 77.27% | 85.71% | 50.00% |
| VAPB | 21.43% | 54.55% | 85.71% | 50.00% |
| VIPR2 | 57.14% | 81.82% | 85.71% | 80.00% |
| WDR4 | 14.29% | 59.09% | 85.71% | 50.00% |
| ZCCHC4 | 14.29% | 50.00% | 85.71% | 40.00% |
| ZNF581 | 85.71% | 36.36% | 28.57% | 60.00% |

The invention therefore provides test antigens and target antigens that specifically bind autoantibodies present in samples of individuals. The invention also provides test antibodies and target antibodies that specifically bind autoantigens present in samples from individuals, in which the target antibodies can be used to detect autoantibodies bound to the recognized autoantigens. The invention provides methods of using target antigens and target antibodies, collectively referred to herein as autoantibody capture molecules, to detect autoantibodies in samples from individuals, and methods of using autoantibody capture molecules to detect cancer by detecting autoantibodies in samples from individuals. The invention also provides methods of using autoantibody capture molecules to detect prostate cancer by detecting autoantibodies, and biomarker detection panels that comprise autoantibody capture molecules for detecting prostate cancer autoantibody biomarkers. The invention provides methods of detecting, diagnosing, prognosing, staging, and monitoring prostate cancer using the identified target antigens and target antibodies, provides biomarker detection panels for detecting autoantibodies, and also provides kits that include autoantibody capture molecules.

A target antibody used in any of the methods and compositions provided herein can be any antibody that specifically binds an autoantigen in a sample from an individual, in which the autoantigen can be bound by the capture antibody while bound to an autoantibody from the sample. Target antibodies include, for example, the antibodies listed in Table 1, such as antibodies to ACPP, BCL2, CXCR4, IL-6, IL-8, PSA(F) (free PSA), PSA(T) (total, or free plus bound PSA), or PTGER2, that are able to detect autoantibodies by binding antigen-antibody complexes, and also include antibodies to any of the proteins of Table 1 or Table 11a, in which the antibodies are able to detect autoantibodies in a sample by specifically binding to an autoantigen-autoantibody complex present in a sample.

A target antigen in any of the aspects or embodiments of the invention can be an entire protein, such as the protein referred to as a target antigen, including a precursor of the protein, or an unprocessed form, processed form, or post-translationally modified form of the protein, or a form of the protein that is not post-translationally modified, or a form of the protein that is partially, atypically, or abnormally post-translationally modified. A target antigen used in the methods or compositions provided herein can be an isoform of the designated protein (e.g., a splice variant), or an allelic variant, or a target antigen can be an epitope-containing fragment of the protein named as a target antigen.

The Genbank sequence identifiers and accession numbers provided in the tables that list target antigens do not limit the proteins to being encoded by those specific sequences. In particular, the identified proteins may, at a future date, have updated sequences submitted, or may have isoforms, allelic variants, homologs, etc., that are also included as a target protein (target antigen) identified by the particular Genbank sequence identifiers or accession numbers. Sequence variants of antigens or antigen fragments of proteins in the referenced tables also include peptides and polypeptides having at least 70%, at least 75%, at least 80%, at least 85%, at least 90%, at least 95%, at least 96%, at least 97%, at least 98%, or at least 99% amino acid sequence homology to a protein or a fragment thereof that is at least four amino acids in length, in which the sequence variant binds an antibody that recognizes the target antigen listed in the table.

Specifically included in the term “target antigen” is a molecule that comprises an epitope-containing fragment of the protein named as a target antigen (or a sequence variant thereof), such that the molecule is specifically recognized by an antibody that recognizes the target antigen epitope. A molecule that comprises an epitope-containing fragment can be any type of molecule, and can be a polymer, including a synthetic polymer, or a biomolecule such as a polypeptide, nucleic acid, peptide nucleic acid, etc.

A fragment of a protein that includes an epitope recognized by an antibody can be at least 3, 4, 5, 6, 7, 8, 9, 10, 11, 12, 13, 14, 15, 20, 25, 30, 35, 40, 45, 50, 75, 100, 150, 200, 250, 300, 350, 400, 450, 500, 750, or 1000 amino acids in length, where the amino acids in the fragment correspond to consecutive amino acids in the full length protein sequence. Preferably, the fragment is at least 15 amino acids in length. A fragment that includes an epitope recognized by an antibody can be greater than 1000 amino acids in length. The fragment can also be between 5,10, 15, 20, 25, 50, 75, 100, 150, 200, 250, 300, 350, 400, 450, 500, 600, 700, 800, 900, 1000, or 2000 amino acids and one amino acid less than the entire length of an autoantigen. Typically, such epitopes are characterized in advance such that it is known that autoantibodies for a given antigen recognize the epitope. Methods for epitope mapping are well known in the art.

An autoantibody capture molecule that is a target antibody is an antibody that can detect an autoantibody in a sample. An autoantibody capture molecule that can detect an autoantibody in a sample detects an autoantibody that is complexed to an autoantigen to which the target antibody specifically binds. Antibodies can be tested for their ability to bind autoantigen-autoantibody complexes by methods disclosed herein, for example, by detecting binding using a directly or indirectly labeled species-specific secondary antibody that recognizes antibodies of the species from which the sample has been provided.

An autoantibody capture molecule can be, for example, any of the target antigens or target antibodies listed in Table 1 or Table 11a, or can be an antibody to any of the target antigens of Table 1 or Table 11a. For example, Table 2 lists a set of 55 proteins selected from Table 1 which are believed to be particularly useful antigens in detecting prostate cancer. Markers from Table 1 that were detectable in prostate cancer serum and exhibited signals that were at least two times background are provided in Table 3. Table 4 lists a set of 83 proteins selected from Table 1, including proteins from Table 2, which are believed to be particularly useful antigens in detecting prostate cancer. In some embodiments an autoantibody capture molecule is a target antigen of Table 2, or is an antibody to any of the target antigens provided in Table 2, in which the antibody can specifically bind an autoantibody-autoantigen complex that includes a target antigen of Table 2. In some embodiments and antibody capture molecule is a is of Table 3. In some embodiments an autoantibody capture molecule is a target antigen of Table 4, or is an antibody to any of the target antigens provided in Table 4, in which the antibody can specifically bind an autoantibody-autoantigen complex that includes a target antigen of Table 4.

TABLE 2
Proteins of Interest
Current Term and
Hugo Symbol Term

AGR2
ALOX15
AZGP1
BCLG
BDKRB2
BIRC5
BRD2
CCKBR
CCNA1
CCNB1
CCND1
CD151
CLDN3
CLDN4
COVA1
CUL4A
EIF3S3
EIF4G1
ELAC1
ETS2
FLT1
HEYL
HIP2
HOXB13
HPN
MAD1L1
MICB
MLH1
NCAM2
NRP1
NUCB1
PDLIM1
PIM1
PRSS8
PSAP
PSCA
PSMB4
PTGER3
PTGS1
QSCN6
RASSF1
RDH11
RNF14
RPL23
RPL30
RPS14
RPS6KA1
SFRP4
SH3GLB1
SPRR1B
STEAP
TOP2A
UBE2C
UBQLN1
ZWINT

TABLE 3
Autoantibody Capture Molecules that Detected Autoantibodies in Sera of
PCa Patients

| MARKER | Genbank ID | |
|---|---|---|
| ACPP | NM_001099 | antigen |
| AMACR | NM_014707 | antigen |
| AZGP1 | NM_001185 | antigen |
| BCL2 | NM_000633 | antigen |
| BCLG | NM_030766 | antigen |
| BDKRB2 | NM_000623 | antigen |
| BIRC5 | NM_001012270 | antigen |
| CAV3 | NM_001753 | antigen |
| CCNA1 | NM_003914 | antigen |
| CCNB1 | NM_031966 | antigen |
| CCND1 | NM_053056 | antigen |
| CD151 | NM_004357 | antigen |
| CD164 | NM_006016 | antigen |
| CLDN3 | NM_001306 | antigen |
| COVA1 | NM_006375 | antigen |
| EGFR | NM_005228 | antigen |
| EIF3S3 | NM_003756 | antigen |
| EIF4G1 | NM_182917 | antigen |
| ENO1 | NM_001428 | antigen |
| ERBB2 | NM_001005862 | antigen |
| ERG | NM_004449 | antigen |
| ETS2 | NM_005239 | antigen |
| HEYL | NM_014571 | antigen |
| HIP1 | NM_005338 | antigen |
| HMGA2 | NM_003484 | antigen |
| HSPA1A | NM_005345 | antigen |
| HSPA5 | NM_005347 | antigen |
| HSPB1 | NM_001540 | antigen |
| HSPD1 | NM_002156 | antigen |
| IMP-2 | NM_006548 | antigen |
| IMP-3 | NM_006559 | antigen |
| KDR | NM_002253 | antigen |
| KHDRBS1 | NM_006559 | antigen |
| MAD1L1 | NM_002358 | antigen |
| MET | NM_000245 | antigen |
| MICB | NM_005931 | antigen |
| MLH1 | NM_000249 | antigen |
| MMP9 | NM_004994 | antigen |
| MUC1 | NM_002456 | antigen |
| MYC | NM_002467 | antigen |
| NCAM2 | NM_004540 | antigen |
| NRP1 | NM_015022 | antigen |
| NUCB1 | NM_006184 | antigen |
| PCNA | NM_182649 | antigen |
| PRL | NM_000948 | antigen |
| PRSS8 | NM_002773 | antigen |
| PSA | NM_145864 | antigen |
| PSAP | NM_002778 | antigen |
| PSIP1 | NM_033222 | antigen |
| PSMB4 | NM_002796 | antigen |
| PTEN | NM_000314 | antigen |
| PTGER3 | NM_000956 | antigen |
| QSCN6 | NM_002826 | antigen |
| RASSF1 | NM_007182 | antigen |
| RCV1 | NM_002903 | antigen |
| RDH11 | NM_016026 | antigen |
| RNF14 | NM_004290 | antigen |
| RPL30 | NM_000989 | antigen |
| RPS6KA1 | NM_002953 | antigen |
| SH3GLB1 | NM_016009 | antigen |
| SPRR1B | NM_003125 | antigen |
| STEAP | NM_012449 | antigen |
| STIP1 | NM_006819 | antigen |
| TMPRSS2 | NM_005656 | antigen |
| TOP2A | NM_001067 | antigen |
| TP53 | NM_000546 | antigen |
| TPD52 | NM_005079 | antigen |
| TRA1(-SP) | NM_003299 | antigen |
| XLKD1 | NM_016164 | antigen |
| ZWINT | NM_001005413 | antigen |
| a-ACPP | | antibody |
| a-BCL2 | | antibody |
| a-CXCR4 | | antibody |
| a-II-6-1 | | antibody |
| a-II-8-1 | | antibody |
| a-PSA (F) | | antibody |
| a-PSA(T) | | antibody |
| a-Pter2 | | antibody |

TABLE 4
Proteins of Interest
Current Term

ACPP
AGR2
AKT1
ALOX15
AZGP1
BCL2
BCLG
BDKRB2
BIRC5
BRD2
CAV3
CCKBR
CCNA1
CCNB1
CCND1
CD151
CD164
CDKN2A
CLDN3
CLDN4
COVA1
CUL4A
CXCR4
EDNRB
EGFR
EIF3S3
EIF4G1
ELAC1
EP-CAM
ERBB2
ETS2
FASN
FLT1
GDF15
HEYL
HIP2
HMGA2
HOXB13
HPN
HSPA1A
HSPB1
HSPD1
IMP-3
KDR
LGALS8
MAD1L1
MDM2
MICB
MLH1
NCAM2
NRP1
NUCB1
PCNA
PDLIM1
PECAM1
PIM1
PRSS8
PSAP
PSCA
PSMB4
PTEN
PTGER3
PTGS1
QSCN6
RASSF1
RDH11
RNF14
RPL23
RPL30
RPS14
RPS6KA1
SERPINH1
SFRP4
SH3GLB1
SPRR1B
STEAP
STIP1
TOP2A
TRA1
UBE2C
UBQLN1
XLKD1
ZWINT

In certain embodiments, one or more diagnostic (or prognostic) biomarkers, such as one or more autoantibody biomarkers, are correlated to a condition or disease by the presence or absence of the biomarker(s). In other embodiments, threshold level(s) of a diagnostic or prognostic biomarker(s) can be established, and the level of the biomarker(s) in a sample can be compared to the threshold level(s). Levels can be relative or absolute, and are preferably normalized with respect to one or more controls.

In the methods provided herein, the test sample can be contacted with an autoantibody capture molecule provided in solution phase, or the autoantibody capture molecule can be provided bound to a solid support. The sample can be diluted or concentrated, or subjected to one or more processing steps prior to contacting with an autoantibody capture molecule. In some preferred embodiments, the sample is a serum sample that is diluted in a binding buffer. The dilution can be any useful dilution for obtaining a detectable binding signal with acceptable background, such as, for example, from no dilution to 1:10,000, 1:1 to 1:1,000, or from 1:2 to 1:500, or from 1:5 to 1:200, or from 1:10 to 1:100. An incubation step is performed under conditions of temperature, ionic strength, and pH that are permissive of antibody binding, and for a sufficient period of time to allow antibody-antigen binding. Antibody-antigen binding conditions and assay parameters are well known in the art.

Detection can be by an immunological assay, described in further detail in a later section, such as radioimmune assay or ELISA performed in any of a wide variety of formats, or by detecting binding on a solid support or semi-solid support using labeled reagents, which can be signal-generating reagents. Detection of binding of a biomarker to a solid support can be detection on or in a gel, matrix, filter, strip, sheet, strip, membrane, slide, plate (for example, a multiwell plane), well, dish, bead, particle, filament, rod, fiber, chip, or array. In some preferred embodiments, binding of autoantigens present in the sample to autoantibody capture molecules on a protein array is detected. The protein array can have proteins other than autoantibody capture molecules bound to the array, such as, but not limited to, antibodies that are used to detect proteins that are not necessarily bound to autoantibodies, negative or positive control proteins, proteins used for normalization of signal intensity, and proteins (including but not limited to antibodies) whose reactivity or binding status to a test sample is unknown.

The invention provides autoantibody capture molecules, such as target antigens and target antibodies, for detecting autoantibodies in a sample from an individual, methods for detecting cancer, such as prostate cancer, by detecting autoantibodies in an individual, and biomarker detection panels comprising combinations of the target antigens and/or target antibodies of Table 1 that can be used to detect and diagnose prostate cancer with high sensitivity and specificity. Biomarker detection panels can include sets of autoantibody capture molecules that have high sensitivity and specificity for detecting prostate cancer, including the biomarker detection sets provided in Table 5, Table 6, Table 7, Table 8, and Table 9. Target antigens for detecting autoantibodies present in samples of prostate cancer that were identified using the PROTOARRAY™ human protein microarray (Invitrogen, Carlsbad, Calif.) are also provided (Table 11a). Biomarker detection panels can include one or both of KDR, PIM-1, or variants or fragments thereof (Example 3). Accordingly, methods, compositions, and kits are provided herein for the detection, diagnosis, staging, and monitoring of cancer, such as prostate cancer, in individuals.

### DEFINITIONS

Unless defined otherwise, all technical and scientific terms used herein have the same meanings as commonly understood by one of ordinary skill in the art to which this invention belongs.

The term “about” as used herein refers to a value within 10% of the underlying parameter (i.e., plus or minus 10%), and is sometimes a value within 5% of the underlying parameter (i.e., plus or minus 5%), a value sometimes within 2.5% of the underlying parameter (i.e., plus or minus 2.5%), or a value sometimes within 1% of the underlying parameter (i.e., plus or minus 1%), and sometimes refers to the parameter with no variation. Thus, a distance of “about 20 nucleotides in length” includes a distance of 19 or 21 nucleotides in length (i.e., within a 5% variation) or a distance of 20 nucleotides in length (i.e., no variation) in some embodiments.

As used herein, the article “a” or “an” can refer to one or more of the elements it precedes (e.g., a protein microarray “a” protein may comprise one protein sequence or multiple proteins).

The term “or” is not meant to be exclusive to one or the terms it designates. For example, as it is used in a phrase of the structure “A or B” may denote A alone, B alone, or both A and B.

By “biomarker” is meant a biochemical characteristic that can be used to diagnose, or to measure the progress of a disease or condition, or the effects of treatment of a disease or condition. A biomarker can be, for example, the presence of a nucleic acid, protein, or antibody associated with the presence of cancer or another disease in an individual. The present invention provides biomarkers for prostate cancer that are antibodies present in the sera of subjects diagnosed with prostate cancer. The biomarker antibodies in the present invention are the autoantibodies displaying increased reactivity in individuals with prostate cancer, most likely as a consequence of their increased abundance. The autoantibodies can be detected with autoantigens, human proteins that are specifically bound by the antibodies. Established biomarkers vary widely in the frequency with which they are observed. Importantly, biomarkers need not be expressed in a majority of disease individuals to have clinical value. The receptor tyrosine kinase Her2 is known to be over-expressed in approximately 25% of all breast cancers (J. S. Ross et al., Mol Cell Proteomics 3, 379-98 (April, 2004)), and yet is a clinically important indicator of disease progression as well as specific therapeutic options.

“Biomolecule” refers to an organic molecule of biological origin, e.g., steroids, fatty acids, amino acids, nucleotides, sugars, peptides, polypeptides (proteins), antibodies, polynucleotides, complex carbohydrates or lipids.

As used herein, the word “protein” refers to a full-length protein, a portion of a protein, or a peptide. The term protein includes antibodies. Proteins can be produced via fragmentation of larger proteins, or chemically synthesized. Proteins may, for example, be prepared by recombinant overexpression in a species such as, but not limited to, bacteria, yeast, insect cells, and mammalian cells. Proteins to be placed in a protein microarray of the invention, may be, for example, are fusion proteins, for example with at least one affinity tag to aid in purification and/or immobilization. In certain aspects of the invention, at least 2 tags are present on the protein, one of which can be used to aid in purification and the other can be used to aid in immobilization. In certain illustrative aspects, the tag is a His tag, a FLAG tag, a GST tag, or a biotin tag. These examples are non-limiting. Where the tag is a biotin tag, the tag can be associated with a protein in vitro or in vivo using commercially available reagents (Invitrogen, Carlsbad, Calif.). In aspects where the tag is associated with the protein in vitro, a Bioease tag can be used (Invitrogen, Carlsbad, Calif.).

As used herein, the term “peptide,” “oligopeptide,” and “polypeptide” are used interchangeably with protein herein and refer to a sequence of contiguous amino acids linked by peptide bonds. As used herein, the term “protein” refers to a polypeptide that can also include post-translational modifications that include the modification of amino acids of the protein and may include the addition of chemical groups or biomolecules that are not amino acid-based. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

The term “antigen” or “test antigen” as used herein refers to proteins or polypeptides to be used as targets for screening test samples obtained from subjects for the presence of autoantibodies. “Autoantigen” is used to denote antigens for which the presence of antibodies in a sample of an individual has been detected. These antigens, test antigens, or autoantigens are contemplated to include any fragments thereof of the so-identified proteins, in particular, immunologically detectable fragments. The terms antigen and test antigen are also meant to include immunologically detectable products of proteolysis of the proteins, as well as processed forms, post-translationally modified forms, such as, for example, “pre” “pro,” or “prepro” forms of markers, or the “pre,” “pro,” or “prepro” fragment removed to form the mature marker, as well as sequence variants, including but not limited to allelic variants and splice variants of the antigens, test antigens, or autoantigens or fragments thereof. The identification or listing of antigens, test antigens, and autoantigens also includes amino acid sequence variants of these, for example, sequence variants that include a fragment, domain, or epitope that shares immune reactivity with the identified antigen, test antigen, and autoantigen protein. The fragment, domain, or epitope can be provided as part of or attached to a larger molecule or compound.

As used herein, “target antigen” refers to a protein, or to a portion, fragment, variant, isoform, processing product thereof having immunoreactivity of the protein, that is used to determine the presence, absence, or amount of an antibody in a sample from a subject. A “test antigen” is a protein evaluated for use as a target antigen. A test antigen is therefore a candidate target antigen, or a protein used to determine whether a portion of a test population has antibodies reactive against it. Use of the terms “target antigen”, “test antigen”, “autoantigen”, and, simply, “antigen” is meant to include the complete wild type mature protein, or can also denote a precursor, processed form (including, a proteolytically processed or otherwise cleaved form) unprocessed form, post-translationally modified, or chemically modified form of the protein indicated, in which the target antigen, test antigen, or antigen retains or possesses the specific binding characteristics of the referenced protein to one or more autoantibodies of a test sample. The protein can have, for example, one or more modifications not typically found in the protein produced by normal cells, including aberrant processing, cleavage or degradation, oxidation of amino acid residues, atypical glycosylation pattern, etc. The use of the terms “target antigen”, “test antigen”, “autoantigen”, or “antigen” also include splice isoforms or allelic variants of the referenced proteins, or can be sequence variants of the referenced protein, with the proviso that the “target antigen”, “test antigen”, “autoantigen”, or “antigen” retains or possesses the immunological reactivity of the referenced protein to one or more autoantibodies of a test sample. Use of the term “target antigen”, “test antigen”, “autoantigen”, or simply “antigen” specifically encompasses fragments of a referenced protein (“antigenic fragments”) that have the antibody binding specificity of the reference protein. The fragment can be provided as part of or attached to a larger molecule or compound.

An “autoantibody” is an antibody present in an individual that specifically recognizes a biomolecule present in the individual. Typically an autoantibody specifically binds a protein expressed by the individual, or a modified form thereof present in a sample from the individual. Autoantibodies are generally IgG antibodies that circulate in the blood of an individual, although the invention is not limited to IgG autoantibodies or to autoantibodies present in the blood.

An “autoantibody capture molecule” is a reagent that specifically binds a particular autoantibody, or an autoantigen-autoantibody complex, in a sample from an individual. An autoantibody capture molecule can be, for example, a protein (or target antigen) that can directly bind an autoantibody, or can be an antibody (for example a target antibody) that indirectly binds an autoantibody that is complexed with an autoantigen that can specifically bind to the target antibody.

The term “target antibody” is herein used to mean an antibody that can bind an antigen-autoantibody complex.

A “variant” of a polypeptide or protein, as used herein, refers to an amino acid sequence that is altered with respect to the referenced polypeptide or protein by one or more amino acids. In the present invention, a variant of a polypeptide retains the antibody-binding property of the referenced protein. In preferred aspects of the invention, a variant of a polypeptide or protein can be specifically bound by the same population of autoantibodies that are able to bind the referenced protein. Preferably a variant of a polypeptide has at least 60% identity to the referenced protein over a sequence of at least 10 amino acids. More preferably a variant of a polypeptide is at least 70% identical to the referenced protein over a sequence of at least 4 amino acids. Protein variants can be, for example, at least 80%, at least 90%, at least 95%, at least 96%, at least 97%, at least 98%, or at least 99% identical to referenced polypeptide over a sequence of at least 4 amino acids. Protein variants of the invention can be, for example, at least 80%, at least 90%, at least 95%, at least 96%, at least 97%, at least 98%, or at least 99% identical to referenced polypeptide over a sequence of at least 10 amino acids. The variant may have “conservative” changes, wherein a substituted amino acid has similar structural or chemical properties (e.g., replacement of leucine with isoleucine). A variant may also have “nonconservative” changes (e.g., replacement of glycine with tryptophan). Analogous minor variations may also include amino acid deletions or insertions, or both. Guidance in determining which amino acid residues may be substituted, inserted, or deleted without abolishing immunological reactivity may be found using computer programs well known in the art, for example, DNASTAR software.

As used herein, a “biomarker detection panel” or “biomarker panel” refers to a collection of biomarkers that are provided together for detection, diagnosis, prognosis, staging, or monitoring of a disease or condition, based on detection values for the set (panel) of biomarkers. The set of biomarkers is physically associated, such as by being packaged together, or by being reversibly or irreversibly bound to a solid support. For example, the biomarker detection panel can be provided, in separate tubes that are sold and/or shipped together, for example as part of a kit, or can be provided on a chip, membrane, strip, filter, or beads, particles, filaments, fibers, or other supports, in or on a gel or matrix, or bound to the wells of a multiwell plate. A biomarker detection panel can in addition or in the alternative be associated by a list, table, or program provided to a user or potential user that provides an internet address that provides computer-based linkage of the biomarker identities and information stored on a web site. A computer-based program can provide links between biomarker identities, information, and/or purchasing functions for a collection of biomarkers that make up a biomarker detection panel, based on the user's entered selections.

The phrase “differentially present” refers to differences in the quantity of a biomolecule (such as an antibody) present in a sample taken from patients having prostate cancer as compared to a comparable sample taken from patients who do not have prostate cancer (e.g., have benign prostate hyperplasia). A biomolecule is differentially present between the two samples if the amount of the polypeptide in one sample is significantly different from the amount of the polypeptide in the other sample. For example, a polypeptide is differentially present between the two samples if it is present in an amount (e.g., concentration, mass, molar amount, etc.) at least about 150%, at least about 200%, at least about 500% or at least about 1000% greater than it is present in the other sample, or if it is detectable (gives a signal significantly greater than background or a negative control) in one sample and not detectable in the other. Any biomolecules that are differentially present in samples taken from prostate cancer patients as compared to subjects who do not have prostate cancer (e.g., benign prostate hypertrophy patients) can be used as biomarkers.

A “sample” as used herein can be any type of sample, such as a sample of cells or tissue, or a sample of bodily fluid, preferably from an animal, most preferably a human. The sample can be a tissue sample, such as a swab or smear, or a pathology or biopsy sample of tissue, including tumor tissue. Samples can also be tissue extracts, for example from tissue biopsy or autopsy material. A sample can be a sample of bodily fluids, such as but not limited to blood, plasma, serum, cerebral-spinal-fluid, sputum, semen, urine, lung aspirates, nipple aspirates, tears, or a lavage. Samples can also include, for example, cells or tissue extracts such as homogenates or solubilized tissue obtained from a patient. A preferred sample is a blood or serum sample. By “blood” is meant to include whole blood, plasma, serum, or any derivative of blood. A blood sample may be, for example, serum.

A “patient” is an individual diagnosed with a disease or being tested for the presence of disease. A patient tested for a disease can have one or more indicators of a disease state, or can be screened for the presence of disease in the absence of any indicators of a disease state. As used herein an individual “suspected” of having a disease can have one or more indicators of a disease state or can be part of a population routinely screened for disease in the absence of any indicators of a disease state.

By “an individual suspected of having prostate cancer,” is meant an individual who has been diagnosed with prostate cancer, or who has at least one indicator of prostate cancer, including but not limited to, a prostate biopsy pathology report that states at least one of the biopsy cores contains carcinoma or adenocarcinoma and gives a Gleason score value, a PSA level of greater than 4 ng per ml, a PSA serum level of greater than 10 ng per mL, enlarged prostate, or a positive PCA3 test.

As used herein, the term “array” refers to an arrangement of entities in a pattern on a substrate. Although the pattern is typically a two-dimensional pattern, the pattern may also be a three-dimensional pattern. The individual entities are localized to particular positions, or loci on the array, sometimes referred to as “spots”. In a protein array, the entities are proteins. In certain embodiments, the array can be a microarray or a nanoarray. A “nanoarray” is an array in which separate entities are separated by 0.1 nm to 10 μm, for example from 1 nm to 1 μm. A “microarray” is an array in the density of entities on the array is at least 100 distinct loci/cm². A high density array has at least 400 distinct loci per cm². For example, a high density protein array has at least 400 distinct protein spots per cm². In some embodiments, a high density array has at least 1,000 distinct loci per cm². For example, a high density protein array has at least 1,000 distinct protein spots per cm². On microarrays separate entities can be separated, for example, by more than 1 μm.

The term “protein array” as used herein refers to a protein array, a protein microarray or a protein nanoarray. A protein array may include, for example, but is not limited to, a “ProtoArray™” human protein high density array (Invitrogen, Carlsbad, Calif., available on the Internet at Invitrogen.com)) The ProtoArray™ high density protein array can be used to screen complex biological mixtures, such as serum, to assay for the presence of autoantibodies directed against human proteins. Alternatively, a custom protein array that includes autoantigens, such as those provided herein, for the detection of autoantibody biomarkers, can be used to assay for the presence of autoantibodies directed against human proteins. In certain disease states including autoimmune diseases and cancer, autoantibodies are expressed at altered levels relative to those observed in healthy individuals.

The term “protein chip” is used in the present application synonymously with protein array or microarray.

The phrase “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, or amount of which is indicative of the presence, severity, or absence of the condition, physical features (lumps or hard areas in or on tissue), or histological or biochemical analysis of biopsied or sampled tissue or cells, or a combination of these.

Similarly, a prognosis is often determined by examining one or more “prognostic indicators”, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability of having a disease or condition in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. Preferred prognostic markers can predict the onset of prostate cancer in a patient with PIN, or a more advanced stage of prostate cancer in a patient diagnosed with prostate cancer.

The term “correlating,” as used herein in reference to the use of diagnostic and prognostic indicators, refers to comparing the presence or amount of the indicator in a patient to its presence or amount in persons known to suffer from, or known to be at risk of, a given condition; or in persons known to be free of a given condition. As discussed above, a marker level in a patient sample can be compared to a level known to be associated with prostate cancer. The sample's marker level is said to have been correlated with a diagnosis; that is, the skilled artisan can use the marker level to determine whether the patient has prostate cancer, and respond accordingly. Alternatively, the sample's marker level can be compared to a marker level known to be associated with a good outcome (e.g., the absence of prostate cancer, etc.). In preferred embodiments, a profile of marker levels are correlated to a global probability or a particular outcome using ROC curves.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. For example, in individuals not exhibiting the condition, the chance of a given outcome may be about 3%. In preferred embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

“Diagnosis” means identifying the presence or nature of a pathologic condition. Diagnostic methods differ in their sensitivity and specificity. While a particular diagnostic method may not provide a definitive diagnosis of a condition, it suffices if the method provides a positive indication that aids in diagnosis.

“Sensitivity” is defined as the percent of diseased individuals (individuals with prostate cancer) in which the biomarker of interest is detected (true positive number/total number of diseased×100). Nondiseased individuals diagnosed by the test as diseased are “false positives”.

“Specificity” is defined as the percent of nondiseased individuals for which the biomarker of interest is not detected (true negative/total number without disease×100). Diseased individuals not detected by the assay are “false negatives.” Subjects who are not diseased and who test negative in the assay, are termed “true negatives.”

A “diagnostic amount” of a marker refers to an amount of a marker in a subject's sample that is consistent with a diagnosis of prostate cancer. A diagnostic amount can be either in absolute amount (e.g., X nanogram/ml) or a relative amount (e.g. relative intensity of signals).

A “test amount” of a marker refers to an amount of a marker present in a sample being tested. A test amount can be either in absolute amount (e.g., X nanogram/ml) or a relative amount (e.g., relative intensity of signals).

A “control amount” of a marker can be any amount or a range of amount which is to be compared against a test amount of a marker. For example, a control amount of a marker can be the amount of a marker (e.g., seminal basic protein) in a prostate cancer patient, a BPH patient or a person without prostate cancer or BPH. A control amount can be either in absolute amount (e.g., X nanogram/ml) or a relative amount (e.g., relative intensity of signals).

“Detect” refers to identifying the presence, absence or relative or absolute amount of the object to be detected.

“Label” or a “detectable moiety” refers to a composition detectable by spectroscopic, photochemical, biochemical, immunochemical, electromagnetic, or chemical means. For example, useful labels include radiolabels such as ³²P, ³⁵S, or ¹²⁵I; heavy isotopes such as ¹⁵N or ¹³C or heavy atoms such as selenium or metals; fluorescent dyes; chromophores, electron-dense reagents; enzymes that generate a detectable signal (e.g., alkaline phosphatase or peroxidase, as commonly used in an ELISA); or spin labels. The label or detectable moiety has or generates a measurable signal, such as a radioactive, chromogenic, or fluorescent signal, that can be used to quantify the amount of bound detectable moiety in a sample. The detectable moiety can be incorporated in or attached to a molecule (such as a protein, for example, an antibody) either covalently, or through ionic, van der Waals or hydrogen bonds, e.g., or by incorporation of labeled precursors. The label or detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavidin, which can be linked to a directly detectable label. The binding partner may itself be directly detectable, for example, an antibody may be itself labeled with a fluorescent molecule. The binding partner also may be indirectly detectable, for example, it may be bound by another moiety that comprises a label. Quantitation of the signal is achieved by any appropriate means, e.g., fluorescence detection, spectrophotometric detection (e.g., absorption at a particular wavelength), scintillation counting, mass spectrometry, densitometry, or flow cytometry.

“Measure” in all of its grammatical forms, refers to detecting, quantifying or qualifying the amount (including molar amount), concentration or mass of a physical entity or chemical composition either in absolute terms in the case of quantifying, or in terms relative to a comparable physical entity or chemical composition.

“Antibody” refers to a polypeptide ligand substantially encoded by an immunoglobulin gene or immunoglobulin genes, or fragments thereof, which specifically recognizes and binds a molecule or a region or domain of a molecule (an epitope). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

An “antibody to a protein” or an “antibody that recognizes a protein” is an antibody that specifically binds the protein.

“Immunoassay” is an assay in which an antibody specifically binds an antigen. An immunoassay is characterized by the use of specific binding properties of a particular antibody to isolate, capture, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at a level that is statistically significantly different from background, and do not substantially bind in a significant amount to other proteins present in the sample. In methods of the invention in which an antibody is used as a capture molecule, the antibody is selected for its specificity for a particular protein, and also, in some embodiments, for its ability to specifically bind an antigen that is complexed with an autoantibody. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). In some embodiments, a specific or selective reaction will be at least twice background signal or noise and more typically more than five times the background signal, and can be, for example, 10 to 100 times background.

“Immune reactivity” as used herein means the presence or level of binding of an antibody or antibodies in a sample to one or more target antigens. A “pattern of immune reactivity” refers to the profile of binding of antibodies in a sample (autoantibodies) to a plurality of target antigens and/or target antibodies. The profile includes the subset of target antigens and/or target antibodies to which the sample specifically binds, and/or the relative or absolute level(s) of binding to members of the subset of target antigens and/or target antibodies to which binding is detected.

An “epitope” is a site on an antigen, such as an autoantigen disclosed herein, recognized by an antibody.

### Methods

The invention provides, in one aspect, a method of detecting an autoantibody in a sample from an individual. The method includes: contacting a sample from the individual with an autoantibody capture molecule of the invention, and detecting binding of an antibody in the sample to the autoantibody capture molecule, thereby detecting an autoantibody in the individual.

The autoantibody capture molecule can be a target antigen that can specifically bind an autoantibody, or can be an antibody that can specifically bind an autoantigen that is complexed with an autoantibody. The autoantibody capture molecule can be any of the target antigens or target antibodies provided in Table 1, or can be an antibody to a protein of Table 1 that can bind an antigen-autoantibody complex; and in some preferred embodiments is a protein of Table 2, or is an antibody that recognizes a protein of Table 2, in which the antibody can bind a target antigen of Table 2 that is complexed with an autoantibody in the sample. Target antigens specifically include variants and modified forms of the proteins listed in Table 2, and specifically include epitope-containing fragments of the proteins listed in Table 2. In some exemplary embodiments, the autoantibody capture molecule used in the methods is HEYL, MLH1, BDKRB2, PTGER3, RPL30, ZWINT, BIRC5, TOP2A, AZGP1, CLDN3, MAD1L1, PRSS8, PSAP, PSMAB4, QSCN6, RPS6KA1, SPRR1B, EIF3S3, CCNA1, RNF14, CD151, NCAM2, ETS2, MICB, NUCB1, COVA1, RASSF1, STEAP, NRP1, SH3GLB1, RDH11, BCLG, CCNB1, CCND1, or EIF4G1, or is an antibody to HEYL, MLH1, BDKRB2, PTGER3, RPL30, ZWINT, BIRC5, TOP2A, AZGP1, CLDN3, MAD1L1, PRSS8, PSAP, PSMAB4, QSCN6, RPS6KA1, SPRR1B, EIF3S3, CCNA1, RNF14, CD151, NCAM2, ETS2, MICB, NUCB1, COVA1, RASSF1, STEAP, NRP1, SH3GLB1, RDH11, BCLG, CCNB1, CCND1, or EIF4G1, in which the antibody can bind an autoantigen-autoantibody complex. In some exemplary embodiments, the autoantibody capture molecule is HEYL, BDKRB2, PTGER3, ZWINT, TOP2A, PSAP, RPS6KA1, SPRR1B, CCNA1, RNF14, NCAM2, ETS2, COVA1, RASSF1, SH3GLB1, CCNB1, or EIF4G1, or is an antibody to HEYL, BDKRB2, PTGER3, ZWINT, TOP2A, PSAP, RPS6KA1, SPRR1B, CCNA1, RNF14, NCAM2, ETS2, COVA1, RASSF1, SH3GLB1, CCNB1, or EIF4G1 in which the antibody can bind an autoantigen-autoantibody complex.

In some exemplary embodiments, the autoantibody capture molecule used in the methods is KDR or PIM-1, or is an antibody to KDR or PIM-1 in which the antibody can bind an autoantigen-autoantibody complex.

In the methods provided herein, the sample can be any sample of cells or tissue (including extracts thereof), or of any bodily fluid. Since the autoantibodies being screened for circulate in the blood and are fairly stable in blood samples, in certain illustrative embodiments, the test sample is blood or a fraction thereof, such as, for example, serum. The sample can be unprocessed prior to contact with the test antigen, or can be a sample that has undergone one or more processing steps. For example, a blood sample can be processed to remove red blood cells and obtain serum.

The individual from whom the test sample is taken can be any individual, and in some embodiments is an individual that is being screened for cancer. Autoantibodies detected in a sample from an individual can be indicative of more than one type of cancer. Individuals testing positive for autoantibodies indicative of more than one type of cancer can be further screened to determine whether a given type of cancer is present in the individual. In some embodiments, an individual from whom a sample is taken can be an individual being screened for any of prostate, breast, liver, ovarian, pancreatic, uterine, stomach, bone, brain, colorectal, bladder, or lung cancer, or a leukemia or lymphoma.

In some embodiments, the individual from whom a sample is taken for contacting with a target antigen of the invention can be a male individual being screened for prostate cancer. In some of these embodiments, the method includes: contacting a sample from the individual with a protein of Table 4, or an antibody to a protein of Table 4, and detecting binding of an antibody in the sample to the protein of Table 4, or binding of an antigen-antibody complex of the sample to the antibody to a protein of Table 4. The method can be used to detect, diagnose, prognose, stage, or monitor prostate cancer or prostate intraepithelial neoplasia (PIN) in an individual. In some embodiments, the method is used to distinguish prostate cancer from BPH. In some exemplary embodiments, the method includes contacting a sample from an individual with one or more of the target antigens: HEYL, MLH1, PTEN, BDKRB2, BCL2, PTGER3, RPL30, ZWINT, ERBB2, BIRC5, TOP2A, ACPP, AZGP1, CLDN3, HSPB1, CAV3, HSPD1, KDR, MAD1L1, PRSS8, PSAP, PSMB4, QSCN6, RPS6KA1, SPRR1B, TRA1, HMGA2, EIF3S3, CCNA1, RNF14, CD151, NCAM2, EGFR, ETS2, HSPA1A, MICB, CD164, NUCB1, COVA1, IMP-3, STIP1, RASSF1, STEAP, NRP1, SH3GLB1, RDH11, XLKD1, BCLG, CCNB1, CCND1, PCNA, or EIF4G1 or with one or more antibodies to HEYL, MLH1, PTEN, BDKRB2, BCL2, PTGER3, RPL30, ZWINT, ERBB2, BIRC5, TOP2A, ACPP, AZGP1, CLDN3, HSPB1, CAV3, HSPD1, KDR, MAD1L1, PRSS8, PSAP, PSMB4, QSCN6, RPS6KA1, SPRR1B, TRA1, HMGA2, EIF3S3, CCNA1, RNF14, CD151, NCAM2, EGFR, ETS2, HSPA1A, MICB, CD164, NUCB1, COVA1, IMP-3, STIP1, RASSF1, STEAP, NRP1, SH3GLB1, RDH11, XLKD1, BCLG, CCNB1, CCND1, PCNA, or EIF4G1; and detecting an autoantibody to one or more of the target antigens or antibodies to target antigens to detect, diagnose, monitor, stage, or prognose prostate cancer or PIN in an individual. In some exemplary embodiments, the method includes contacting a sample from an individual with one or more of the target antigens: HEYL, BDKRB2, PTGER3, ZWINT, TOP2A, PSAP, RPS6KA1, SPRR1B, HMGA2, CCNA1, RNF14, NCAM2, ETS2, CD164, COVA1, RASSF1, SH3GLB1, XLKD1, CCNB1, PCNA, ERBB2, or EIF4G1 or with one or more antibodies to one or more of these target antigens (HEYL, BDKRB2, PTGER3, ZWINT, TOP2A, PSAP, RPS6KA1, SPRR1B, HMGA2, CCNA1, RNF14, NCAM2, ETS2, CD164, COVA1, RASSF1, SH3GLB1, XLKD1, CCNB1, PCNA, ERBB2, or EIF4G1); and detecting an autoantibody to one or more of the target antigens to detect, diagnose, monitor, stage, or prognose prostate cancer or PIN in an individual. In some exemplary embodiments, the method includes contacting a sample from an individual with one or more of the target antigens: HEYL, PTGER3, ZWINT, SPRR1B, SH3GLB1, RPL30, PSMB4, MICIB, IMP-3, or CCNB1 or with one or more antibodies to one or more of the target antigens (HEYL, PTGER3, ZWINT, SPRR1B, SH3GLB1, RPL30, PSMB4, MICIB, IMP-3, or CCNB1), and detecting an autoantibody in the sample to one or more of HEYL, PTGER3, ZWINT, SPRR1B, SH3GLB1, RPL30, PSMB4, MICIB, IMP-3, or CCNB1 to detect, diagnose, monitor, stage, or prognose prostate cancer or PIN in an individual. In some exemplary embodiments, the method includes contacting a sample from an individual with one or more of the target antigens: HEYL, PTGER3, ZWINT, SPRR1B, SH3GLB1, or CCNB1 or with an antibody to one or more of the target antigens HEYL, PTGER3, ZWINT, SPRR1B, SH3GLB1, or CCNB1; and detecting an autoantibody in the sample to one or more of HEYL, PTGER3, ZWINT, SPRR1B, SH3GLB1, or CCNB1 to detect, diagnose, monitor, stage, or prognose prostate cancer or PIN in an individual.

The individual from which the sample is taken can be, for example, a male individual that can have one or more risk factors for prostate cancer, including but not limited to, being of age 50 or older, being of African heritage, or having a family history of prostate cancer. The individual can have one or more indicators of prostate cancer. For example, the individual can be an individual with enlarged prostate. The individual can be a male with a PSA level of greater than 4 ng per ml, or greater than 10 ng per ml. The individual can also be a male with a PSA level of less than 4 ng per ml, such as between 0 and 4 ng per ml. The individual can be a male with a ratio of free to total PSA of less than 0.25. The individual can be a male with a level of PCA3 transcript that is suggestive of prostate cancer or BPH. The individual can be an individual previously diagnosed with PIN. The individual can also be an individual who has previously been tested or biopsied for prostate cancer and found to be negative.

The test sample can be contacted with an autoantibody capture molecule provided in solution phase, or the autoantibody capture molecule can be provided bound to a solid support. The sample can be diluted or concentrated, or subjected to one or more processing steps prior to contacting with an autoantibody capture molecule. After contacting the sample with the autoantibody capture molecule, incubation is performed under conditions of temperature, ionic strength, and pH that are permissive of antibody binding, for a sufficient period of time to allow antibody-antigen binding. Antibody-antigen binding conditions and assay parameters are well known in the art. Detection of autoantibodies can be performed using an immunoassay, which can be in any of various formats that include that detection of proteins by antibodies, including those described in further detail below.

Detection of binding in certain illustrative embodiments makes use of one or more solid or semi-solid supports to which the autoantibody capture molecule is immobilized and to which the sample from an individual, in preferred embodiments a human subject, is applied. In exemplary embodiments, after incubation of the sample with the immobilized autoantibody capture molecule (and, preferably, subsequent wash steps), or optionally, concurrently with the incubation of the sample and autoantibody capture molecule, an antibody that is reactive against antibodies of the species from which the sample is taken, e.g., anti-human antibodies (for example, an anti-human IgG antibody that is from a species other than human, for example, goat, rabbit, pig, mouse, etc.) can be applied to the solid or semi-solid support with which the sample is incubated. The anti-human IgG antibody is directly or indirectly labeled. In some embodiments, the anti-human IgG antibody is labeled in one or more additional steps after the anti-human IgG antibody is contacted with the immobilized antigen that has been contacted with a sample. After removing nonspecifically bound antibody, signal from the label that is significantly above background level is indicative of binding of a human antibody from the sample to an autoantibody capture molecule on the solid or semi-solid support.

Detection antibodies, such as for example anti-species-specific antibodies can be used to detect a captured autoantibody bound directly to a target antigen or can be used to detect a captured autoantibody bound indirectly to a target antibody. Autoantibodies bound indirectly to a target antibody are autoantibodies that are captured as autoantibody-autoantigen complexes.

The invention thus in some aspects provides methods of detecting an autoantibody in a sample from an individual, in which the method includes: contacting a sample from an individual with at least one target antibody, in which the target antibody specifically binds an antigen of Table 2 or Table 4, and detecting binding of an autoantibody-autoantigen complex of the sample to the target antibody to detect an autoantibody in the sample. A target antibody can be any antibody that specifically binds to an antigen of Table 2 or Table 4, and in some exemplary embodiments is an antibody to HEYL, MLH1, BDKRB2, PTGER3, RPL30, ZWINT, BIRC5, TOP2A, AZGP1, CLDN3, MAD1L1, PRSS8, PSAP, PSMAB4, QSCN6, RPS6KA1, SPRR1B, EIF3S3, CCNA1, RNF14, CD151, NCAM2, ETS2, MICB, NUCB1, COVA1, RASSF1, STEAP, NRP1, SH3GLB1, RDH11, BCLG, CCNB1, CCND1, or EIF4G1.

Detection of binding of the autoantibody to a target antibody is by detecting binding of an autoantigen-autoantibody complex to a target antibody. Detection of binding can be by any means that detects the autoantibody. The detection preferably uses anti-species-specific antibodies. For example, where the sample is from a human, the method can include detecting an autoantibody by detecting binding of an anti-human IgG antibody to the autoantigen-autoantibody complex bound to the target antibody. The anti-species specific IgG antibodies can be directly or indirectly labeled.

The detection of autoantibodies bound by either target antigens or target antibodies can in some preferred embodiments be performed on a solid or semi-solid support of any type, such as a bead, particle, matrix, gel, filament, fiber, rod, dish, plate, well, sheet, membrane, slide, chip, or array, such as a protein array, which can be a microarray, and can optionally be a high density microarray. Analysis on the high density PROTOARRAY™ human protein microarray (Invitrogen, Carlsbad, Calif.) is shown in Example 2, and markers of the array correlating positively with prostate cancer are provided in Table 11a.

The detection method can provide a positive/negative binding result, or can give a value that can be a relative or absolute value for the level of the autoantibody biomarker in the sample. For example, detection of binding of the target sample to a test antigen indicates the presence of an autoantibody that specifically binds the test antigen in the sample. Identifying an autoantibody present in a sample from an individual can be used to identify biomarkers of a disease or condition, or to diagnose a disease or condition. In other embodiments, the level or levels of one or more autoantibodies can be detected using the methods of the invention. An increased or decreased level of an autoantibody that is detected in the assay in exemplary embodiments is a signal of at least two, and in some embodiments at least three, standard deviations above or below the signal level when a normal control (for example, serum from an individual who does not have cancer) is assayed. For example, autoantibodies can be detected in prostate cancer samples with an increased signal or a decreased signal when compared with BPH samples. The value of the autoantibody level in some exemplary embodiments can be detected as above or below a threshold value. The autoantibody level value is preferably normalized with respect to one or more controls provided in, alongside (in parallel with, preferably, or in tandem with) the detection assay. The result can provide a diagnosis or prognosis, or can be used as an indicator for conducting further tests or evaluation that may or may not result in a diagnosis or prognosis.

In certain illustrative embodiments, the methods provided herein for detecting autoantibodies are used to detect, diagnose, prognose, stage, monitor a disease state, pre-disease state, or medical condition. For example, the methods can be used to detect, diagnose, prognose, stage, or monitor cancer or a pre-cancerous state, such as but not limited to prostate cancer or PIN. In certain illustrative embodiments, the methods provided herein for detecting autoantibodies are used for managing a disease state, pre-disease state, or medical condition, including managing treatment regimes, or contributing to the decision of whether to treat a disease, pre-disease state, or medical condition, for example, pharmaceutically, with radiation therapy, surgically, or a combination of any of these. In some embodiments the methods can be used to test for recurrence of cancer after remission or anti-cancer treatment, such as but not limited to surgery or chemotherapy.

In some embodiments, the method includes detecting an autoantibody using an autoantibody capturing molecule from Table 2 or Table 4 in a sample from an individual and detecting one or more additional biomarkers for cancer in a sample from the same individual, in which the cancer can be any kind of cancer, including but not limited to: lung, liver, breast, uterine, ovarian, pancreatic, colorectal, stomach, esophageal, head and neck, brain, bone, or prostate cancer, or a lymphoma or leukemia. The additional biomarker for cancer can be of any type, for example, a protein, peptide, antibody, nucleic acid, hormone, growth factor, or metabolic marker, and detection can provide a positive or negative result, or can be detection of a relative or absolute value. The method can be used to detect, diagnose, stage, monitor, or prognose cancer in the individual. In some preferred embodiments, the method distinguishes between prostate cancer and BPH in an individual.

The detection and diagnostic methods of the invention can be repeated over time for the individual. For example, the individual can be an individual diagnosed with PIN that is being monitored for the presence of prostate cancer. The individual can be an individual diagnosed with prostate cancer in its early stages or with a low or intermediate Gleason score.

The individual can in some embodiments of the invention be an individual who has been treated or is being treated for prostate cancer, for example, using one or more pharmaceuticals, “nutriceuticals” or dietary regime, chemical anticancer agents, radiation therapy, or surgery. The results of a diagnostic test that determines the immune reactivity of a patient sample to a test antigen can be compared with the results of the same diagnostic test done at an earlier time. Significant differences in immune reactivity over time can contribute to a diagnosis or prognosis of prostate cancer. In these aspects, the methods and compositions provided herein can be used to detect regression, progression, or recurrence of cancer, such as prostate cancer.

The methods of the invention also include detecting two or more autoantibodies in a sample from an individual that bind to two or more target antigens, in which at least one of the two or more target antigens used to detect autoantibodies is a target antigen of Table 2 or Table 4, or an epitope-containing fragment thereof. In some embodiments, the method includes: contacting a sample from the individual with a plurality of autoantibody capture molecules of the invention, and detecting binding of an antibody in the sample to at least one of the plurality of autoantibody capture molecules, thereby detecting an autoantibody in the individual. In some embodiments, the method includes: contacting a sample from the individual with a plurality of target autoantibody capture molecules of the invention, and detecting binding of at least two autoantibodies in the sample to at least two of the plurality of autoantibody capture molecules, thereby detecting at least two autoantibodies in the individual. In exemplary embodiments, at least one of the plurality of target antigens used to detect autoantibodies in the individual is an autoantibody capture molecule of Table 2 or Table 4. One or more additional autoantibody capture molecules can be from Table 1 or Table 3. In some exemplary embodiments, at least two of the plurality of target antigens used to detect autoantibodies in the individual are target antigens of Table 2 or Table 4.

In one study described in Example 3, 96 selected proteins, hypothesized to be linked to prostate cancer (PCa) progression, were expressed, purified and then printed onto nitrocellulose slides. Each printed protein spot only contained approximately 0.03 picograms of protein, so cell free expression of approximately one to ten micrograms of protein was sufficient for even an extended protein array based study. Many of these proteins were subsequently revealed to have increased levels of autoantibodies correlating with PCa, and were not present in patients with benign prostatic hyperplasia (BPH).

This study revealed twenty antigens, including KDR and PIM-1 that are listed in Table 2 and Table 4, that induced significant humoral autoantibody response. Further studies with a low content protein chip showed that autoantibodies against KDR and PIM-1 differentiated prostate cancer from benign prostatic hyperplasia with 90.6% sensitivity and 84.4% specificity in thirty-two prostate cancer and thirty-two benign prostatic hyperplasia patients. Protein array signals were specific, and could be competed away by spiking pure antigen into the sera in a dose dependent manner. Additionally fluorescence immunohistochemistry of prostate cancer tissue arrays showed that KDR and PIM-1 were differentially expressed in prostate cancer tissues with reduced expression in benign prostatic hyperplasia tissues, suggesting over-expression of KDR and PIM-1 tumor antigens lead to the aberrant humoral response.

The use of a single autoantibody marker, KDR or PIM-1 alone, gave specificities of 62.5% and 65.6% respectively, while the combination of these two markers gave significant higher specificity of 84.4%. KDR autoantibody was present in ˜62% of the prostate cancer patient population, a significantly higher percentage than reported for some other tumor antigens. Since autoimmunity involves a polyclonal antibody response, using a truncated KDR may have exposed more epitopes for autoantibody capture, thereby yielding a higher frequency of positive responses in the cancer patient population.

The methods of the invention include detecting autoantibodies in a sample from an individual that bind to KDR and PIM-1, or an epitope-containing fragment thereof. In some embodiments, the method includes: contacting a sample from the individual with a plurality of autoantibody capture molecules of the invention, and detecting binding of an antibody that in the sample to at least one of KDR or PIM-1, or a fragment or variant thereof, thereby detecting an autoantibody to KDR or PIM-1 in the individual. In some embodiments, the method includes: contacting a sample from the individual with a plurality of target autoantibody capture molecules of the invention, and detecting binding of at least two autoantibodies in the sample to KDR or PIM-1, or a fragment or variant thereof, thereby detecting an autoantibody to KDR or PIM-1 in the individual. In some embodiments, the method is a method for diagnosing prostate cancer in an individual. In some embodiments, the method is a method for distinguishing prostate cancer from BHP in an individual

Another aspect of the invention is a method of diagnosing prostate cancer in an individual by contacting a sample from an individual with at least one autoantibody capture molecule that is either an antigen of Table 11a or an antibody that specifically binds an antigen of Table 11a, in which the antigen of Table 11a is complexed with an autoantibody, and detecting binding of the autoantibody capture molecule with at least one autoantibody of the sample, in which binding of the autoantibody capture molecule to an autoantibody of the sample is indicative of prostate cancer. In some embodiments, the method includes contacting a sample from the individual with two or more autoantibody capture molecules that are either antigens or antibodies that specifically bind, directly or indirectly, autoantibodies to the antigens of Table 11a that are present in a sample, and detecting binding of the sample to at least one of the one or more antigens or one or more antibodies to an autoantibody, in which binding of the sample to an antigen or Table 11a or an antibody to an antigen of Table 11a is indicative of prostate cancer. The method can include detecting binding of at least 2, at least 3, at least 4, at least 5, at least 6, at least 7, at least 8, at least 9, at least 10, between 10 and 15, between 15 and 20, between 20 and 25, between 25 and 30, between 30 and 35, between 35 and 40, between 40 and 45, between 45 and 50, between 50 and 60, between 60 and 70, between 70 and 80, between 80 and 90, or between 90 and 98 autoantibodies to antigens of Table 11a or to antibodies to the antigens listed in Table 11a. Antigens of Table 11a include variants and modified forms of the proteins listed in Table 11a, and include epitope-containing fragments of the proteins of Table 11a.

The methods can be used to diagnose, prognose, monitor, or stage prostate cancer or PIN. As demonstrated in Example 2 (discussed below), the methods can be used to distinguish High Grade and Low Grade prostate cancer. The methods can be used to distinguish prostate cancer from BPH.

In another aspect of the invention, the invention provides a method for detecting, diagnosing, staging, prognosing, and/or monitoring prostate cancer or PIN, that includes determining the pattern of immune reactivity of a sample from an individual to a biomarker detection panel that includes two or more autoantibody capture molecules of Table 1, in which one or more of the autoantibody capture molecules is from Table 3.

Autoantibody capture molecules include target antigens (including variants and modified forms thereof, and including epitope-containing fragments thereof), as well as target antibodies that can detect autoantibodies in a sample from an individual, such as autoantibodies present in a sample as autoantibody-autoantigen complexes. In these methods, the target antibodies can be those designated as antibodies in the tables provided herein, or can be antibodies to any of the designated target antigens (for example, antibodies to any of the target antigens of Table 1 or Table 3), in which the target antibodies specifically bind an antigen, in which the antigen is complexed with an autoantibody. The method includes: contacting a test sample from an individual with a biomarker detection panel that comprises two or more autoantibody capture molecules of Table 1, in which at least one of the autoantibody capture molecules is from Table 3; and determining the pattern of immune reactivity to the biomarker detection panel to diagnose, stage, prognose, or monitor prostate cancer. In preferred embodiments, the sample is a blood sample or a sample derived from a blood sample, such as serum.

The invention therefore includes in some aspects methods of diagnosing prostate cancer by contacting a sample of an individual with a biomarker detection panel that includes two or more autoantibody capture molecules of Table 1, in which at least one of the autoantibody capture molecules is a target antigen and at least one of the autoantibody capture molecules is a target antibody, and detecting at least one autoantibody bound to the target antigen and at least one autoantibody bound to the target antibody. In some preferred embodiments, at least one of the two auto or more autoantibody capture molecules is an autoantibody capture molecule of Table 3. In these methods, an autoantibody of a sample that is bound to a target antibody of a biomarker detection panel as an antigen-autoantibody complex and an autoantibody directly bound to a target antigen can both be detected using directly or indirectly labeled anti-species-specific antibodies, such as, for example, anti-species-specific IgG antibodies, such as anti-human IgG antibodies. The autoantibody capture molecules of the panel can optionally be bound to a common solid support, such as a protein array, such that in exemplary embodiments the addition of anti-species specific antibodies for detection of autoantibodies bound to both target antigen(s) and target antibody(ies) can be performed in a single step.

In some embodiments, the biomarker detection panel used in the methods of the invention includes 3, 4, 5, 6, 7, or 8 autoantibody capture molecules of Table 1. In some embodiments, the biomarker detection panel used in the methods of the invention includes 9, 10, 11, 12, 13, 14, 15, 16, 17, 18, 19, 20, or more autoantibody capture molecules of Table 1. In some embodiments, the test sample is contacted with a biomarker detection panel comprising 21, 22, 23, 24, 25, 26, 27, 28, 29, or 30 autoantibody capture molecules of Table 1. A biomarker detection panel can comprise between 30 and 35 autoantibody capture molecules of Table 1, between 35 and 40 autoantibody capture molecules of Table 1, between 40 and 45 autoantibody capture molecules of Table 1, between 45 and 50 autoantibody capture molecules of Table 1, between 50 and 55 autoantibody capture molecules of Table 1, between 55 and 60 autoantibody capture molecules of Table 1, between 60 and 65 autoantibody capture molecules of Table 1, between 65 and 70 autoantibody capture molecules of Table 1, between 70 and 75 autoantibody capture molecules of Table 1, between 75 and 80 autoantibody capture molecules of Table 1, between 80 and 85 autoantibody capture molecules of Table 1, between 85 and 90 autoantibody capture molecules of Table 1, between 90 and 95 autoantibody capture molecules of Table 1, between 95 and 100 autoantibody capture molecules of Table 1, between 100 and 110 autoantibody capture molecules of Table 1, or between 110 and 116 autoantibody capture molecules of Table 1. In all of these embodiments, one or more of the autoantibody capture molecules of Table 1 present in the biomarker detection panel can be an autoantibody capture molecule of Table 3.

In some embodiments, the biomarker detection panel used in the methods comprises two or more autoantibody capture molecules of Table 3. A biomarker detection panel can comprise, as nonlimiting examples, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 40-45, 45-50, 50-55, 55-60, 60-65, or 65-70 autoantibody capture molecules of Table 3.

In some preferred embodiments, one or more autoantibody capture molecules of a biomarker panel used in the methods is a protein of Table 2. In some embodiments, one or more autoantibody capture molecules is a protein of Table 4. In some embodiments, prostate cancer is diagnosed using a biomarker panel that comprises one or more of: HEYL, MLH1, PTEN, BDKRB2, BCL2, PTGER3, RPL30, ZWINT, ERBB2, BIRC5, TOP2A, ACPP, AZGP1, CLDN3, HSPB1, CAV3, HSPD1, KDR, MAD1L1, PRSS8, PSAP, PSMB4, QSCN6, RPS6KA1, SPRR1B, TRA1, HMGA2, EIF3S3, CCNA1, RNF14, CD151, NCAM2, EGFR, ETS2, HSPA1A, MICB, CD164, NUCB1, COVA1, IMP-3, STIP1, RASSF1,STEAP, NRP1, SH3GLB1, RDH11, XLKD1, BCLG, CCNB1, CCND1, PCNA, and EIF4G1, or an antibody to one or more of: HEYL, MLH1, PTEN, BDKRB2, BCL2, PTGER3, RPL30, ZWINT, ERBB2, BIRC5, TOP2A, ACPP, AZGP1, CLDN3, HSPB1, CAV3, HSPD1, KDR, MAD1L1, PRSS8, PSAP, PSMB4, QSCN6, RPS6KA1, SPRR1B, TRA1, HMGA2, EIF3S3, CCNA1, RNF14, CD151, NCAM2, EGFR, ETS2, HSPA1A, MICB, CD164, NUCB1, COVA1, IMP-3, STIP1, RASSF1,STEAP, NRP1, SH3GLB1, RDH11, XLKD1, BCLG, CCNB1, CCND1, PCNA, and EIF4G1. In some embodiments, prostate cancer is diagnosed using a biomarker panel that comprises on or more of: HEYL, BDKRB2, PTGER3, ZWINT, TOP2A, PSAP, RPS6KA1, SPRR1B, HMGA2, CCNA1, RNF14, NCAM2, ETS2, CD164, COVA1, RASSF1, SH3GLB1, XLKD1, CCNB1, PCNA, ERBB2, and EIF4G1, or an antibody to one or more of HEYL, BDKRB2, PTGER3, ZWINT, TOP2A, PSAP, RPS6KA1, SPRR1B, HMGA2, CCNA1, RNF14, NCAM2, ETS2, CD164, COVA1, RASSF1, SH3GLB1, XLKD1, CCNB1, PCNA, ERBB2, and EIF4G1. In some exemplary embodiments, prostate cancer is diagnosed using a biomarker panel that comprises on or more of: HEYL, PTGER3, ZWINT, SPRR1B, SH3GLB1, RPL30, PSMB4, MICIB, IMP-3, or CCNB1; or comprises an antibody to one or more of HEYL, PTGER3, ZWINT, SPRR1B, SH3GLB1, RPL30, PSMB4, MICIB, IMP-3, and CCNB1. In some exemplary embodiments, prostate cancer is diagnosed using a biomarker panel that comprises one or more of: HEYL, PTGER3, ZWINT, SPRR1B, SH3GLB1, and CCNB1; or comprises one or more of HEYL, PTGER3, ZWINT, SPRR1B, SH3GLB1, and CCNB1.

In some embodiments, the biomarker detection panel used in the methods of the invention has an ROC/AUC value of 0.800 or greater, or 0.850 or greater. In some embodiments, the biomarker detection panel has an ROC/AUC value of 0.950 or greater.

In some embodiments, the biomarker detection panel used in the methods that comprises two or more autoantibody capture molecules of Table 1 comprises one or more autoantibody capture molecules of Table 10. In some exemplary embodiments, a biomarker detection panel used in the methods of the invention comprises two or more autoantibody capture molecules of Table 3, in which at least one of the two or more autoantibody capture molecules of Table 3 is an autoantibody capture molecule of Table 10.

In some embodiments, the invention includes methods for detecting, diagnosing, staging, prognosing, and/or monitoring prostate cancer or PIN, or a method for differentiating BPH from prostate cancer in an individual, that includes determining the immune reactivity of a test sample from the individual against a biomarker detection panel that includes sets of autoantibody capture molecules (“autoantibody detection sets”) that when used together have a high specificity and sensitivity for diagnosing prostate cancer and for distinguishing prostate cancer from BPH. The autoantibody detection sets can in some embodiments comprise one or more autoantibody capture molecules of Table 10.

In exemplary embodiments, the invention includes methods for detecting, diagnosing, staging, prognosing, and/or monitoring prostate cancer, or a method for differentiating BPH from prostate cancer in an individual, that includes determining the immune reactivity of a test sample from the individual against a biomarker detection panel that comprises a 3-entity autoantibody detection set of Table 5, a 4-marker autoantibody detection set of Table 6, a 5-marker autoantibody detection set of Table 7, a 6-marker autoantibody detection set of Table 8, or a 7-marker autoantibody detection set of Table 9, and correlating the immune reactivity of the test sample to the biomarker detection panel with a diagnosis, stage, or prognosis of prostate cancer. In one exemplary embodiment, a biomarker detection panel comprises a 3-marker detection set, including HEYL, RNF14 and PCNA. In one exemplary 4-biomarker embodiment, the biomarker detection panel comprises: an anti-IL-6 antibody, TRA1-SP, XLKD1, and PCNA. In one illustrative 5-biomarker embodiment, the biomarker detection panel comprises: SPRR1B, CCNA1, ERG, CCNB1, PSIP1. In one illustrative 6-biomarker embodiment, the biomarker detection panel comprises: ERBB2, CCNA1, KHDRBS1, RASSF1, NRP1, PCNA. In one illustrative 7-biomarker embodiment, the biomarker detection panel comprises: HEYL, BDKRB2, PSAP, MAD1L1, CCNA1, ERG, PCNA.

The biomarker detection panels can optionally include additional autoantibody capture molecules such as but not limited to autoantibody capture molecules from Table 11a, Table 1, Table 3, or Table 10.

An autoantibody capture molecule present in a biomarker detection panel can be a protein referred to as a target antigen (for example, a target antigen listed in Table 11a, Table 1, Table 3, or Table 10), including variants or modified forms thereof, or fragments thereof, that detect autoantibodies. An autoantibody capture molecule can also be an antibody that can bind an autoantigen that is complexed with one or more autoantibodies. A biomarker detection panel can comprise one or more target antigens and one or more target antibodies.

In addition to autoantibody capture molecules, a biomarker detection panel used to detect prostate cancer can optionally include one or more antibodies that capture antigens that are not necessarily complexed with autoantibodies. Binding of one or more test antigens of the biomarker detection panel to autoantibodies in the test sample and binding of one or more antibodies of the biomarker detection panel to antigens in the test sample can be detected and analyzed in determining the presence of prostate cancer in the individual, or distinguishing between the BPH and prostate cancer in a subject. An antibody present on a biomarker detection panel of the invention can be any antibody, including but not limited to an antibody that specifically binds any of the autoantigens of Table 1. Such antibodies can be used to detect antigens in a sample in the same assays used to detect autoantibodies using the autoantibody capture molecules. For example, in some embodiments of the methods directly or indirectly labeled secondary antibodies can be added to the panel prior to detection, in which the antibodies recognize the antigen captured by an antibody of the panel.

In certain embodiments, the pattern of immune reactivity is determined by quantitating the amount of binding of the one or more autoantibodies to target antigens. The quantitation can be absolute or relative. The quantitation can include normalizing the detection values with respect to one or more controls that are preferably but optionally provided in the same detection assay. In some embodiments, the method includes detecting a level of binding of antibodies of a sample to two or more autoantibody capture molecules, wherein the level of binding is above a threshold or cutoff value.

As will be understood, one or more test antigens may have poor diagnostic or prognostic value when considered alone, but when used as part of a panel that includes other reagents for biomarker detection (such as but not limited to other test antigens), such test antigens can contribute to making a particular diagnosis or prognosis. In preferred embodiments, particular threshold values for one or more test antigens in a biomarker detection panel are not relied upon to determine if a profile of marker levels obtained from a subject are indicative of a particular diagnosis or prognosis. Rather, the present invention may utilize an evaluation of the entire marker profile of a biomarker detection panel by plotting ROC curves for the sensitivity of a particular biomarker detection panel versus 1-(specificity) for the panel at various threshold cutoffs. The analyses performed herein determined threshold levels using logistic regression analysis, but similar results has been obtained using K-nearest neighbor analysis (statistical analyses are known in the art, and are described in detail in references such as, for example, Hastings, Tibshirani, and Friedman (2003) Elements of Statistical Learning, Springer). In these methods, a profile of biomarker measurements from a sample of an individual is considered together to provide an overall probability (expressed either as a numeric score or as a percentage risk) that an individual has prostate cancer, for example. In such embodiments, an increase in a certain subset of biomarkers (such as a subset of biomarkers that includes one or more autoantibodies) may be sufficient to indicate a particular diagnosis (or prognosis) in one patient, while an increase in a different subset of biomarkers (such as a subset of biomarkers that includes one or more autoantibodies) may be sufficient to indicate the same or a different diagnosis (or prognosis) in another patient. Weighting factors may also be applied to one or more biomarkers being detected. As one example, when a biomarker is of particularly high utility in identifying a particular diagnosis or prognosis, it may be weighted so that at a given level it alone is sufficient to indicate a positive diagnosis. In another example, a weighting factor may provide that no given level of a particular marker is sufficient to signal a positive result, but only signals a result when another marker also contributes to the analysis.

Increasing the specificity of a diagnostic test also decreases its sensitivity to some degree. In exemplary embodiments of the invention, the biomarker detection panel provides a test for the presence of prostate cancer in an individual that has at least as high a sensitivity as the currently used PSA test (that is greater than 80%), and a lower false positive rate than the currently used PSA test (about 75% false positives). The biomarker detection panel used in the methods of the invention in some exemplary embodiments has a specificity of 80% or greater, 85% or greater, 88% or greater, 90% or greater, 92% or greater, 94% or greater, 96% or greater, 98% or greater, or 100%, for diagnosing prostate cancer, or for discriminating prostate cancer from BPH in an individual. The biomarker detection panel in exemplary embodiments has a sensitivity of 80% or greater, 82% or greater, 84% or greater, 86% or greater, 88% or greater, 90% or greater, 92% or greater, 94% or greater, 96% or greater, 98% or greater, or 100%, for diagnosing prostate cancer, or for discriminating prostate cancer from BPH in an individual. The biomarker detection panel in exemplary embodiments has a Bayesian specificity of 78% or greater, 80% or greater, 85% or greater, 88% or greater, 90% or greater, or 92% or greater for diagnosing prostate cancer, or for discriminating prostate cancer from BPH in an individual. The biomarker detection panel in exemplary embodiments has a Bayesian sensitivity of 80% or greater, 82% or greater, 84% or greater, 85% or greater, 90% or greater, or 95% or greater for diagnosing prostate cancer, or for discriminating prostate cancer from BPH in an individual. The biomarker detection panel in exemplary embodiments has a Bayesian specificity of 78% or greater, 80% or greater, 85% or greater, 88% or greater, 90% or greater, or 92% or greater. The biomarker detection panel in exemplary embodiments has a Bayesian accuracy of 80% or greater, 81% or greater, 84% or greater, 85% or greater, 85% or greater, 87% or greater, 90% or greater, 93% or greater, or 96% or greater for diagnosing prostate cancer, or for discriminating prostate cancer from BPH in an individual.

Determining the immune reactivity of the sample can be performed by detection of binding of antibodies of the sample to the autoantibody capture molecules of the biomarker detection panel, and can be done in separate assays, in which each autoantibody capture molecule of the panel is contacted independently by the sample, or in a single assay, in which multiple autoantibody capture molecules are contacted with the sample in a single assay. In the latter case, different autoantibody capture molecules are preferably spatially separated, such as by binding to separate solid support surfaces or by binding of individual autoantibodies to specific locations on a single solid support, so that binding to individual autoantibody capture molecules can be independently assessed. Assays for detecting binding, including immunoassays, are described herein.

The test sample can be contacted with a autoantibody capture molecule provided in solution phase, or autoantibody capture molecules can be provided bound to a solid support. Detection of autoantibodies can be performed using an immunoassay, which can be in various formats as described in detail below. Detection of binding in certain illustrative embodiments makes use of one or more solid supports to which the autoantibody capture molecule is immobilized and to which the sample from an individual, in this case a human subject, is applied. The detection can be performed on any solid or semi-solid support, such as a gel, matrix, bead, particle, filament, fiber, rod, dish, plate, well, sheet, filter, strip, membrane, slide, chip, or array, such as a protein array, which can be a microarray, and can optionally be a high density microarray.

After incubation of the sample with the immobilized autoantibody capture molecules, or optionally, concurrently with the incubation of the sample, an antibody that is reactive against human antibodies (for example, an anti-human IgG antibody that is from a species other than human, for example, goat, rabbit, pig, mouse, etc.) is applied to the solid support with which the sample is incubated. The anti-human IgG antibody is directly or indirectly labeled. In some embodiments, the anti-human IgG antibody is labeled in one or more additional steps after the anti-human IgG antibody is contacted with the immobilized autoantibody capture molecule that has been contacted with a sample. After removing nonspecifically bound antibody, signal from the label that is significantly above background level is indicative of binding of a human antibody from the sample to an autoantibody capture molecule on the solid support.

The methods of diagnosing prostate cancer by contacting a sample from an individual with a biomarker detection panel can be repeated over time, for example, to monitor a pre-cancerous or pre-malignant state, or to monitor regression, progression, or recurrence of prostate cancer after or during a treatment or treatment regime (such as, for example, surgery, radiation therapy, chemotherapy, etc.). The results of a diagnostic test that determines the immune reactivity of a patient sample to a test antigen can be compared with the results of the same diagnostic test done at an earlier time. Significant differences in immune reactivity over time can contribute to a diagnosis or prognosis of prostate cancer.

In some embodiments, the individual has had at least one examination or diagnostic test that has indicated the presence of BPH or prostate cancer. In some embodiments, the individual is a male of age 50 or older. In some embodiments, the individual is a male with enlarged prostate. The individual can be a male previously diagnosed with BPH or PIN. The individual can have one or more risk factors for prostate cancer such as, for example, being age 55 or older, being African-American, or having a family history of prostate cancer. The individual can have one or more positive indicators of prostate cancer, such as, for example, a PSA level of greater than 4 ng per ml, a PSA level of greater than 10 ng per ml, a significant rise in PSA level over time, a free PSA to total PSA ratio of 0.25 or less, prostate abnormalities detected in a DRE, or a positive PCA3 urine test. The provided values are examples only, and test values used for diagnosis may differ. In some embodiments, for example, the individual can have PSA levels of less than 4 ng per ml.

In some embodiments, the method further includes testing for one or more additional indicators of prostate cancer, for example, PSA level or the PCA3 transcript level. The testing can be performed at the same time as determining the immune reactivity of the sample to a target antigen, or can be performed earlier or later than the test to detect autoantibodies to target antigens. Such additional indicators can contribute to the diagnosis.

A particular immune reactivity pattern can be correlated with a diagnosis of prostate cancer using statistical analysis based on comparison of immune reactivity of samples from prostate cancer patients and individuals not exhibiting prostate cancer using the same biomarker sets, as illustrated in the Examples below. Algorithms can be applied to the analysis of the binding patterns of test samples that can preferably be provided in computer readable format and integrated with signal detection devices to correlate binding patterns of samples with diagnoses of prostate cancer.

As will be understood, for any particular biomarker, a distribution of biomarker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a biomarker changes with the disease) the test is considered to be abnormal and below which the test is considered to be normal. Receiver Operating Characteristic curves, or “ROC” curves, are typically generated by plotting the value of a variable versus its relative frequency in “normal” and “disease” populations. The area under the ROC curve is a measure of the probability that the perceived measurement will allow correct identification of a condition. ROC curves can also be generated using relative, or ranked, results. Methods of generating ROC curves and their use are well known in the art. See, e.g., Hanley et al., Radiology 143: 29-36 (1982).

### Immunoassays

Immunoassays can be employed in any of the foregoing embodiments. Virtually any immunoassay technique known in the art can be used to detect antibodies that bind an antigen according to methods and kits of the present invention. Such immunoassay methods include, without limitation, radioimmunoassays, immunohistochemistry assays, competitive-binding assays, Western Blot analyses, ELISA assays, sandwich assays, test strip-based assays, assays using immunoprecipitation, assays combining antibody binding with two-dimensional gel electrophoresis (2D electrophoresis) and non-gel based approaches such as mass spectrometry or protein interaction profiling, all known to those of ordinary skill in the art. These methods may be carried out in an automated manner, as is known in the art. Such immunoassay methods may also be used to detect the binding of antibodies in a sample to a target antigen.

In one example of an ELISA method, the method includes incubating a sample with a target protein (such as an autoantibody capture molecule) and incubating the reaction product formed (which includes the captured autoantibody of the sample) with a binding partner, such as a secondary antibody (for example, an anti-species specific antibody), that binds to the reaction product by binding to an antibody from the sample that associated with the target protein to form the reaction product. In some cases these may comprise two separate steps, in others, the two steps may be simultaneous, or performed in the same incubation step. Examples of methods of detection of the binding of the target protein to an antibody include the use of an anti-human IgG (or other isotype specific) antibody or protein A. This detection antibody may be fluorescently labeled, or directly or indirectly linked to, for example, an alkaline phosphatase or a peroxidase, such as horseradish peroxidase. The CARD technique can optionally be employed to enhance the signal generated by a substrate converted by a peroxidase enzyme (Bobrow et al. (1989) J. Immunol. Methods 125: 279-285; Bhattacharya et al. (1999) J. Immunol. Methods 227: 31-39).

Using microarrays for immunoassays allows the simultaneous analysis of multiple proteins. For example, target antigens or antibodies that recognize biomarkers that may be present in a sample are immobilized on microarrays. Then, the biomarker antibodies or proteins, if present in the sample, are captured on the cognate spots on the array by incubation of the sample with the microarray under conditions favoring specific antigen-antibody interactions. The binding of protein or antibody in the sample can then be determined using secondary antibodies or other binding labels, proteins, or analytes. Comparison of proteins or antibodies found in two or more different samples can be performed using any means known in the art. For example, a first sample can be analyzed in one array and a second sample analyzed in a second array that is a replica of the first array.

The term “sandwich assay” refers to an immunoassay where the molecule to be detected is sandwiched between two binding reagents, which are typically antibodies. The first binding reagent/antibody is attached to a surface and the second binding reagent/antibody comprises a detectable moiety or label. In exemplary embodiments of the present invention, the first binding reagent is an autoantibody capture molecule, which can be an antigen or antibody, and the second binding reagent is anti-species specific antibody which can be directly or indirectly labeled, either when applied to the capture molecules to which sample has been added, or can be directly or indirectly labeled in a subsequent step. Examples of labels include, for example and without limitation: fluorophores, chromophores, enzymes that generate a detectable signal, or epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822 and 6,268,222), or quantum dot technology (for example, as described in U.S. Pat. No. 6,306,610).

A variety of different solid and semi-solid phase substrates can be used to detect a protein or antibody in a sample, or to quantitate or determine the concentration of a protein or antibody in a sample. The choice of substrate can be readily made by those of ordinary skill in the art, based on convenience, cost, skill, or other considerations. Useful substrates include without limitation: gels, matrices, beads, particles, bottles, surfaces, substrates, fibers, wires, framed structures, tubes, filaments, plates, sheets, filters, strips, and wells. These substrates can be made from: polystyrene, polypropylene, polycarbonate, glass, silica, silicon, plastic, metal, alloy, ceramics, cellulose, cellulose derivatives, nylon, coated surfaces, acrylamide or its derivatives and polymers thereof, agarose, or latex, or combinations thereof. This list is illustrative rather than exhaustive.

After contacting the sample with the biomarker detection panel, the panel is incubated under conditions of temperature, ionic strength, and pH compatible with antibody-antigen binding and for a time sufficient to allow antigen-antibody binding to occur. In preferred embodiments, after one or more washing steps, binding reagents (in exemplary embodiments species-specific antibodies) for detection are applied to the biomarker detection panel and also incubated with the biomarker detection panel under conditions of temperature, ionic strength, and pH compatible with binding and for a time sufficient to allow binding to occur.

Other methods of protein detection and measurement described in the art can be used as well. For example, a single antibody can be coupled to beads or to a well in a microwell plate, and quantitated by immunoassay. In this assay format, a single protein can be detected in each assay. The assays can be repeated with antibodies to many analytes to arrive at essentially the same results as can be achieved using the methods of this invention. Bead assays can be multiplexed by employing a plurality of beads, each of which is uniquely labeled in some manner. For example each type of bead can contain a pre-selected amount of a fluorophore. Types of beads can be distinguished by determining the amount of fluorescence (and/or wavelength) emitted by a bead. Such fluorescently labeled beads are commercially available from Luminex Corporation (Austin, Tex.; see the worldwide web address of luminexcorp.com). The Luminex assay is very similar to a typical sandwich ELISA assay, but utilizes Luminex microspheres conjugated to antibodies or proteins (Vignali, J. Immunol. Methods 243:243-255 (2000)).

The methodology and steps of various antibody assays are known to those of ordinary skill in the art. Additional information may be found, for example, in Antibodies: A Laboratory Manual, Cold Spring Harbor Laboratory, Chap. 14 (1988); Bolton and Hunter, “Radioimmunoassay and Related Methods,” in Handbook of Experimental Immunology (D. M. Weir, ed.), Blackwell Scientific Publications, 1996; and Current Protocols in Immunology, (John E. Coligan, et al., eds) (1993).

The antibodies used to perform the foregoing assays can include polyclonal antibodies, monoclonal antibodies and fragments thereof as described supra. Monoclonal antibodies can be prepared according to established methods (see, e.g., Kohler and Milstein (1975) Nature 256:495; and Harlow and Lane (1988) Antibodies: A Laboratory Manual (C.H.S.P., N.Y.)).

An antibody can be a complete immunoglobulin or an antibody fragment. Antibody fragments used herein, typically are those that retain their ability to bind an antigen. Antibodies subtypes include IgG, IgM, IgA, IgE, or an isotype thereof (e.g., IgG1, IgG2a, IgG2b or IgG3). Antibody preparations can by polyclonal or monoclonal, and can be chimeric, humanized or bispecific versions of such antibodies. Antibody fragments include but are not limited to Fab, Fab′, F(ab)′₂, Dab, Fv and single-chain Fv (ScFv) fragments. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. Antibodies sometimes are derivitized with a functional molecule, such as a detectable label (e.g., dye, fluorophore, radioisotope, light scattering agent (e.g., silver, gold)) or binding agent (e.g., biotin, streptavidin), for example.

Detection can use any means compatible with the label and format employed. For example, for a scanner can be used to detect the signal, such as a fluorescent signal, from arrays, filters, plate, or bead assays. A plate reader can also be used for ELISAs that use chromogenic reagents. Detection can also use scintillation counters or autoradiography/densitometry where the signal is generated by a radioisotope label.

Automated systems for performing immunoassays, such as those utilized in the methods herein, are widely known and used in medical diagnostics. For example, random-mode or batch analyzer immunoassay systems can be used, as are known in the art. These can utilize magnetic particles or non-magnetic particles or microparticles and can utilize a fluorescence or chemiluminescence readout, for example. As non-limiting examples, the automated system can be the Beckman ACCESS paramagnetic-particle, chemiluminescent immunoassay, the Bayer ACS:180 chemiluminescent immunoassay or the Abbott AxSYM microparticle enzyme immunoassay. Such automated systems can be designed to perform methods provided herein for an individual antigen or for multiple antigens without multiple user interventions.

### Biomarker Detection Panels

The invention also provides biomarker detection panels for diagnosing, prognosing, monitoring, or staging prostate cancer, in which the biomarker detection panels comprise two or more autoantibody capture molecules selected from Table 1 or Table 11a, in which at least 50% of the proteins of the test panel are autoantibody capture molecules of Table 1 or Table 11a. The set of autoantibody capture molecules in a biomarker detection panel are associated, either electronically, for example by linking the identities and/or information about the purchase or use of particular autoantibody capture molecules that are members of a biomarker detection panel, or preferably, physically.

For example, each biomarker of a biomarker detection panel can be provided in isolated form, in separate tubes or vials or bound to separate solid supports such as strips or beads that are sold and/or shipped together, for example as part of a kit. The autoantibody capture molecules of a biomarker panel can also be mixed together in the same solution. The autoantibody capture molecules of a biomarker panel can also be physically associated by being bound to one or more solid supports in the form of beads, one or more matrices (e.g., gels or resins), or one or more dishes, wells, plates, slides, sheets, membranes, strips, filters, fibers, chips, or arrays.

In certain embodiments, isolated autoantibody capture molecules are formed into a detection panel by attaching them to the same solid support. In some preferred embodiments, the proteins of the biomarker detection panel are provided on a protein array in which 50% or more of the proteins on the array are autoantibody capture molecules of the biomarker detection panel. A protein array that comprises a biomarker panel can in some exemplary embodiments be a high-density array.

The invention also provides biomarker detection panels for diagnosing, prognosing, monitoring, or staging prostate cancer, in which the biomarker detection panels comprise 2, 3, 4, 5, 6, 7, 8, 9, 10, 15, 20, 25, 30, 35, 40, 45, 50 or more target antigens selected from Table 1 or Table 11a, or in certain preferred embodiments, Table 3, in which at least 50%, 55%, 60%, 65%, 70%, or 75% of the proteins of the test panel are proteins of Table 1, Table 11a, or Table 3, respectively. In some preferred embodiments, the proteins of the biomarker detection panel are provided on one or more solid supports, in which at least 50%, 55%, 60%, 65%, 70%, 75%, 80%, 85%, 90%, 95% or 100% of the proteins on the one or more solid supports to which the proteins of the panel are bound are of Table 1, Table 11a, or Table 3. In some preferred embodiments, the proteins of the biomarker detection panel are provided on a protein array in which at least 55%, 60%, 65%, 70%, or 75%, 80%, 85%, 90%, 95% or 100% of the proteins on the array are autoantibody capture molecules of the biomarker detection panel.

The invention provides biomarker detection panels for diagnosing, prognosing, monitoring, or staging prostate cancer, in which the biomarker detection panels comprise KDR or PIM-1, a variant thereof, or a fragment thereof. The invention provides biomarker detection panels for diagnosing, prognosing, monitoring, or staging prostate cancer, in which the biomarker detection panels comprise KDR and PIM-1, variants thereof, or fragments thereof. The invention provides biomarker detection panels for diagnosing, prognosing, monitoring, or staging prostate cancer, in which the biomarker detection panels comprise 2, 3, 4, 5, 6, 7, 8, 9, 10, 15, 20, 25, 30, 35, 40, 45, 50 or more target antigens selected from Table 1, that comprise one or both of KDR or PIM-1, fragments thereof, or variants thereof. In some preferred embodiments, the proteins of the biomarker detection panel that include one or both of KDR or PIM-1 are provided on one or more solid supports, in which at least 50%, 55%, 60%, 65%, 70%, 75%, 80%, 85%, 90%, 95% or 100% of the proteins on the one or more solid supports to which the proteins of the panel are bound are of Table 1. In some preferred embodiments, the proteins of the biomarker detection panel are provided on a protein array in which at least 55%, 60%, 65%, 70%, or 75%, 80%, 85%, 90%, 95% or 100% of the proteins on the array are autoantibody capture molecules of the biomarker detection panel.

In some embodiments, the biomarker detection panel used in the methods of the invention includes 3, 4, 5, 6, 7, 8, 9, 10, 11, or 12 proteins of Table 1, Table 11a, or Table 3. In some embodiments, the biomarker detection panel used in the methods of the invention includes 13, 14, 15, 16, 17, 18, 19, 20, or more proteins of Table 1, Table 11a, or Table 3. In some embodiments, the test sample is contacted with a biomarker detection panel comprising 21, 22, 23, 24, 25, 26, 27, 28, 29, or 30 proteins of Table 1, Table 11a, or Table 3. A biomarker detection panel can comprise between 30 and 35 autoantibody capture molecules; between 35 and 40 autoantibody capture molecules; between 40 and 45 autoantibody capture molecules; between 45 and 50 autoantibody capture molecules; between 50 and 55 autoantibody capture molecules; between 55 and 60 autoantibody capture molecules; between 60 and 65 autoantibody capture molecules; between 65 and 70 autoantibody capture molecules; between 70 and 75 autoantibody capture molecules; between 75 and 80 autoantibody capture molecules; between 80 and 85 autoantibody capture molecules; between 85 and 90 autoantibody capture molecules; between 90 and 95 autoantibody capture molecules; between 95 and 100 autoantibody capture molecules, between 100 and 105 autoantibody capture molecules, or between 105 and 110 autoantibody capture molecules of Table 1, Table 11a, or Table 3.

One embodiment of the invention provides a biomarker panel comprising three autoantibody capture molecules from Table 1 or Table 11a. Table 5a provides ten autoantibody detection sets (labeled 5-1 through 5-10) having three autoantibody capture molecules. Table 5b shows the specificity and sensitivity of these biomarker panels, including their Bayesian accuracy. Both tables use a known positive control, such as mouse anti human IgG1, to normalize the data. In one embodiment, the biomarker detection panel comprising three autoantibody capture molecules is a panel provided in Table 5a.

TABLE 5a
Three Marker Biomarker Detection Panels, ranked by Bayesian
Accuracy, Identities of Markers

| Normalization Method | Panel | Marker 1 | Marker 2 | Marker 3 |
|---|---|---|---|---|
| Mouse Anti Human IgG1 | 5-1 | TOP2A | COVA1 | RASSF1 |
| Goat Anti-Human IgG | 5-2 | HEYL | RNF14 | PCNA |
| Mouse Anti Human | 5-3 | IMP-2 | XLKD1 | PCNA |
| Kappa 6.3 | | | | |
| Mouse Anti Human | 5-1 | TOP2A | COVA1 | RASSF1 |
| Kappa 6.3 | | | | |
| Protein L 1.6 | 5-4 | a-ACPP | RPS6KA1 | EIF4G1 |
| Mouse Anti Human | 5-5 | HEYL | CCNA1 | PCNA |
| Kappa 6.3 | | | | |
| Protein L 1.6 | 5-6 | a-II-6 | COVA1 | RASSF1 |
| Protein L 1.6 | 5-7 | SPRR1B | XLKD1 | CCNB1 |
| Mouse Anti Human | 5-8 | SPRR1B | CCNA1 | RASSF1 |
| Kappa 6.3 | | | | |
| Protein L 1.6 | 5-9 | a-II-6 | AZGP1(-SP) | COVA1 |
| Mouse Anti Human IgG1 | 5-10 | ZWINT | COVA1 | RASSF1 |

TABLE 5b
Three Marker Biomarker Detection Panels, ranked by Bayesian
Accuracy, Statistics

| Normalization | | | | | Bayesian | Bayesian | Bayesian |
|---|---|---|---|---|---|---|---|
| Method | Panel | AUC | Specificity | Sensitivity | Specificity | Sensitivity | Accuracy |
| Mouse Anti | 5-1 | 0.88158 | 91.67% | 89.47% | 85.71% | 85.71% | 87.88% |
| Human IgG1 | | | | | | | |
| Goat Anti- | 5-2 | 0.91228 | 83.33% | 89.47% | 78.57% | 85.71% | 84.85% |
| Human IgG | | | | | | | |
| Mouse Anti | 5-3 | 0.89474 | 83.33% | 89.47% | 78.57% | 85.71% | 84.85% |
| Human Kappa | | | | | | | |
| 6.3 | | | | | | | |
| Mouse Anti | 5-1 | 0.87719 | 83.33% | 89.47% | 78.57% | 85.71% | 84.85% |
| Human Kappa | | | | | | | |
| 6.3 | | | | | | | |
| Protein L 1.6 | 5-4 | 0.87281 | 83.33% | 89.47% | 78.57% | 85.71% | 84.85% |
| Mouse Anti | 5-5 | 0.86842 | 83.33% | 89.47% | 78.57% | 85.71% | 84.85% |
| Human Kappa | | | | | | | |
| 6.3 | | | | | | | |
| Protein L 1.6 | 5-6 | 0.86404 | 91.67% | 84.21% | 85.71% | 80.95% | 84.85% |
| Protein L 1.6 | 5-7 | 0.86842 | 83.33% | 84.21% | 78.57% | 80.95% | 81.82% |
| Mouse Anti | 5-8 | 0.85088 | 83.33% | 84.21% | 78.57% | 80.95% | 81.82% |
| Human Kappa | | | | | | | |
| 6.3 | | | | | | | |
| Protein L 1.6 | 5-9 | 0.81579 | 83.33% | 84.21% | 78.57% | 80.95% | 81.82% |
| Mouse Anti | 5-10 | 0.80702 | 83.33% | 84.21% | 78.57% | 80.95% | 81.82% |
| Human IgG1 | | | | | | | |

One embodiment of the invention provides a biomarker panel comprising four autoantibody capture molecules from Table 1 or Table 11a. Table 6a provides twenty-one autoantibody detection sets (labeled 6-1 through 6-21) having four autoantibody detection molecules. Table 6b shows the specificity and sensitivity of these biomarker panels, including their Bayesian accuracy. The tables use a known positive control, such as mouse anti human IgG1, to normalize the data. In one embodiment of the invention, the biomarker detection panel comprising four autoantibody detection molecules is a panel provided in Table 6a.

TABLE 6a
Four Marker Biomarker Detection Panels, ranked by Bayesian Accuracy,
Identities of Markers

| Panel | Normalization Method | Marker 1 | Marker 2 | Marker 3 | Marker 4 |
|---|---|---|---|---|---|
| 6-1 | Protein L 1.6 | a-II-6 | TRA1(-SP) | XLKD1 | PCNA |
| 6-2 | Mouse Anti Human Kappa | ZWINT | ACPP | CCNA1 | RASSF1 |
| | 6.3 | | | | |
| 6-3 | Mouse Anti Human Kappa | a-ACPP | CCNA1 | CD164 | RASSF1 |
| | 6.3 | | | | |
| 6-4 | Mouse Anti Human IgG1 | NCAM2 | KHDRBS1 | UBE2C | RASSF1 |
| 6-5 | Protein L 1.6 | a-II-6 | a-PSA (F) | RPS6KA1 | EIF4G1 |
| 6-6 | Mouse Anti Human IgG1 | SPRR1B | RASSF1 | XLKD1 | CCND1 |
| 6-7 | Mouse Anti Human IgG1 | TOP2A | RNF14 | CD164 | RASSF1 |
| 6-8 | Protein L 1.6 | a-II-8 | CCNA1 | CD164 | RASSF1 |
| 6-9 | Protein L 1.6 | a-II-6 | a-PSA(T) | RPS6KA1 | EIF4G1 |
| 6-10 | Goat Anti-Human IgG | PTGER3 | HMGA2 | EGFR | COVA1 |
| 6-11 | Mouse Anti Human IgG1 | PTGER3 | SPRR1B | NCAM2 | RASSF1 |
| 6-12 | Mouse Anti Human IgG1 | SPRR1B | STIP1 | RASSF1 | H3GLB1 |
| 6-13 | Protein L 1.6 | a-II-6 | RPS6KA1 | CD151 | EIF4G1 |
| 6-14 | Mouse Anti Human Kappa | a-II-6 | NRP1 | XLKD1 | PCNA |
| | 6.3 | | | | |
| 6-15 | Mouse Anti Human Kappa | a-II-8 | ACPP | CCNA1 | RASSF1 |
| | 6.3 | | | | |
| 6-16 | Human IgG 1.6 | PTGER3 | MAD1L1 | SPRR1B | CCNA1 |
| 6-17 | Protein L 1.6 | RCV1 | H3GLB1 | CCNB1 | PCNA |
| 6-18 | Goat Anti-Human IgG | MLH1 | RPS6KA1 | SPRR1B | RASSF1 |
| 6-19 | Protein L 1.6 | PTGER3 | PSMB4 | CCNA1 | COVA1 |
| 6-20 | Protein L 1.6 | ZWINT | H3GLB1 | CCNB1 | EIF4G1 |
| 6-21 | Protein L 1.6 | TRA1(-SP) | H3GLB1 | CCNB1 | EIF4G1 |

TABLE 6b
Four Marker Biomarker Detection Panels, ranked by Bayesian
Accuracy, Statistics

| | | | Sensi- | Bayesian | Bayesian | Bayesian |
|---|---|---|---|---|---|---|
| Panel | AUC | Specificity | tivity | Specificity | Sensitivity | Accuracy |
| 6-1 | 0.89035 | 91.67% | 94.74% | 85.71% | 90.48% | 90.91% |
| 6-2 | 0.86842 | 91.67% | 94.74% | 85.71% | 90.48% | 90.91% |
| 6-3 | 0.89474 | 83.33% | 94.74% | 78.57% | 90.48% | 87.88% |
| 6-4 | 0.85965 | 83.33% | 94.74% | 78.57% | 90.48% | 87.88% |
| 6-5 | 0.89474 | 83.33% | 89.47% | 78.57% | 85.71% | 84.85% |
| 6-6 | 0.89035 | 83.33% | 89.47% | 78.57% | 85.71% | 84.85% |
| 6-7 | 0.88596 | 91.67% | 84.21% | 85.71% | 80.95% | 84.85% |
| 6-8 | 0.88158 | 83.33% | 89.47% | 78.57% | 85.71% | 84.85% |
| 6-9 | 0.87281 | 83.33% | 89.47% | 78.57% | 85.71% | 84.85% |
| 6-10 | 0.86404 | 83.33% | 89.47% | 78.57% | 85.71% | 84.85% |
| 6-11 | 0.85965 | 83.33% | 89.47% | 78.57% | 85.71% | 84.85% |
| 6-12 | 0.85526 | 83.33% | 89.47% | 78.57% | 85.71% | 84.85% |
| 6-13 | 0.85088 | 91.67% | 84.21% | 85.71% | 80.95% | 84.85% |
| 6-14 | 0.8114 | 91.67% | 84.21% | 85.71% | 80.95% | 84.85% |
| 6-15 | 0.7807 | 83.33% | 89.47% | 78.57% | 85.71% | 84.85% |
| 6-16 | 0.88596 | 83.33% | 84.21% | 78.57% | 80.95% | 81.82% |
| 6-17 | 0.82018 | 83.33% | 84.21% | 78.57% | 80.95% | 81.82% |
| 6-18 | 0.81579 | 83.33% | 84.21% | 78.57% | 80.95% | 81.82% |
| 6-19 | 0.81579 | 83.33% | 84.21% | 78.57% | 80.95% | 81.82% |
| 6-20 | 0.80702 | 83.33% | 84.21% | 78.57% | 80.95% | 81.82% |
| 6-21 | 0.79386 | 83.33% | 84.21% | 78.57% | 80.95% | 81.82% |

One embodiment of the invention provides a biomarker panel comprising five autoantibody capture molecules from Table 1 or Table 11a. Table 7a provides seven autoantibody detection sets (labeled 7-1 through 7-7) having five autoantibody detection molecules. Table 7b shows the specificity and sensitivity of these biomarker panels, including their Bayesian accuracy. The tables use a known positive control, such as mouse anti human IgG1, to normalize the data. In one embodiment of the invention, the biomarker detection panel comprising five autoantibody detection molecules is a panel provided in Table 7a.

TABLE 7a
Five marker Biomarker Detection Panels, ranked by Bayesian Accuracy,
Identities of Markers

| Marker | Panel | AUC | Marker 1 | Marker 2 | Marker 3 | Marker 4 | Marker 5 |
|---|---|---|---|---|---|---|---|
| Protein L 1.6 | 7-1 | 0.91667 | SPRR1B | CCNA1 | ERG | CCNB1 | PSIP1 |
| Mouse Anti | 7-2 | 0.90351 | HEYL | CCNA1 | ERG | KHDRBS1 | PCNA |
| Human IgG1 | | | | | | | |
| Mouse Anti | 7-3 | 0.85965 | HEYL | ERBB2 | CCNA1 | KHDRBS1 | PCNA |
| Human IgG1 | | | | | | | |
| Human IgG 1.6 | 7-4 | 0.82018 | HEYL | RNF14 | CCNB1 | PCNA | EIF4G1 |
| Human IgG 1.6 | 7-5 | 0.74561 | HEYL | CCNA1 | MMP9 | BCLG | PCNA |
| Mouse Anti Biotin | 7-6 | 0.74561 | HEYL | BDKRB2 | RNF14 | HSPA5 | PCNA |
| 25 | | | | | | | |
| Human IgG 1.6 | 7-7 | 0.76316 | HEYL | ERBB2 | RNF14 | CCNB1 | PCNA |

TABLE 7b
Five marker Biomarker Detection Panels, ranked by Bayesian
Accuracy, Statistics

| | | Speci- | Sensi- | Bayesian | Bayesian | Bayesian |
|---|---|---|---|---|---|---|
| Panel | AUC | ficity | tivity | Specificity | Sensitivity | Accuracy |
| 7-1 | 0.91667 | 91.67% | 100.00% | 85.71% | 95.24% | 93.94% |
| 7-2 | 0.90351 | 83.33% | 100.00% | 78.57% | 95.24% | 90.91% |
| 7-3 | 0.85965 | 83.33% | 94.74% | 78.57% | 90.48% | 87.88% |
| 7-4 | 0.82018 | 91.67% | 89.47% | 85.71% | 85.71% | 87.88% |
| 7-5 | 0.74561 | 83.33% | 89.47% | 78.57% | 85.71% | 84.85% |
| 7-6 | 0.74561 | 83.33% | 89.47% | 78.57% | 85.71% | 84.85% |
| 7-7 | 0.76316 | 83.33% | 84.21% | 78.57% | 80.95% | 81.82% |

One embodiment of the invention provides a biomarker panel comprising six autoantibody capture molecules from Table 1 or Table 11a. Table 8a provides eighteen autoantibody detection sets (labeled 8-1 through 8-18) having six autoantibody detection molecules. Table 8b shows the specificity and sensitivity of these biomarker panels, including their Bayesian accuracy. The tables use a known positive control, such as mouse anti human IgG1, to normalize the data. In one embodiment of the invention, the biomarker detection panel comprising six autoantibody detection molecules is a panel provided in Table 8a.

TABLE 8a
Six Marker Biomarker Detection Sets, ranked by Bayesian Accuracy,
Identities of Markers

| Marker | | | | | | | |
|---|---|---|---|---|---|---|---|
| Set | Method | Marker 1 | Marker 2 | Marker 3 | Marker 4 | Marker 5 | Marker 6 |
| 8-1 | Mouse Anti Human Kappa | ERBB2 | CCNA1 | KHDRBS1 | RASSF1 | NRP1 | PCNA |
| | 6.3 | | | | | | |
| 8-2 | Mouse Anti Biotin 25 | MAD1L1 | SPRR1B | HMGA2 | ETS2 | IMP-2 | CCNB1 |
| 8-3 | Human IgG 1.6 | HEYL | BDKRB2 | PSAP | CCNA1 | ETS2 | PCNA |
| 8-4 | Human IgG 1.6 | HEYL | RPL30 | ERBB2 | CCNA1 | MMP9 | PCNA |
| 8-5 | Mouse Anti Human IgG1 | HEYL | CCNA1 | ERG | IMP-3 | KHDRBS1 | PCNA |
| 8-6 | Human IgG .4 | CCNA1 | RNF14 | ERG | MICB | CCNB1 | EIF4G1 |
| 8-7 | Mouse Anti Biotin 25 | HEYL | BDKRB2 | CCNA1 | ERG | RASSF1 | PCNA |
| 8-8 | Mouse Anti Biotin 25 | HEYL | RNF14 | ERG | AMACR | PCNA | EIF4G1 |
| 8-9 | Human IgG 1.6 | HEYL | PRL | BIRC5 | CCNA1 | CD164 | PCNA |
| 8-10 | Mouse Anti Biotin 25 | HSPB1 | SPRR1B | HMGA2 | ERG | IMP-2 | CCNB1 |
| 8-11 | Human IgG .4 | a-ACPP | PSAP | CCNA1 | ERG | MICB | EIF4G1 |
| 8-12 | Mouse Anti Human Kappa | a-II-8 | E7 | BDKRB2 | CCNA1 | RASSF1 | EIF4G1 |
| | 6.3 | | | | | | |
| 8-13 | Mouse Anti Biotin 25 | HEYL | RNF14 | STEAP | BCLG | CCNB1 | PCNA |
| 8-14 | Human IgG 1.6 | HEYL | CCNA1 | ERG | MMP9 | CCND1 | PCNA |
| 8-15 | Mouse Anti Biotin 25 | HEYL | BDKRB2 | CCNA1 | ETS2 | RASSF1 | PCNA |
| 8-16 | Human IgG 1.6 | HEYL | BDKRB2 | PTGER3 | ENO1 | CCNA1 | PCNA |
| 8-17 | Mouse Anti Biotin 25 | HEYL | CLDN3 | RNF14 | BCLG | CCNB1 | PCNA |
| 8-18 | Mouse Anti Biotin 25 | MYC | PSAP | NCAM2 | ETS2 | CCNB1 | EIF4G1 |

TABLE 8b
Six Marker Biomarker Detection Sets, ranked by Bayesian Accuracy,
Statistics

| | | | | Bayesian | Bayesian | Bayesian |
|---|---|---|---|---|---|---|
| Marker Set | AUC | Specificity | Sensitivity | Specificity | Sensitivity | Accuracy |
| 8-1 | 0.99123 | 100.00% | 94.74% | 92.86% | 90.48% | 93.94% |
| 8-2 | 0.91667 | 91.67% | 100.00% | 85.71% | 95.24% | 93.94% |
| 8-3 | 0.94298 | 100.00% | 89.47% | 92.86% | 85.71% | 90.91% |
| 8-4 | 0.9386 | 100.00% | 89.47% | 92.86% | 85.71% | 90.91% |
| 8-5 | 0.91228 | 83.33% | 100.00% | 78.57% | 95.24% | 90.91% |
| 8-6 | 0.83333 | 83.33% | 100.00% | 78.57% | 95.24% | 90.91% |
| 8-7 | 0.83333 | 83.33% | 100.00% | 78.57% | 95.24% | 90.91% |
| 8-8 | 0.89035 | 91.67% | 89.47% | 85.71% | 85.71% | 87.88% |
| 8-9 | 0.86404 | 91.67% | 89.47% | 85.71% | 85.71% | 87.88% |
| 8-10 | 0.84211 | 100.00% | 84.21% | 92.86% | 80.95% | 87.88% |
| 8-11 | 0.78947 | 83.33% | 94.74% | 78.57% | 90.48% | 87.88% |
| 8-12 | 0.78947 | 83.33% | 94.74% | 78.57% | 90.48% | 87.88% |
| 8-13 | 0.77193 | 91.67% | 84.21% | 85.71% | 80.95% | 84.85% |
| 8-14 | 0.74561 | 83.33% | 89.47% | 78.57% | 85.71% | 84.85% |
| 8-15 | 0.74561 | 83.33% | 89.47% | 78.57% | 85.71% | 84.85% |
| 8-16 | 0.70175 | 83.33% | 84.21% | 78.57% | 80.95% | 81.82% |
| 8-17 | 0.70175 | 83.33% | 84.21% | 78.57% | 80.95% | 81.82% |
| 8-18 | 0.70175 | 83.33% | 84.21% | 78.57% | 80.95% | 81.82% |

One embodiment of the invention provides a biomarker panel comprising seven autoantibody capture molecules from Table 1 or Table 11a. Table 9a provides eighty-one autoantibody detection sets (labeled 9-1 through 9-81) having seven autoantibody detection molecules. Table 9b shows the specificity and sensitivity of these biomarker panels, including their Bayesian accuracy. The tables use a known positive control, such as human IgG 1.6, to normalize the data. In one embodiment of the invention, the biomarker detection panel comprising seven autoantibody detection molecules is a panel provided in Table 9a.

TABLE 9a
Seven-marker Biomarker Detection Sets, ranked by Bayesian Accuracy,
Identities of Markers

| Panel | Method | Marker 1 | Marker 2 | Marker 3 | Marker 4 | Marker 5 | Marker 6 | Marker 7 |
|---|---|---|---|---|---|---|---|---|
| 9-1 | Human IgG 1.6 | HEYL | BDKRB2 | MAD1L1 | PSAP | CCNA1 | ERG | PCNA |
| 9-2 | Human IgG 1.6 | HEYL | BDKRB2 | PSAP | CCNA1 | ERG | IMP-3 | PCNA |
| 9-3 | Human IgG 1.6 | HEYL | BDKRB2 | PSAP | CCNA1 | ERG | PIM1 | PCNA |
| 9-4 | Human IgG 1.6 | HEYL | BDKRB2 | PSAP | CCNA1 | ETS2 | RDH11 | PCNA |
| 9-5 | Human IgG 1.6 | HEYL | BDKRB2 | PRSS8 | PSAP | CCNA1 | ERG | PCNA |
| 9-6 | Human IgG 1.6 | HEYL | BDKRB2 | PSAP | CCNA1 | ERG | KHDRBS1 | PCNA |
| 9-7 | Human IgG .4 | a-ACPP | MYC | PSAP | CCNA1 | ETS2 | MICB | EIF4G1 |
| 9-8 | Human IgG 1.6 | HEYL | BDKRB2 | HSPB1 | PSAP | CCNA1 | ERG | PCNA |
| 9-9 | Human IgG 1.6 | HEYL | BDKRB2 | PSAP | PSMB4 | CCNA1 | ERG | PCNA |
| 9-10 | Human IgG 1.6 | HEYL | ACPP | CCNA1 | ERG | TPD52 | PSA | PCNA |
| 9-11 | Human IgG 1.6 | a-PSA(T) | HEYL | BDKRB2 | PSAP | CCNA1 | ERG | PCNA |
| 9-12 | Human IgG 1.6 | HEYL | BDKRB2 | FLT1 | PSAP | CCNA1 | ERG | PCNA |
| 9-13 | Human IgG 1.6 | HEYL | BDKRB2 | MUC1 | PSAP | CCNA1 | ERG | PCNA |
| 9-14 | Human IgG 1.6 | HEYL | BDKRB2 | PSAP | SPRR1B | CCNA1 | ERG | PCNA |
| 9-15 | Human IgG 1.6 | HEYL | BDKRB2 | PSAP | CCNA1 | ERG | MMP9 | PCNA |
| 9-16 | Human IgG 1.6 | HEYL | BDKRB2 | PSAP | CCNA1 | ERG | TPD52 | PCNA |
| 9-17 | Human IgG 1.6 | HEYL | BDKRB2 | CCNA1 | ERG | MMP9 | CCKBR | PCNA |
| 9-18 | Human IgG 1.6 | HEYL | BDKRB2 | PSAP | CCNA1 | ETS2 | KHDRBS1 | PCNA |
| 9-19 | Human IgG .4 | a-ACPP | PSAP | QSCN6 | CCNA1 | ETS2 | MICB | EIF4G1 |
| 9-20 | Human IgG 1.6 | HEYL | BDKRB2 | HSPD1 | PSAP | CCNA1 | ERG | PCNA |
| 9-21 | Human IgG .4 | a-ACPP | BIRC5 | CCNA1 | RNF14 | MICB | CCNB1 | EIF4G1 |
| 9-22 | Human IgG .4 | a-ACPP | PSAP | CCNA1 | ETS2 | MICB | RDH11 | EIF4G1 |
| 9-23 | Human IgG .4 | BIRC5 | CCNA1 | RNF14 | MICB | ELAC1 | CCNB1 | EIF4G1 |
| 9-24 | Human IgG 1.6 | HEYL | BDKRB2 | CAV3 | PSAP | CCNA1 | ERG | PCNA |
| 9-25 | Human IgG 1.6 | HEYL | TP53 | BDKRB2 | PSAP | CCNA1 | ERG | PCNA |
| 9-26 | Human IgG 1.6 | HEYL | BDKRB2 | PSAP | CCNA1 | ERG | LGALS8 | PCNA |
| 9-27 | Human IgG 1.6 | HEYL | BDKRB2 | HMGA2 | CCNA1 | ERG | MMP9 | PCNA |
| 9-28 | Human IgG .4 | a-ACPP | PSAP | CCNA1 | ERG | MICB | UBE2C | EIF4G1 |
| 9-29 | Human IgG 1.6 | HEYL | BDKRB2 | PSAP | CCNA1 | ERG | ELAC1 | PCNA |
| 9-30 | Human IgG .4 | RCV1 | CCNA1 | RNF14 | ERG | MICB | CCNB1 | EIF4G1 |
| 9-31 | Human IgG .4 | BDKRB2 | CCNA1 | RNF14 | ERG | MICB | CCNB1 | EIF4G1 |
| 9-32 | Human IgG .4 | BDKRB2 | CCNA1 | RNF14 | ETS2 | MICB | CCNB1 | EIF4G1 |
| 9-33 | Human IgG 1.6 | HEYL | BDKRB2 | PSAP | CCNA1 | ERG | RASSF1 | PCNA |
| 9-34 | Human IgG 1.6 | HEYL | BDKRB2 | PSAP | CCNA1 | ERG | PSA | PCNA |
| 9-35 | Human IgG .4 | EIF3S3 | CCNA1 | RNF14 | ERG | MICB | CCNB1 | EIF4G1 |
| 9-36 | Human IgG 1.6 | HEYL | BDKRB2 | PSAP | RCV1 | CCNA1 | ERG | PCNA |
| 9-37 | Human IgG 1.6 | HEYL | BDKRB2 | CCNA1 | ERG | MMP9 | HOXB13 | PCNA |
| 9-38 | Human IgG 1.6 | HEYL | BDKRB2 | CCNA1 | MMP9 | ETS2 | HOXB13 | PCNA |
| 9-39 | Human IgG 1.6 | HEYL | ACPP | FLT1 | CCNA1 | ERG | MMP9 | PCNA |
| 9-40 | Human IgG 1.6 | HEYL | MET | BDKRB2 | CCNA1 | MMP9 | H3GLB1 | PCNA |
| 9-41 | Human IgG 1.6 | HEYL | BDKRB2 | BCL2 | PSAP | CCNA1 | ERG | PCNA |
| 9-42 | Human IgG 1.6 | HEYL | BDKRB2 | PSAP | CCNA1 | ERG | HOXB13 | PCNA |
| 9-43 | Human IgG 1.6 | HEYL | BDKRB2 | PSAP | CCNA1 | ERG | HIP1 | PCNA |
| 9-44 | Human IgG .4 | a-ACPP | PTGER3 | PSAP | CCNA1 | ETS2 | MICB | EIF4G1 |
| 9-45 | Human IgG 1.6 | a-BCL2 | HEYL | BDKRB2 | CCNA1 | ERG | MMP9 | PCNA |
| 9-46 | Human IgG 1.6 | a-II-6 | HEYL | BDKRB2 | PSAP | CCNA1 | ERG | PCNA |
| 9-47 | Human IgG 1.6 | HEYL | BDKRB2 | PTGS1 | PSAP | CCNA1 | ERG | PCNA |
| 9-48 | Human IgG 1.6 | HEYL | BDKRB2 | PSAP | CCNA1 | ERG | UBE2C | PCNA |
| 9-49 | Human IgG .4 | a-ACPP | PSAP | QSCN6 | CCNA1 | ERG | MICB | EIF4G1 |
| 9-50 | Human IgG .4 | MAD1L1 | CCNA1 | NRP1 | CCNB1 | CCKBR | PCNA | EIF4G1 |
| 9-51 | Human IgG 1.6 | HEYL | BDKRB2 | CXCR4 | PSAP | CCNA1 | ERG | PCNA |
| 9-52 | Human IgG 1.6 | HEYL | BDKRB2 | KDR | PSAP | CCNA1 | ERG | PCNA |
| 9-53 | Human IgG 1.6 | HEYL | BDKRB2 | PSAP | TRA1(- | CCNA1 | ERG | PCNA |
| | | | | | SP) | | | |
| 9-54 | Human IgG 1.6 | HEYL | BDKRB2 | PSAP | CCNA1 | ERG | ALOX15 | PCNA |
| 9-55 | Human IgG .4 | a-II-8 | CCNA1 | RNF14 | ERG | MICB | CCNB1 | EIF4G1 |
| 9-56 | Human IgG .4 | CCNA1 | RNF14 | ETS2 | MICB | PDLIM1 | CCNB1 | EIF4G1 |
| 9-57 | Human IgG 1.6 | HEYL | BDKRB2 | PSAP | CCNA1 | ERG | BCLG | PCNA |
| 9-58 | Human IgG 1.6 | HEYL | BDKRB2 | PSAP | CCNA1 | ERG | CCKBR | PCNA |
| 9-59 | Human IgG 1.6 | HEYL | ACPP | CCNA1 | ERG | MMP9 | KHDRBS1 | PCNA |
| 9-60 | Human IgG .4 | a-ACPP | PTEN | PSAP | CCNA1 | ERG | MICB | EIF4G1 |
| 9-61 | Human IgG .4 | a-ACPP | PSAP | CCNA1 | ERG | NCAM2 | MICB | EIF4G1 |
| 9-62 | Human IgG 1.6 | HEYL | BDKRB2 | BCL2 | CCNA1 | ERG | MMP9 | PCNA |
| 9-63 | Human IgG 1.6 | HEYL | BDKRB2 | PSAP | CUL4A | CCNA1 | ERG | PCNA |
| 9-64 | Human IgG 1.6 | HEYL | BDKRB2 | PSAP | CCNA1 | ERG | BRD2 | PCNA |
| 9-65 | Human IgG 1.6 | HEYL | BDKRB2 | PSAP | CCNA1 | ERG | CD164 | PCNA |
| 9-66 | Human IgG 1.6 | HEYL | BDKRB2 | CCNA1 | ERG | GDF15(- | HOXB13 | PCNA |
| | | | | | | SP) | | |
| 9-67 | Human IgG 1.6 | HEYL | MET | BDKRB2 | CCNA1 | MMP9 | TMPRSS2 | PCNA |
| 9-68 | Human IgG 1.6 | HEYL | BDKRB2 | BIRC5 | PSAP | CCNA1 | ERG | PCNA |
| 9-69 | Human IgG .4 | a-Pter2 | PTGER3 | HSPB1 | HSPD1 | CCNA1 | MICB | EIF4G1 |
| 9-70 | Human IgG .4 | a-ACPP | BIRC5 | PSAP | CCNA1 | GDF15(- | MICB | EIF4G1 |
| | | | | | | SP) | | |
| 9-71 | Human IgG 1.6 | HEYL | BDKRB2 | PSAP | CCNA1 | ERG | STEAP | PCNA |
| 9-72 | Human IgG .4 | a-ACPP | PTGER3 | PSAP | CCNA1 | ERG | MICB | EIF4G1 |
| 9-73 | Human IgG .4 | a-ACPP | PSAP | CCNA1 | ERG | MICB | RDH11 | EIF4G1 |
| 9-74 | Human IgG .4 | a-ACPP | PSAP | CCNA1 | GDF15(- | ETS2 | MICB | EIF4G1 |
| | | | | | SP) | | | |
| 9-75 | Human IgG .4 | PRL | HSPB1 | EIF3S3 | CCNA1 | ERG | MICB | EIF4G1 |
| 9-76 | Human IgG 1.6 | a-CXCR4 | HEYL | ACPP | CCNA1 | ERG | PSA | PCNA |
| 9-77 | Human IgG 1.6 | HEYL | BDKRB2 | PSAP | CCNA1 | ERG | EGFR | PCNA |
| 9-78 | Human IgG 1.6 | HEYL | BDKRB2 | RPL30 | PSAP | CCNA1 | ERG | PCNA |
| 9-79 | Human IgG .4 | CCNA1 | RNF14 | ERG | MICB | PDLIM1 | CCNB1 | EIF4G1 |
| 9-80 | Human IgG .4 | CCNA1 | RNF14 | ERG | MICB | MIB1 | CCNB1 | EIF4G1 |
| 9-81 | Human IgG 1.6 | HEYL | BDKRB2 | PSAP | SFRP4 | CCNA1 | ERG | PCNA |

TABLE 9b
Seven-marker Biomarker Detection Sets, ranked by Bayesian Accuracy,
Statistics

| | | | | Bayesian | Bayesian | Bayesian |
|---|---|---|---|---|---|---|
| Panel | AUC | Specificity | Sensitivity | Specificity | Sensitivity | Accuracy |
| 9-1 | 1 | 100.00% | 100.00% | 92.86% | 95.24% | 96.97% |
| 9-2 | 1 | 100.00% | 100.00% | 92.86% | 95.24% | 96.97% |
| 9-3 | 1 | 100.00% | 100.00% | 92.86% | 95.24% | 96.97% |
| 9-4 | 1 | 100.00% | 100.00% | 92.86% | 95.24% | 96.97% |
| 9-5 | 0.99561 | 91.67% | 100.00% | 85.71% | 95.24% | 93.94% |
| 9-6 | 0.99123 | 91.67% | 100.00% | 85.71% | 95.24% | 93.94% |
| 9-7 | 0.94737 | 100.00% | 94.74% | 92.86% | 90.48% | 93.94% |
| 9-8 | 0.94737 | 100.00% | 94.74% | 92.86% | 90.48% | 93.94% |
| 9-9 | 0.94737 | 100.00% | 94.74% | 92.86% | 90.48% | 93.94% |
| 9-10 | 0.94737 | 100.00% | 94.74% | 92.86% | 90.48% | 93.94% |
| 9-11 | 0.91667 | 91.67% | 100.00% | 85.71% | 95.24% | 93.94% |
| 9-12 | 0.91667 | 91.67% | 100.00% | 85.71% | 95.24% | 93.94% |
| 9-13 | 0.91667 | 91.67% | 100.00% | 85.71% | 95.24% | 93.94% |
| 9-14 | 0.91667 | 91.67% | 100.00% | 85.71% | 95.24% | 93.94% |
| 9-15 | 0.91667 | 91.67% | 100.00% | 85.71% | 95.24% | 93.94% |
| 9-16 | 0.91667 | 91.67% | 100.00% | 85.71% | 95.24% | 93.94% |
| 9-17 | 0.91667 | 91.67% | 100.00% | 85.71% | 95.24% | 93.94% |
| 9-18 | 0.98246 | 100.00% | 89.47% | 92.86% | 85.71% | 90.91% |
| 9-19 | 0.94298 | 100.00% | 89.47% | 92.86% | 85.71% | 90.91% |
| 9-20 | 0.94298 | 100.00% | 89.47% | 92.86% | 85.71% | 90.91% |
| 9-21 | 0.9386 | 91.67% | 94.74% | 85.71% | 90.48% | 90.91% |
| 9-22 | 0.9386 | 100.00% | 89.47% | 92.86% | 85.71% | 90.91% |
| 9-23 | 0.9386 | 91.67% | 94.74% | 85.71% | 90.48% | 90.91% |
| 9-24 | 0.9386 | 100.00% | 89.47% | 92.86% | 85.71% | 90.91% |
| 9-25 | 0.93421 | 100.00% | 89.47% | 92.86% | 85.71% | 90.91% |
| 9-26 | 0.91228 | 83.33% | 100.00% | 78.57% | 95.24% | 90.91% |
| 9-27 | 0.91228 | 83.33% | 100.00% | 78.57% | 95.24% | 90.91% |
| 9-28 | 0.90789 | 91.67% | 94.74% | 85.71% | 90.48% | 90.91% |
| 9-29 | 0.90789 | 91.67% | 94.74% | 85.71% | 90.48% | 90.91% |
| 9-30 | 0.89474 | 100.00% | 89.47% | 92.86% | 85.71% | 90.91% |
| 9-31 | 0.86842 | 91.67% | 94.74% | 85.71% | 90.48% | 90.91% |
| 9-32 | 0.86842 | 91.67% | 94.74% | 85.71% | 90.48% | 90.91% |
| 9-33 | 0.86842 | 91.67% | 94.74% | 85.71% | 90.48% | 90.91% |
| 9-34 | 0.86842 | 91.67% | 94.74% | 85.71% | 90.48% | 90.91% |
| 9-35 | 0.83333 | 83.33% | 100.00% | 78.57% | 95.24% | 90.91% |
| 9-36 | 0.83333 | 83.33% | 100.00% | 78.57% | 95.24% | 90.91% |
| 9-37 | 0.83333 | 83.33% | 100.00% | 78.57% | 95.24% | 90.91% |
| 9-38 | 0.83333 | 83.33% | 100.00% | 78.57% | 95.24% | 90.91% |
| 9-39 | 0.83333 | 83.33% | 100.00% | 78.57% | 95.24% | 90.91% |
| 9-40 | 0.92982 | 100.00% | 84.21% | 92.86% | 80.95% | 87.88% |
| 9-41 | 0.92544 | 100.00% | 84.21% | 92.86% | 80.95% | 87.88% |
| 9-42 | 0.92105 | 100.00% | 84.21% | 92.86% | 80.95% | 87.88% |
| 9-43 | 0.89912 | 91.67% | 89.47% | 85.71% | 85.71% | 87.88% |
| 9-44 | 0.86404 | 91.67% | 89.47% | 85.71% | 85.71% | 87.88% |
| 9-45 | 0.86404 | 91.67% | 89.47% | 85.71% | 85.71% | 87.88% |
| 9-46 | 0.86404 | 91.67% | 89.47% | 85.71% | 85.71% | 87.88% |
| 9-47 | 0.86404 | 91.67% | 89.47% | 85.71% | 85.71% | 87.88% |
| 9-48 | 0.86404 | 91.67% | 89.47% | 85.71% | 85.71% | 87.88% |
| 9-49 | 0.85965 | 91.67% | 89.47% | 85.71% | 85.71% | 87.88% |
| 9-50 | 0.85965 | 91.67% | 89.47% | 85.71% | 85.71% | 87.88% |
| 9-51 | 0.85965 | 91.67% | 89.47% | 85.71% | 85.71% | 87.88% |
| 9-52 | 0.85965 | 91.67% | 89.47% | 85.71% | 85.71% | 87.88% |
| 9-53 | 0.85965 | 91.67% | 89.47% | 85.71% | 85.71% | 87.88% |
| 9-54 | 0.82456 | 83.33% | 94.74% | 78.57% | 90.48% | 87.88% |
| 9-55 | 0.82018 | 91.67% | 89.47% | 85.71% | 85.71% | 87.88% |
| 9-56 | 0.82018 | 91.67% | 89.47% | 85.71% | 85.71% | 87.88% |
| 9-57 | 0.82018 | 91.67% | 89.47% | 85.71% | 85.71% | 87.88% |
| 9-58 | 0.82018 | 91.67% | 89.47% | 85.71% | 85.71% | 87.88% |
| 9-59 | 0.82018 | 91.67% | 89.47% | 85.71% | 85.71% | 87.88% |
| 9-60 | 0.78947 | 83.33% | 94.74% | 78.57% | 90.48% | 87.88% |
| 9-61 | 0.78947 | 83.33% | 94.74% | 78.57% | 90.48% | 87.88% |
| 9-62 | 0.78947 | 83.33% | 94.74% | 78.57% | 90.48% | 87.88% |
| 9-63 | 0.78947 | 83.33% | 94.74% | 78.57% | 90.48% | 87.88% |
| 9-64 | 0.78947 | 83.33% | 94.74% | 78.57% | 90.48% | 87.88% |
| 9-65 | 0.78947 | 83.33% | 94.74% | 78.57% | 90.48% | 87.88% |
| 9-66 | 0.78947 | 83.33% | 94.74% | 78.57% | 90.48% | 87.88% |
| 9-67 | 0.92105 | 83.33% | 89.47% | 78.57% | 85.71% | 84.85% |
| 9-68 | 0.85088 | 91.67% | 84.21% | 85.71% | 80.95% | 84.85% |
| 9-69 | 0.81579 | 83.33% | 89.47% | 78.57% | 85.71% | 84.85% |
| 9-70 | 0.80702 | 91.67% | 84.21% | 85.71% | 80.95% | 84.85% |
| 9-71 | 0.80702 | 91.67% | 84.21% | 85.71% | 80.95% | 84.85% |
| 9-72 | 0.78509 | 83.33% | 89.47% | 78.57% | 85.71% | 84.85% |
| 9-73 | 0.78509 | 83.33% | 89.47% | 78.57% | 85.71% | 84.85% |
| 9-74 | 0.78509 | 83.33% | 89.47% | 78.57% | 85.71% | 84.85% |
| 9-75 | 0.78509 | 83.33% | 89.47% | 78.57% | 85.71% | 84.85% |
| 9-76 | 0.78509 | 83.33% | 89.47% | 78.57% | 85.71% | 84.85% |
| 9-77 | 0.78509 | 83.33% | 89.47% | 78.57% | 85.71% | 84.85% |
| 9-78 | 0.77632 | 83.33% | 89.47% | 78.57% | 85.71% | 84.85% |
| 9-79 | 0.77193 | 91.67% | 84.21% | 85.71% | 80.95% | 84.85% |
| 9-80 | 0.74561 | 83.33% | 89.47% | 78.57% | 85.71% | 84.85% |
| 9-81 | 0.74561 | 83.33% | 89.47% | 78.57% | 85.71% | 84.85% |

Individual markers (autoantibody capture molecules) that were present in at least 10% of two or more of the statistically significant classifiers of Tables 5, 6, 7, 8, and 9 are provided in Table 10. The number of normalization techniques (out of seven) in which the marker (autoantibody capture molecule) was present in 10% or more of the identified statistically significant classifiers is provided. Normalization was done with various positive controls at various concentrations. Additionally, the percentage of normalization techniques in which the (autoantibody capture molecule) was present in 10% or more of the identified statistically significant classifiers is provided.

TABLE 10
Target Antigens Having 10% or Above of Statistical Significant
Classifiers for Prostate Cancer versus BPH

| | Number of | |
|---|---|---|
| | Normalization | |
| | Techniques Marker | Percentage of |
| | was in 10% or | Normalization |
| Markers | Above of Classifiers | Techniques |
| CCNA1 | 6 | 85.71% |
| PCNA | 6 | 85.71% |
| HEYL | 5 | 71.43% |
| SPRR1B | 5 | 71.43% |
| RASSF1 | 5 | 71.43% |
| RNF14 | 4 | 57.14% |
| ERG | 4 | 57.14% |
| COVA1 | 4 | 57.14% |
| EIF4G1 | 4 | 57.14% |
| BDKRB2 | 3 | 42.86% |
| PTGER3 | 3 | 42.86% |
| PSAP | 3 | 42.86% |
| XLKD1 | 3 | 42.86% |
| CCNB1 | 3 | 42.86% |
| a-ACPP | 2 | 28.57% |
| a-II-6 | 2 | 28.57% |
| ZWINT | 2 | 28.57% |
| ERBB2 | 2 | 28.57% |
| TOP2A | 2 | 28.57% |
| RPS6KA1 | 2 | 28.57% |
| HMGA2 | 2 | 28.57% |
| NCAM2 | 2 | 28.57% |
| ETS2 | 2 | 28.57% |
| CD164 | 2 | 28.57% |
| IMP-2 | 2 | 28.57% |
| KHDRBS1 | 2 | 28.57% |
| H3GLB1 | 2 | 28.57% |

Biomarker detection panels of the invention specifically include but are not limited to any biomarker detection panels disclosed in this application. In some preferred embodiments, the biomarker panel includes at least one autoantibody capture molecule of Table 2. In some preferred embodiments, at least one of the proteins of the biomarker panel is selected from Table 10. In some preferred embodiments, a biomarker panel includes at least one biomarker detection set of Table 5, Table 6, Table 7, Table 8, or Table 9.

Biomarker detection panels of the invention can include proteins or protein fragments that are not antibodies as well as proteins that are antibodies, such as but not limited to antibodies to ACPP, BCL2, CXR4, IL-6, IL-8, PSA(free), PSA(total), or PTGER2. In some embodiments, a biomarker detection panel can includes an antibody to any of the target antigens of Table 1 or Table 11a. A biomarker detection panel of the invention can also include one or more antibodies that are not listed in Table 1 and are not antibodies to target antigens of Table 1 or Table 11a. As nonlimiting examples, antibodies to PAP or PSMA can also be part of a biomarker detection panel of the invention.

Also included in the invention is a composition that comprises a biomarker detection panel for diagnosing, prognosing, monitoring, or staging prostate cancer that comprises two or more autoantibody capture molecules selected from Table 1 or Table 11a, in which at least one of the two or more autoantibody capture molecules is bound to an autoantibody from a sample of an individual. The invention includes a biomarker detection panel for diagnosing, prognosing, monitoring, or staging prostate cancer that comprises 3, 4, 5, 6, 7, 8, 9, 10, 11, 12, 13, 14, 15, 16, 17, 18, 19, 20, 21, 22, 23, 24, 25, 26, 27, 28, 29, 30 or more autoantibody capture molecules selected from Table 1 or Table 11a, in which at least one of the two or more autoantibody capture molecules is bound to an autoantibody from a sample of an individual.

The invention provides biomarker detection panels for diagnosing, prognosing, monitoring, or staging prostate cancer, in which the biomarker detection panels comprise KDR and PIM-1, variants thereof, or fragments thereof, in which at least one of the two or more autoantibody capture molecules is bound to an autoantibody from a sample of an individual. The invention includes a biomarker detection panel for diagnosing, prognosing, monitoring, or staging prostate cancer that comprises 3, 4, 5, 6, 7, 8, 9, 10, 11, 12, 13, 14, 15, 16, 17, 18, 19, 20, 21, 22, 23, 24, 25, 26, 27, 28, 29, 30 or more autoantibody capture molecules, in which at least one of the autoantibody capture molecules is KDR or PIM-1, and in which at least one of the two or more autoantibody capture molecules is bound to an autoantibody from a sample of an individual. In some embodiments, at least one of the KDR or PIM-1 autoantibody capture molecules is bound to an autoantibody from a sample of an individual. In some embodiments, both of the KDR or PIM-1 autoantibody capture molecules are bound to an autoantibody from a sample of an individual.

Also included in the invention is a composition that comprises a biomarker detection panel for diagnosing, prognosing, monitoring, or staging prostate cancer that comprises two or more autoantibody capture molecules selected from Table 3, in which at least one of the autoantibody capture molecules of Table 3 of the array is bound to an autoantibody from a sample of an individual. Also included in the invention is a composition that comprises a biomarker detection panel for diagnosing, prognosing, monitoring, or staging prostate cancer that comprises two or more autoantibody capture molecules selected from Table 3, in which the array includes at least one autoantibody capture molecule of Table 10, and at least one of the autoantibody capture molecules of Table 3 of the array is bound to an autoantibody from a sample of an individual. The arrays having bound antibody from a sample can be arrays in which at least 55%, 60%, 65%, 70%, 75%, 80%, 85%, 90%, of 95% of the protein bound to the arrays are proteins of Table 3.

### Methods for Identifying Autoantigen/Autoantibody Biomarker Detection Panels

The present disclosure identifies a population of autoantibodies and panels thereof, associated with prostate cancer based on knowledge of biological pathways altered by cancer progression (including immunological pathways), analogy with other cancers, and literature searching to compile a list of proteins that were overexpressed, inappropriately expressed, or differentially modified or degraded in prostate cancer cells when compared with normal prostate cells. A similar method can be used with other cancers, such as, for example, breast cancer, lung cancer, colorectal cancer, brain cancer, stomach cancer, bladder cancer, pancreatic cancer, ovarian cancer, liver cancer, leukemia, etc. Accordingly, provided herein is a method for identifying one or more autoantigens or one or more panels of autoantigens that are differentially present in a sample from an individual having a target cancer from an individual not having the target cancer, that includes identifying proteins that are overexpressed, inappropriately expressed, or differentially modified or degraded in a target cancer compared with normal cells or cells of a benign condition, performing an immunoassay that compares the pattern of immune reactivity of a sample from an individual having the target cancer to a sample for an individual that does not have the cancer, against a biomarker detection panel that includes one or more, two or more, three or more, four or more five or more, 10 or more, 20 or more 25 or more, 100 or more, 200 or more 250 or more 500 or more, or 1000 or more of the identified proteins, wherein antibodies against the identified proteins that are differentially present in the sample from the individual having the target cancer compared to the sample from the individual not having the target cancer, identifies the identified protein as an autoantigen for the target cancer. In certain aspects, the method further comprises repeating the immunoassay for a group of samples from different individuals having the target cancer and repeating the immunoassay for a group of samples from different individuals not having the target cancer.

### Method for Synthesizing Protein Antigens

The methods, kits, and systems provided herein include autoantigens, which typically are protein antigens. To obtain protein antigens to be used in the methods provided herein, known methods can be used for making and isolating viral, prokaryotic or eukaryotic proteins in a readily scalable format, amenable to high-throughput analysis. For example, methods include synthesizing and purifying proteins in an array format compatible with automation technologies.

In some exemplary embodiments, proteins are expressed from gene constructs using in vitro synthesis systems or cell culture systems. Any expression construct having an inducible promoter to drive protein synthesis can be used in accordance with the methods of the invention. The expression construct may be, for example, tailored to the cell type to be used for transformation. Compatibility between expression constructs and host cells are known in the art, and use of variants thereof are also encompassed by the invention.

Therefore, in one embodiment, protein microarrays for the invention a method for making and isolating eukaryotic proteins comprising the steps of growing a cell transformed with a vector having a heterologous sequence operatively linked to a regulatory sequence, contacting the regulatory sequence with an inducer that enhances expression of a protein encoded by the heterologous sequence, lysing the cell, contacting the protein with a binding agent such that a complex between the protein and binding agent is formed, isolating the complex from cellular debris, and isolating the protein from the complex, wherein each step is conducted in a 96-well format. For example, bacterial, yeast, mammalian, or insect cells can be used for the production of proteins.

In a particular embodiment, eukaryotic proteins can be made and purified in a 96-array format (i.e., each site on the solid support where processing occurs is one of 96 sites), e.g., in a 96-well microtiter plate. In another embodiment, the solid support does not bind proteins (e.g., a non-protein-binding microtiter plate).

In certain embodiments, proteins are synthesized by in vitro translation according to methods commonly known in the art. For example, a wheat germ expression (WGE) system can be used to synthesize proteins used as autoantibody capture molecules. A variety of commercial WGE systems are available, the majority of this work has been performed using Cell Free Sciences WGE system (Yokahama Japan). Alternatively, proteins used as autoantibody capture molecules can be synthesized in other in vitro synthesis systems or in cell culture. As nonlimiting examples, E. coli in vitro translation systems or reticulocyte lysate in vitro translation systems can be used for synthesis of autoantibody capture molecules. Proteins used as autoantibody capture molecules can also be isolated from organisms, for example, from sera.

In some exemplary embodiments, proteins are synthesized in vitro or in culture systems as GST-fusion constructs, and are purified from cell culture or a cell-free expression system using GST-beads or columns. Invitrogen's Ultimate™ ORF clone collection is an ideal platform to generate a large number of antigens in a facile manner.

In a particular embodiment, the fusion proteins have GST tags and are affinity purified by contacting the proteins with glutathione beads. In further embodiment, the glutathione beads, with fusion proteins attached, can be washed in a 96-well box without using a filter plate to ease handling of the samples and prevent cross contamination of the samples.

In addition, fusion proteins can be eluted from the binding compound (e.g., glutathione bead) with elution buffer to provide a desired protein concentration. In a specific embodiment, fusion proteins are eluted from the glutathione beads with elution buffer to provide a desired protein concentration.

For purified proteins that will eventually be spotted onto microscope slides, the glutathione beads are separated from the purified proteins. In one example, all of the glutathione beads are removed to avoid blocking of the microarrays pins used to spot the purified proteins onto a solid support. In one embodiment, the glutathione beads are separated from the purified proteins using a filter plate, for example, comprising a non-protein-binding solid support. Filtration of the eluate containing the purified proteins should result in greater than 90% recovery of the proteins.

The elution buffer may, for example, comprise a liquid of high viscosity such as, for example, 15% to 50% glycerol, for example, about 40% glycerol. The glycerol solution stabilizes the proteins in solution, and prevents dehydration of the protein solution during the printing step using a microarrayer.

Purified proteins may, for example, be stored in a medium that stabilizes the proteins and prevents desiccation of the sample. For example, purified proteins can be stored in a liquid of high viscosity such as, for example, 15% to 50% glycerol, for example, in about 40% glycerol. In one example, samples may be aliquoted containing the purified proteins, so as to avoid loss of protein activity caused by freeze/thaw cycles.

The skilled artisan can appreciate that the purification protocol can be adjusted to control the level of protein purity desired. In some instances, isolation of molecules that associate with the protein of interest is desired. For example, dimers, trimers, or higher order homotypic or heterotypic complexes comprising an overproduced protein of interest can be isolated using the purification methods provided herein, or modifications thereof. Furthermore, associated molecules can be individually isolated and identified using methods known in the art (e.g., mass spectroscopy).

The protein antigens once produced, can be used in the biomarker panels, methods and kits provided herein as part of a “positionally addressable” array. The array includes a plurality of target antigens, with each target antigen being at a different position on a solid support. The array can include, for example, 1, 2, 3, 4, 5, 8, 9, 10, 11, 12, 13, 14, 15, 16, 17, 18, 19, 20, 21, 22, 23,24, 25, 26, 27, 28, 29, 30, 40, 50, 100, 200, 300, 400, or 500 different proteins. The array can include 1, 2, 3, 4, 5, 10, 15, 20, 25, 50, 100 or all the proteins of Table 1. In one aspect, the majority of proteins on an array includes proteins identified as autoantigens that can have diagnostic value for a particular disease or medical condition when provided together autoantigen biomarker detection panel.

In one aspect, the protein array is a bead-based array. In another aspect, the protein array is a planar array. Methods for making protein arrays, such as by contact printing, are well known. In some embodiments, the detection is performed on a protein array, which can be a microarray, and can optionally be a microarray that includes proteins at a concentration of at least 100/cm² or 1000/cm², or greater than 400/cm².

In this embodiment, amino-terminal tagged GST proteins were utilized. Proteins were placed into 386-well printing “masterplates”.

### Kits

In certain embodiments of the invention, kits are provided. Thus, in some embodiments, a kit is provided that comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30-34, 35-39, 40-44, 45-49, 50-54, 55-59, 60-64, 65-69, 70-74, 75-79, 80-84, 85-89, 90-94, 95-100, 100-105, or 106-108 of the autoantibody capture molecules provided in Table 1. A kit of the invention can include any of the biomarker detection panels disclosed herein, including, but not limited to, a biomarker panel comprising two or more autoantibody capture molecules of Table 1, a biomarker panel comprising two or more biomarker capture molecules of Table 1, in which one or more of the capture molecules is a protein of Table 3, a biomarker panel comprising two or more biomarker capture molecules of Table 3, a biomarker panel comprising two or more autoantibody capture molecules of Table 1, in which one or more of the capture molecules is an autoantibody capture molecule of Table 10, and a biomarker panel comprising a 3-marker biomarker detection set of Table 5, a 4-marker biomarker detection set of Table 6, a 5-marker biomarker detection set of Table 7, a 6-marker biomarker detection set of Table 8, or a 7-marker biomarker detection set of Table 9.

In some preferred embodiments, a kit of the invention can include an autoantibody capture molecule that binds an autoantibody to KDR (for example, KDR, or a variant or fragment thereof) or an autoantibody capture molecule that binds an autoantibody to PIM-1 (for example, PIM-1, or an antibody or fragment thereof). A kit of the invention can include an autoantibody capture molecule that binds an autoantibody to KDR (for example, KDR, or a variant or fragment thereof) and an autoantibody capture molecule that binds an autoantibody to PIM-1 (for example, PIM-1, or an antibody or fragment thereof). The kit can further include other autoantibody capture molecules, such as but not limited to those provided in Table 1. A kit can include biomarker detection panel that includes KDR, PIM-1, or both KDR and PIM-1. The detection panel can include any number of autoantibody capture molecules, for example, from one to 10, from 10-20, from 20-30, from 30-40, from 40-50, from 50-100, or more than 100.

A kit can include one or more positive controls, one or more negative controls, and/or one or more normalization controls.

The proteins of the kit may, for example, be immobilized on a solid support or surface. The proteins may, for example, be immobilized in an array. The protein microarray may use bead technology, such as the Luminex technology (Luminex Corp., Austin, Tex.). The test protein array may or may not be a high-density protein microarray that includes at least 100 proteins/cm². The kit can provide a biomarker detection panel of proteins as described herein immobilized on an array. At least 50%, at least 55%, at least 60%, at least 65%, at least 70%, at least 75%, at least 80%, at least 85%, at least 90%, or at least 95% of the proteins immobilized on the array can be proteins of the biomarker test pane. The array can include immobilized on the array one or more positive control proteins, one or more negative controls, and/or one or more normalization controls.

A kit may further comprise detection reagents and/or one or more reporter reagents to detect binding of human antibody to the proteins of the biomarker detection panel, such as, for example, an a species-specific antibody that binds to human antibodies, such as, for example, anti-human IgG antibody. The species specific antibody can be linked to a detectable label.

A kit may further comprise reagents useful for various immune reactivity assays, such as ELISA, or other immunoassay techniques known to those of skill in the art. The assays in which the kit reagents can be used may be competitive assays, sandwich assays, test strip assays, and the label may be selected from the group of well-known labels used for radioimmunoassay, fluorescent or chemiluminescence immunoassay.

A kit can include reagents described herein in any combination. For example, in one aspect, the kit includes a biomarker detection panel as provided herein immobilized on a solid support and anti-human antibodies for detection in solution or for detection on a solid support. The detection antibodies can comprise labels.

The kit can also include a program in computer readable form to analyze results of methods performed using the kits to practice the methods provided herein.

The kits of the present invention may also comprise one or more of the components in any number of separate containers, packets, tubes, vials, microtiter plates and the like, or the components may be combined in various combinations in such containers.

The kits of the present invention may also comprise instructions for performing one or more methods described herein and/or a description of one or more compositions or reagents described herein. Instructions and/or descriptions may be in printed form and may be included in a kit insert. A kit also may include a written description of an Internet location that provides such instructions or descriptions.

### EXAMPLES

The examples set forth below illustrate but do not limit the invention.

### Example 1

### Test Protein Array for Diagnostic Autoantigens

Biomarker detection panels were sought having at least as high a sensitivity as the standard PSA test (80%), with higher specificity.

### Experimental Design

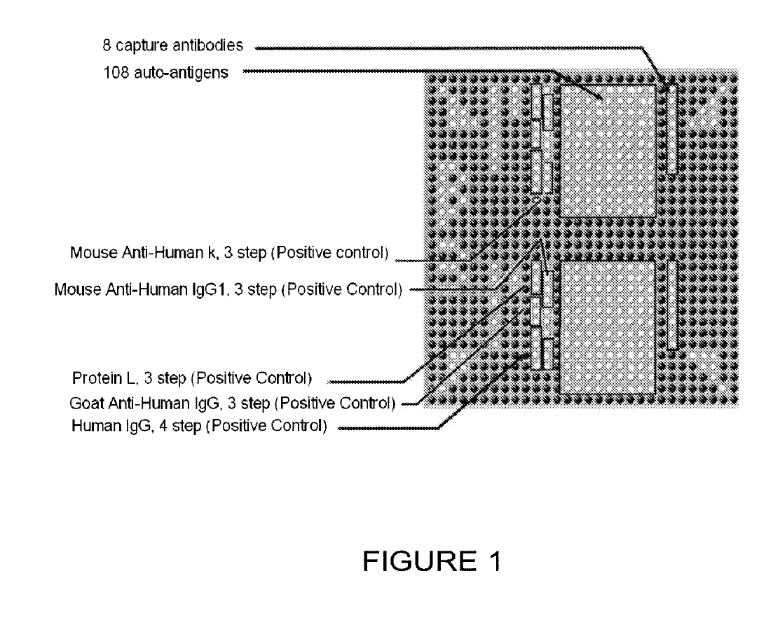

A protein array was fabricated by spotting proteins on a planar nitrocellulose substrate. The overall design of the array is depicted in FIG. 1, which shows half of an array used to test for the diagnostic utility of the 108 antigens and 8 antibodies listed in Table 1. The antigens and antibodies were selected based on biological experiments, knowledge of biological pathways altered by cancer progression (including immunological pathways), analogy with other cancers, and literature searching to compile a list of proteins that were overexpressed, inappropriately expressed, or differentially modified or degraded in prostate cancer cells when compared with normal prostate cells.

Table 1 provides in the first column (“Marker”) the term used throughout this application for the autoantibody capture molecule. The second column provides the term for the protein used by the Human Genome Organization (HUGO). Other names used for the protein in the scientific literature are provided in column 3, and column 4 provides the Genbank gene identifier and reference sequence ID. The table also provides in column 5 the method of synthesis and/or source of the protein.

The proteins were synthesized in vitro (103 out of the 108 using wheat germ extract, in most cases using Cell Free Sciences WGE system (Yokahama Japan)), or, in some cases, were obtained commercially as proteins synthesized in E. coli or isolated from human serum. A variety of clones available from Invitrogen Corp. (Carlsbad, Calif., Invitrogen.com), including the extensive human ORF collection, were used as templates. The cell-free expressed proteins were purified using purification tags (e.g., GST-fusion vectors using GST columns) and then quality-control verified (for correct molecular weight and purity) using high throughput electrophoresis (e.g., Agilent bioanalysers). Antibodies were obtained commercially.

The chip array (as shown in FIG. 1) also included as controls: 1137 empty spots (no protein), 35 Alexa 488 fiducials, 16 Alexa 555 fiducials, 24 Alexa 647 fiducials, glutathione S-transferase (GST) at 8 concentrations, bovine serum albumin (BSA) at 8 concentrations, mouse anti-human Kappa antibody at three concentrations (a positive control), mouse anti-human IgG1 antibody at three concentrations (a positive control), goat anti-mouse biotin at three concentrations, mouse anti-biotin at three concentrations, Protein L (an immunoglobulin binding protein from the bacterium Peptostreptococcus magnus) at three concentrations, goat anti-human IgG at three concentrations, and human IgG at three concentrations. Positive controls were spotted at from 5 to 8 different concentrations.

The qc-validated protein antigens were then printed onto slides with various surfaces, including but not limited to nitrocellulose, amine group or epoxy group modified surfaces. Nitrocellulose has the largest binding capacity of these surfaces, so it was used for the final test chip Ver.3. The antigens were printed in a manner so that each is represented using multiple, independent spots, located in geographically distinct spots multiple millimeters away from each other (FIG. 1). Multiple positive and negative control proteins that were specifically selected and designed for autoantigen profiling experiments, were spotted on the test chip Ver.3 such that they were interspersed around and within the antigen spots. Examples of positive and negative control spots are provided in FIG. 1 (fluorophore fiducials, GST protein, mouse anti-human antibodies, protein L, mouse anti-biotin, BSA protein, and human IgG) along with the tested antigens and antibodies.

The example chip design format shown in FIG. 1 comprises 8 capture antibodies, 108 auto-antigens, mouse anti-human K 3 step (positive control), mouse anti-human IgG1 3 step (positive control), goat anti-mouse biotin 3 step (biotinylation assay control), mouse anti-biotin 3 step (biotinylation assay control), protein L 3 step (positive control), human IgG 4 step (positive control), and 1137 empty spots. The array is printed in duplicate on each chip, so every spot on the chip is replicated a total of four times. The chip also utilizes a print buffer, BSA 2 step gradient (negative control), GST 8 step gradient (on-chip concentration quantification), 35 Alexa 488 Fiducials, 16 Alexa 555 Fiducials, 24 Alexa 647 Fiducials.

Protein concentrations for printing were approximately 150 micrograms per mL (ranging from 30 to 250 ug/mL). Approximately 10 uL protein aliquots in 50 mM Tris, 10 mM glutathione pH 8 were diluted 1:1 with this same buffer or Whatman printing buffer (Protein Arraying Buffer, 2× concentration, product number 10 485 331). Master plates were maintained at either 15° C. or at ˜4° C. during the printing process. Proteins were printed onto Whatman nitrocellulose slides (FAST slides, 1 pad, 11 um thick nitrocellulose, product number 10 484 182). Approximately 250 pL of protein solution was spotted, or multiples of between three to six times 250 pL spotted sequentially using a Scienion non-contact piezo-based printer (sciFLEXARRAYER S5 Piezo Dispenser). After spotting, slides were placed into a low humidity environment (10% relative humidity at room temperature inside a low humidity chamber) allowed to dry for at least 12 hours. Proteins were printed in batches of 24 slides. A single master plate could generate from 24 to ˜200 slides. Every 24^{th} slide was quality control imaged using Alexa Fluor anti-GST antibody. Briefly, auto-antigen array slides were blocked with 1% BSA in PBS, 0.1% Tween-20 (PBST) followed by development with 240 ng/mL rabbit anti GST Alexa Fluor 647 in PBST/0.3% BSA. The intensities of the spots were quantitated using a GenePix 4000B micro-array scanner.

The Ver.3 test chips were blocked with blocking buffer (1×PBS, 1% BSA, 0.1% Tween-20) for 1 hr at 4 degrees. The blocking was done in glass staining dish with gentle agitation. The serum was diluted 1:150 in probing buffer (1×PBS, 5 mM Mgcl₂, 0.5 mM DTT, 0.05% Triton X-100, 5% glycerol, 1% BSA). 100 ul of diluted serum was then applied to the lifterslip (Erie Scientific) to probe the protein array chip with autoantibodies. The lifterslip was applied to cover the chip from one end to the other so that no bubbles were trapped between the lifeterslip and the chip. The serum probing was done at 4 degrees for one and half hours in a moisture chamber (Evergreen, cat #240-9020-Z10). After serum probing, the chips were washed three times with the probing buffer (10 min/wash) in a pap jar (1 slide per pap jar in 25 ml volume, Evergreen cat #222-5450-G8S). The washed chips were then incubated with secondary antibody goat anti-human IgG labeled with Alexa Fluo 647 (Invitrogen, cat #A21445) for one and half hours at dilution 1:2000 in the probing buffer. The chips were then washed three times (10 min/wash, in pap jar with 25 ml volume) with probing buffer and spin dried. The chips were scanned with an Axon scanner (PMT 600, 33% power).

The antigen content and experimental design methodologies described above were carried out using serum samples of 19 biopsy-verified prostate-cancer (PCa) patient serum samples and 12 benign hypertrophy (BPH) patient serum samples. One of the main complicating factors in the diagnosis of prostate cancer using PSA as a biomarker, is PSA's inability to discriminate between bone fide' prostate cancer and a non-life threatening condition termed benign prostatic hypertrophy (BPH). Hence, to generate a biomedically relevant autoantibody signature for PCa, the autoantibody detection chips were screened with serum from biopsy-verified PCa and biopsy-verified BPH patients. The serum was in all cases collected before digital rectal examination and before prostate biopsy was performed, such that the results obtained were not dependent on or influenced by having a DRE or biopsy procedure.

Data analysis was performed on locally background subtracted data, and normalization was performed by calculating the median positive controls signal (either Protein L 1.6 ng/ml, Goat Anti-Human IgG, Human IgG 0.4 ng/ml, Human IgG 1.6 ng/ml, Mouse Anti Biotin 25 ng/ml, Mouse Anti-Human Kappa 6.3, or Mouse Anti-Human IgG1) and dividing it into the median of the antigen/antibody signal to give the normalized signal. Using this normalized signal for each marker, a logistic regression classifier, using a leave-one-out approach, was used to predicted the left out observation. This was done for each normalized signal. Once all of the normalized signals were predicted by the leave-one-out logistic regression, a Receiver Operator Characteristic (ROC) Curve was calculated, and the associated Area Under the Curve (AUC) was calculated. Marker sets were then ranked based on AUC's from largest to smallest. For each marker set, using the ROC, we looked for the optimal point on the curve that maximizes both Sensitivity and Specificity.

The markers of Table 3 exhibited signals that were at least two times background. Tables 5-9 provide marker sets (autoantibody detection sets) that had greater than 80% Sensitivity and greater than 80% Specificity when analyzed by the above methods. The tables provide the positive control (“Method”) that was used to normalize the data on which the classifier was built. Tables 5 through 9 also provide designations for each classifier, or marker set (5-1, 5-2, 6-1, 6-2, etc.) and subsequent columns in Tables 5 through 9 provide the markers of each autoantibody detection set. In Tables 5b, 6b, 7b, 8b, and 9b, statistical values are provided for each marker set. “AUC” is the area under the curve of the associated leave-one-out Receiver Operator Characteristic (“ROC”) curve for the classifier. “Specificity” is the frequentist estimate of the specificity of the classifier based on testing and analysis of 19 prostate cancer serum samples and 12 BPH serum samples, i.e., the percentage of negatives (BPH patients) correctly identified. “Sensitivity” is the frequentist estimate of the sensitivity of the classifier based on testing and analysis of 19 prostate cancer serum samples and 12 BPH serum samples, i.e., the percentage of positives (prostate cancer patients) correctly identified. “Bayesian Specificity” is the Bayesian estimate of the specificity of the classifier based on testing and analysis of 19 prostate cancer serum samples and 12 BPH serum samples, i.e., the sum of the number of correctly identified negatives (BPH patients) and one, divided by the sum of the number of BPH samples and two. “Bayesian Sensitivity” is the Bayesian estimate of the sensitivity of the classifier based on testing and analysis of 19 prostate cancer serum samples and 12 BPH serum samples, i.e., the sum of the number of correctly identified positives (prostate cancer patients) and one, divided by the sum of the number of prostate cancer samples and two. “Bayesian Accuracy” is the Bayesian estimate of the accuracy of the classifier based on testing and analysis of 19 prostate cancer serum samples and 12 BPH serum samples, i.e., the sum of the number of correctly identified samples and one, divided by the sum of the number of samples and two.

Three-marker autoantibody detection sets having greater than 80% Bayesian Accuracy are provided in Table 5. Four-marker autoantibody detection sets having greater than 80% Bayesian Accuracy are provided in Table 6. Five-marker autoantibody detection sets having greater than 80% Bayesian Accuracy are provided in Table 7. Six-marker autoantibody detection sets having greater than 80% Bayesian Accuracy are provided in Table 8. Seven-marker autoantibody detection sets having greater than 80% Bayesian Accuracy are provided in Table 9. Four of the seven-marker autoantibody detection sets exhibited 100% Specificity and 100% Sensitivity for distinguishing prostate cancer from BPH. These autoantibody detection sets were: Set 7-1: HEYL, BDKRB2, MAD1L1, PSAP, CCNA1, ERG, and PCNA; Set 7-2: HEYL, BDKRB2, PSAP, CCNA1, ERG, IMP-3, and PCNA; Set 7-3: HEYL, BDKRB2, PSAP, CCNA1, ERG, PIM1, and PCNA; and Set 7-4: HEYL, BDKRB2, PSAP, CCNA1, EDS, RDH11, and PCNA. Each of these biomarker detection sets had a calculated ROC/AUC of 1.

Individual markers (autoantibody capture molecules) that were present in at least 10% of two or more of the statistically significant classifiers of Tables 5, 6, 7, 8, and 9 are provided in Table 10. In column 2 of Table 10, the number of normalization techniques (out of seven) in which the marker (autoantibody capture molecule) was present in 10% or more of the identified statistically significant classifiers is provided. In column 3, the percentage of normalization techniques in which the (autoantibody capture molecule) was present in 10% or more of the identified statistically significant classifiers is provided.

### Example 2

### Identification of Autoantigens Present in Prostate Cancer Sera on ProtoArrav™ Human Protein Array

The human Protoarray™ high content protein microarray from Invitrogen (Carlsbad, Calif.) was screened with sera using the methods provided in Example 1. A combination of single-patient sample and pooled-patient samples were utilized with these arrays. A total of 32 patient samples were screened (16 prostate cancer and 16 BPH) as well as series of pooled-patient samples representing high, medium, and low PSA values. All of this data was analyzed together to generate a list of approximately 98 candidate prostate cancer biomarkers (Table 11a).

The high density Protoarray™ microarray data was normalized using a Quantile Normalization method for all chips used. After normalization, the diagnostic value of individual markers was estimated by calculating all possible order M-statistics and their associated p-values. The order with the lowest p-value was selected for each marker, the prevalence of the marker was calculated using a standard Bayesian estimate of prevalence. Markers with p-value less then 0.002 were determined as significance. For the pooled data analysis the ratio of Low Grade PCa versus BPH and High Grade PCa versus BPH was calculated, any marker that showed at least a 20% increase or decrease in signal was considered significant. Only markers that were determined to be significant in both the normal ProtoArray Analysis and the pooled experiment are provided in Table 11a.

Table 11a provides the terms used for the markers that were found to have significance for the detection of prostate cancer, high grade (HG) prostate cancer (PCA), or low grade (LG) prostate cancer over BPH. The panel also provides the Genbank identifier and/or reference sequence ID, the Invitrogen ORF designation, and the “Significance Call” of whether the marker had significance for distinguishing prostate cancer overall, high grade prostate cancer, or low grade prostate cancer from BPH. Table 11b provides in one column the “Low Grade Cancer/Normal Ratio”, which is the ratio of Low Grade Pool signal to BPH pooled signal; in another column the “High Grade Cancer/Normal Ratio”, which is the ratio of High Grade Pool signal to BPH pooled signal; in another column the “All PCA vs BPH P-Value”, which is the p-value of the difference between all PCa versus BPH in the individual analysis; in another column the “HG PCA vs BPH P-Value”, which is the p-value of the difference between High Grade PCa versus BPH in the individual analysis; and in a final column of the panel the “LG PCA vs BPH P-Value”, which is the p-value of the difference between Low Grade PCa vs BPH in the individual analysis (p-values are based on M-Statistics). Table 11c, one column provides the BPH Prevalence, or the estimated Bayesian prevalence of the marker in all BPH samples based on the signals of the individual protoarray experiments; in another column is provided the “All PCA Prevalence” which is the estimated Bayesian prevalence of the marker in all prostate cancer samples based on the signals of the individual protoarray experiments; in another column is provided the “HG PCA Prevalence” which is the estimated Bayesian prevalence of the marker in all High Grade prostate cancer samples based on the signals of the individual protoarray experiments; and a final column of the third panel provides the “LG PCA Prevalence”, which is the estimated Bayesian prevalence of the marker in all Low Grade prostate cancer samples based on the signals of the individual protoarray experiments.

### Example 3

### Autoantibodies Differentiating Prostate Cancer from Benign Prostatic Hyperplasia in Patients

Autoantibody profiling using a protein microarray chip containing 96 proteins thought to be associated with prostate cancer development was conducted using sera from 32 patients with prostate cancer and 32 patients with benign prostatic hyperplasia. The goal was to find biomarkers that are stable in blood, easily measured using approximately 1 μL of serum (or plasma), and that can differentiate true prostate cancer from the closely-related benign prostatic hyperplasia, the major weakness in the current, clinically used, PSA-based prostate cancer diagnostic test.

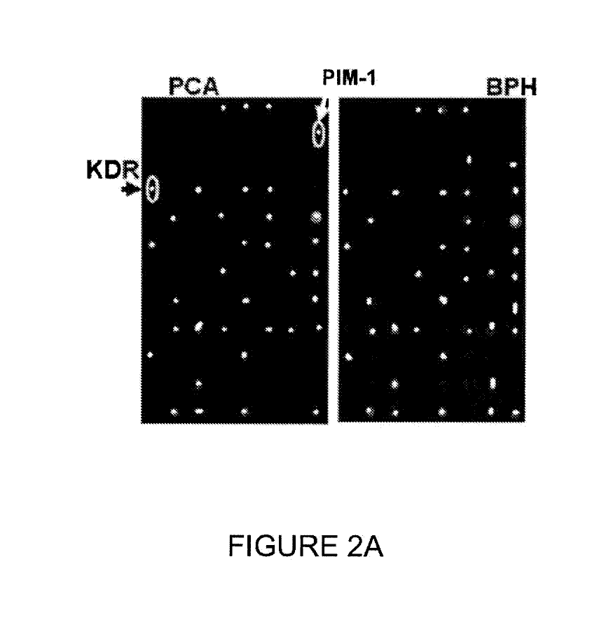

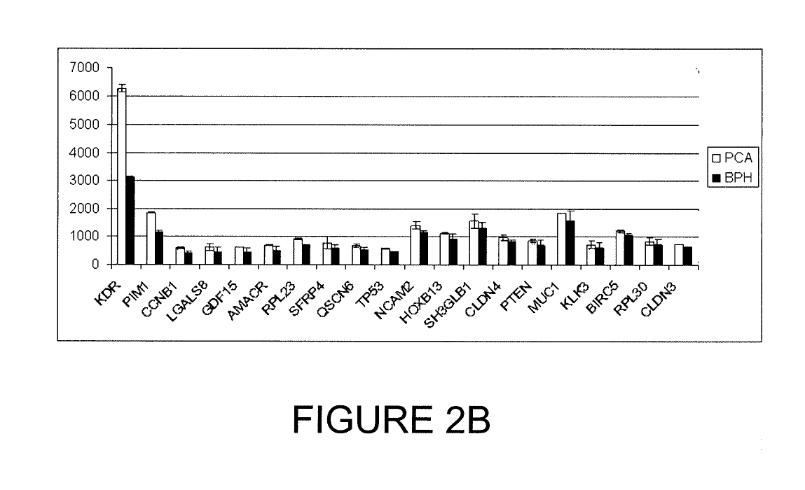

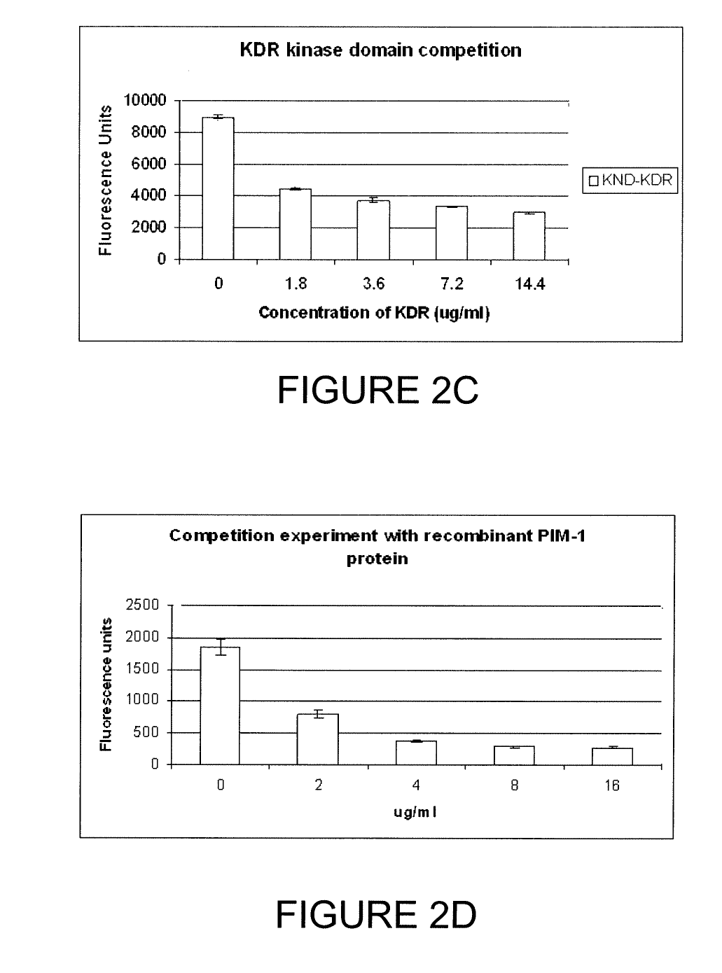

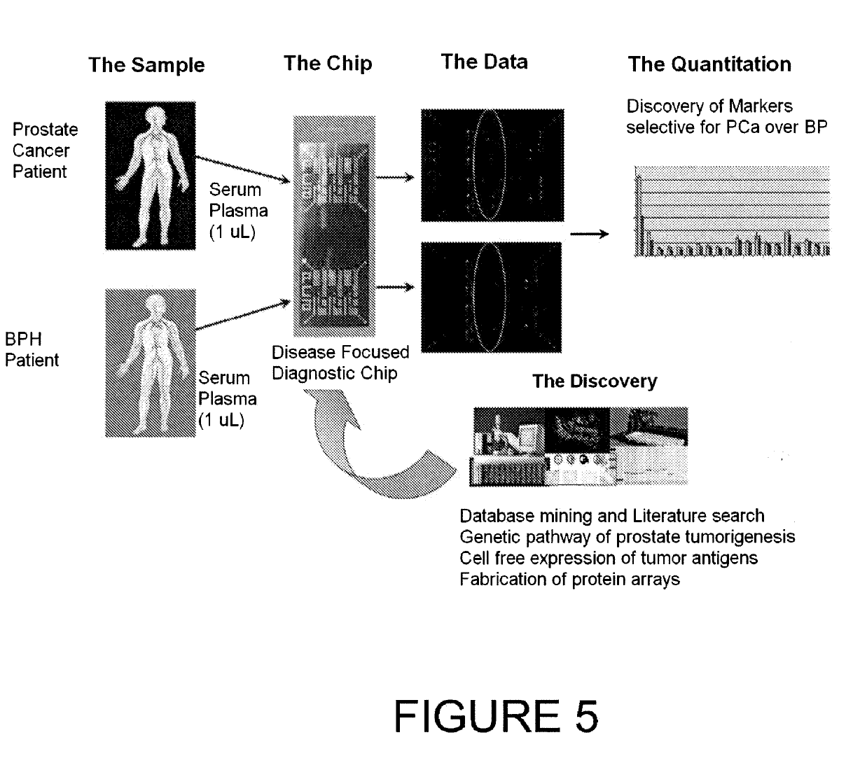

The scheme for testing chips with human sera from individuals with prostate cancer and BPH is provided in FIG. 5. Serum samples from individuals having prostate cancer and BPH were collected and contacted with a chip containing the possible target antigens. The resulting binding of target antigens to autoantibodies was quantified and used to identify the biomarkers selective for prostate cancer over BPH. The top 20 biomarkers that had significant difference between pooled PCa sera and pooled BPH are shown in FIG. 2B.

The selected antigens were expressed using a cell-free expression system, purified and arrayed on microslides. The autoantibody profiling results with the pooled PCA and BPH samples showed that twenty of these protein antigens detected significant autoantibody signals which differentiate PCa from BPH. Among these twenty protein antigens, p53, CCNB1, survivin, and mucin1 are common tumor associated antigens shared by various cancer types such as breast cancer, colon cancer, prostate cancer, lung cancer and melanoma, but which have not been previously utilized as part of a prostate cancer diagnostic assay. Fourteen of these antigens are completely novel tumor antigens which have not been reported before.

In particular, two novel tumor antigens, KDR and PIM-1 which contribute to PCa development and progression, were shown to have significant sensitivity and specificity with regard to PCa diagnosis. KDR is a type III receptor tyrosine kinase which is involved in the angiogenesis and proliferation of PCa (Huss et al. (2001) “Angiogenesis and prostate cancer: identification of a molecular progression switch,” Cancer Res. 61(6):2736-43; Jackson et al. (2002) “A potential autocrine role for vascular endothelial growth factor in prostate cancer,” Cancer Res. 62(3):854-9; and Soker et al. (2001) “Vascular endothelial growth factor-mediated autocrine stimulation of prostate tumor cells coincides with progression to a malignant phenotype,” Am J. Pathol. 159(2):651-9). PIM-1 is a serine/threonine kinase and its over expression in the prostate leads to the genomic instability which contributes to the tumor progression (Valdman et al. (2004) “Pim-1 expression in prostatic intraepithelial neoplasia and human prostatecancer,” Prostate. 60(4):367-71; Bhattacharya et al. (2002) “Pim-1 associates with protein complexes necessary for mitosis,” Chromosoma. 111(2):80-95; Roh et al. (2003) “Overexpression of the oncogenic kinase Pim-1 leads to genomic instability,” Cancer Res. 63(23):8079-84; and Cibull et al. (2006) “Overexpression of Pim-1 during progression of prostatic adenocarcinoma,” J. Clin. Pathol. 59(3):285-8).

Autoantibodies against KDR and PIM-1 were shown to be present in ˜62% PCA patients and ˜30% BPH patients. Furthermore the pairing of KDR and PIM-1 confers a sensitivity of 90.6% and specificity of 84.4% in diagnosing PCa over an equal number of BPH samples. The tissue microarray experiments indicated that KDR and PIM-1 antigens are over expressed in 70% and 30% PCa patients respectively suggesting that over expression of these tumor antigens may account for the aberrant autoantibody induction.

Interestingly, in PCa, KDR and PIM-1 autoantibodies were very effective in diagnosing with small size tumors (tumors with 1-2 positive cores as pathologically diagnosed) where a PSA assay had limited diagnostic value.

It is worth noting that this study was performed using very small amounts of cell-free-extract in-vitro synthesized proteins (˜10 μg). All that is required to make these antigens is the requisite open-reading-frame genetic constructs combined with a cell-free expression system; both materials are widely commercially available. Autoantibodies discovered in this manner are of very high diagnostic quality because they stably exist in sera. Only 1 μL of sera is required for the assay and the diagnostic-assay development step itself is eliminated. Moreover, generic detection reagents (e.g., fluorescently labeled Goat anti-human antibodies) can be used for detection on the protein chip.

### Materials and Methods

Patients and Sera. Sixty four biopsy-proven serum samples from thirty two untreated PCA patients and thirty two untreated BPH patients were collected by BIOCHEMED Corporation (1483 Tobias Blvd., Charles, S.C. 29407) before the patients had DRE and biopsies. All samples were tested for PSA level by Beckman Access (Fullerton, Calif.). A complete medical history was provided for each identified patient with biopsy information on a patient's Gleason scores, number of positive scores for all 8 needle biopsy cores, as well as the percentage of cancer cells in a single core. All samples were collected from patients in the South Carolina area with informed consent forms and the studies were approved by the institutional review boards. Patient information is listed in Table 12.

TABLE 12
Clinical data for prostate cancer and benign prostatic hyperplasia patients

| | Prostate | Benign Prostatic |
|---|---|---|
| Variables | Cancer Set | Hyperplasia Set |
| No of patients | 32 | 32 |
| Mean age ± SD | 65.17 ± 7.79 | 61.72 ± 9.24 |
| PSA level | | |
| <4 ng/ml | 37.5% | 50% |
| 4-10 ng/ml | 50% | 37.5% |
| >10 ng/ml | 12.5% | 12.5% |
| Gleason Score | | |
| <6 | 72% | NA |
| >7 | 28% | NA |

Expression of tumor antigens and fabrication of antigen microarray. The 96 recombinant GST tagged proteins were obtained using an in-vitro wheat germ cell-free expression system (Abnova or Invitrogen) and are shown in Table 13. Cell-free expressed proteins were eluted from GST-columns (Invitrogen, cat# 13-6741) with Tris-Glutathione buffer (pH 8.0). The proteins were expressed in either full-length or truncated forms. Each protein was quality controlled (for correct mass-weight and impurities) using an Agilent Bioanalyzer (Santa Clara, Calif.) and then arrayed as quadruplicate on one-pad nitrocellulose slide (Whatman, cat# 10484182) at concentrations ranging from about 0.1 to about 0.25 μg/ml using a sciFLEXARRAYER S5 Piezo Dispenser (Scienion AG, Berlin, Germany). A low content chip containing KDR and PIM-1 was made on a 16-pad nitrocellulose slide (Whatman, cat# 10485323) in which each protein was printed in duplicate, and each chip contained 12 total arrays. Mouse anti-human IgG1 (Invitrogen, cat# 05-3300) was printed as four identical spots of 6 μg/ml on each array and was used for the normalization of microarray signals. Select microarray slides from each printing run were probed with anti-GST antibodies to measure the final amount of printed protein on each spot on the array.

TABLE 13
Function and source of protein antigens used for autoantibody profiling chip

| HUGO | Protein | Protein/Gene | GenBank | | Biomarker |
|---|---|---|---|---|---|
| Designation | source | Aliases | Identifiers | Function | Grouping |
| ABL1 | ABL1 (full- | JTK7, c-ABL, | v-abl Abelson | Oncogene | Apoptosis |
| | length) | p150, v-abl | murine leukemia | | |
| | Invitrogen | | viral oncogene | | |
| | | | homolog 1 | | |
| | | | NM_007313 | | |
| ACPP | ACPP | ACPP, PAP, | Acid phosphatase, | Catalyze | Cellular |
| | (310-418) | ACP3, ACP-3 | prostate | phosphate | metabolism |
| | Abnova | | NM_001099 | monoester into | |
| | | | | alcohol and | |
| | | | | phosphate. | |
| AGR2 | AGR2 | AGR2, AG2, | Anterior gradient | Cell | Metastasis |
| | (full- | GOB-4, HAG- | homolog 2 | differentiation | |
| | length) | 2, | (Xenopus laevis) | | |
| | Abnova | XAG-2 | NM_006408 | | |
| AKT1 | AKT1 (full- | AKT1, PKB, | v-akt murine | Oncogene | Apoptosis |
| | length) | RAC, | thymoma viral | | |
| | Invitrogen | PRKBA, | oncogene | | |
| | | MGC99656, | homolog 1 | | |
| | | RAC-ALPHA | NM_005163 | | |
| ALOX15 | ALOX15 | ALOX15 | Arachidonate 15- | Converts | Cellular |
| | (full- | | lipoxygenase | arachidonic | metabolism |
| | length) | | NM_1140 | acid to 15S- | |
| | Abnova | | | hydro- | |
| | | | | peroxyeicosate | |
| | | | | traenoic acid | |
| AMACR | AMACR | AMACR, | Alpha-methylacyl- | Racemization | Cellular |
| | (full- | RACE | CoA racemase | of 2-methyl- | metabolism |
| | length) | | NM_014707 | branched fatty | |
| | Abnova | | | acid CoA | |
| | | | | esters | |
| BCL2 | BCL2 | BCL2, Bcl-2 | B-cell | Suppresses | Apoptosis |
| | (140-239) | | CLL/lymphoma 2 | apoptosis | |
| | Abnova | | NM_000633 | | |
| BCL2L14 | BCL2L14 | BCL2L14, | BCL2-like 14 | Apoptosis | Apoptosis |
| | (full- | BCLG | NM_030766 | facilitator | |
| | length) | | | | |
| | Abnova | | | | |
| BDKRB2 | BDKRB2 | BDKRB2, | Bradykinin | Receptor for | Cell surface |
| | (1-61) | B2R, | receptor B2 | bradykinin | protein |
| | Abnova | BK2, BK-2, | NM_000623 | | |
| | | BKR2, BRB2, | | | |
| | | DKFZp686O088 | | | |
| BIRC5 | BIRC5 | BIRC5, API4, | Baculoviral IAP | Anti-apoptotic | Apoptosis |
| | (full- | EPR-1 | repeat- | | |
| | length) | | containing 5 | | |
| | Abnova | | (survivin) | | |
| | | | NM_001012270 | | |
| CAV3 | CAV3 (1-84) | CAV3, VIP21, | Caveolin 3 | Scaffolding | Tumor |
| | Abnova | LGMD1C, | NM_001753 | protein within | suppressor |
| | | VIP-21, | | caveolar | |
| | | MGC126100, | | membranes | |
| | | MGC126101, | | | |
| | | MGC126129 | | | |
| CCKBR | CCKBR | CCKBR, | Cholecystokinin B | Receptor for | Cell surface |
| | (215-327) | GASR, | Receptor | gastrin and | protein |
| | Abnova | CCK-B | NM_176875 | cholecystokinin | |
| CCNA1 | CCNA1 | CCNA1 | Cyclin A1 | Cell cycle | Cell cycle |
| | (full- | | NM_003914 | regulation | |
| | length) | | | | |
| | Abnova | | | | |
| CCNB1 | CCNB1 (1-91) | CCNB1, | Cyclin B1 | Cell cycle | Cell cycle |
| | Abnova | CCNB | NM_031966 | regulation | |
| CCND1 | CCND1 | CCND1, | Cyclin D1 | Cell cycle | Cell cycle |
| | (full- | BCL1, | NM_053056 | regulation | |
| | length) | PRAD1, U21B31, | | | |
| | Abnova | D11S287E | | | |
| CD151 | CD151 | CD151, | CD151 molecule | Essential for | Metastasis |
| | (full- | GP27, | (Raph blood | the proper | |
| | length) | MER2, | group) | assembly of | |
| | Abnova | RAPH, | NM_004357 | the glomerular | |
| | | SFA1, PETA- | | and tubular | |
| | | 3, | | basement | |
| | | TSPAN24 | | membranes in | |
| | | | | kidney | |
| CD164 | CD164 (1-115) | CD164, | CD164 molecule, | Mucin-like | Metastasis |
| | Abnova | MGC-24, | sialomucin | protein | |
| | | MUC-24, | NM_006016 | | |
| | | Endolyn | | | |
| CDKN2A | CDKN2A | CDKN2A, | Cyclin-dependent | Tumor | Tumor |
| | (full- | ARF, | kinase inhibitor 2A | suppressor | suppressor |
| | length) | MLM, p14, | NM_000077 | | |
| | Abnova | p16, | | | |
| | | p19, CMM2, | | | |
| | | INK4, MTS1, | | | |
| | | TP16, CDK4I, | | | |
| | | CDKN2, | | | |
| | | INK4a, | | | |
| | | p14ARF, | | | |
| | | p16INK4, | | | |
| | | p16INK4a | | | |
| CHEK1 | CHK1 (full- | CHK1 | CHK1 checkpoint | Cell cycle | Cell cycle |
| | length) | | homolog (S. pombe) | regulation | |
| | Invitrogen | | NM_001274 | | |
| CLDN3 | CLDN3 | CLDN3, | Claudin 3 | Cell adhesion | Cell surface |
| | (full- | RVP1, | NM_001306 | | protein |
| | length) | HRVP1, | | | |
| | Abnova | C7orf1, | | | |
| | | CPE-R2, | | | |
| | | CPETR2 | | | |
| CLDN4 | CLDN4 | CLDN4, | Claudin 4 | Cell adhesion | Cell surface |
| | (full- | CPER, | NM_001305 | | protein |
| | length) | CPE-R, | | | |
| | Abnova | CPETR, | | | |
| | | CPETR1, | | | |
| | | WBSCR8, | | | |
| | | hCPE-R | | | |
| CUL4A | CUL4A (1-100) | CUL4A | Cullin 4A | DNA repair | Cell cycle |
| | Abnova | | NM_003589 | | |
| CXCR4 | CXCR4 (1-47) | CXCR4, | Chemokine (C—X—C | Tumor | Metastasis |
| | Abnova | FB22, | motif) receptor 4 | metastasis | |
| | | HM89, LAP3, | NM_001008540 | | |
| | | LCR1, NPYR, | | | |
| | | WHIM, | | | |
| | | CD184, | | | |
| | | LESTR, | | | |
| | | NPY3R, | | | |
| | | NPYRL, | | | |
| | | HSY3RR, | | | |
| | | NPYY3R, | | | |
| | | D2S201E | | | |
| EDNRB | EDNRB | EDNRB, | Endothelin | Non-specific | Cell surface |
| | (27-101) | ETB, | receptor type B | receptor for | protein |
| | Abnova | ETRB, | NM_000115 | endothelin 1, 2, | |
| | | HSCR, | | and 3 | |
| | | ABCDS, | | | |
| | | HSCR2 | | | |
| EGFR | EGFR (26-126) | EGFR, | Epidermal growth | Growth factor | Cell surface |
| | Abnova | ERBB, | factor receptor | receptor | protein |
| | | mENA, | (erythroblastic | | |
| | | ERBB1 | leukemia viral (v- | | |
| | | | erb-b) oncogene | | |
| | | | homolog, avian) | | |
| | | | NM_005228 | | |
| EIF3S3 | EIF3S3 | EIF3S3, | Eukaryotic | Protein | Cellular |
| | (full- | eIF3-p40, | translation | translation | metabolism |
| | length) | MGC102958, | initiation factor 3, | | |
| | Abnova | eIF3-gamma | subunit 3 gamma, | | |
| | | | 40 kDa | | |
| | | | NM_003756 | | |
| ELAC1 | ELAC1 | ELAC1, D29 | elaC homolog 1 | Involved in | Cellular |
| | (282-363) | | (E. coli) | tRNA | metabolism |
| | Abnova | | NM_018696 | maturation | |
| ENO1 | ENO1 | ENO1, NNE, | Enolase 1, (alpha) | Plays a part in | Cell growth |
| | (full- | PPH, MPB1, | NM_001428 | various | |
| | length) | MBP-1, | | processes such | |
| | Abnova | ENO1L1 | | as growth | |
| | | | | control, | |
| | | | | hypoxia | |
| | | | | tolerance and | |
| | | | | allergic | |
| | | | | responses | |
| ENOX2 | COVA1 | COVA1, | Ecto-NOX | Cell growth, | Cell surface |
| | (full- | APK1, | disulfide-thiol | tumor antigen | protein |
| | length) | tNOX | exchanger 2 | | |
| | Abnova | | NM_006375 | | |
| ERBB2 | ERBB2 | ERBB2, NEU, | v-erb-b2 | Tyrosine | Cell surface |
| | (676-1255) | NGL, HER2, | erythroblastic | kinase-type cell | protein |
| | Invitrogen | TKR1, HER- | leukemia viral | surface | |
| | | 2, | oncogene | receptor HER2, | |
| | | c-erb B2, | homolog 2, | Oncogene | |
| | | HER-2/neu | neuro/glioblastoma | | |
| | | | derived oncogene | | |
| | | | homolog (avian) | | |
| | | | NM_001005862 | | |
| ERG | ERG (full- | ERG, p55, | v-ets | Oncogene | Cell growth and |
| | length) | erg-3 | erythroblastosis | | differentiation |
| | Abnova | | virus E26 | | |
| | | | oncogene | | |
| | | | homolog (avian) | | |
| | | | NM_004449 | | |
| ETS2 | ETS2 (1-101) | ETS2 | v-ets | Oncogene | Cell growth and |
| | Abnova | | erythroblastosis | | differentiation |
| | | | virus E26 | | |
| | | | oncogene | | |
| | | | homolog 2 (avian) | | |
| | | | NM_005228 | | |
| EZH2 | EZH2 (full- | EZH1, ENX-1 | Enhancer of zeste | Involved in the | Metastasis |
| | length) | | homolog 2 | regulation of | |
| | Abnova | | (Drosophila) | gene | |
| | | | NM_004456 | transcription | |
| | | | | and chromatin | |
| | | | | structure. | |
| FASN | FASN (full- | FASN, FAS, | Fatty acid | Fatty acid | Cellular |
| | length) | OA-519, | synthase | metabolism | metabolism |
| | Abnova | MGC14367, | NM_004104 | | |
| | | MGC15706 | | | |
| FLT1 | FLT1 | FLT1, FLT, | Fms-related | Involved in | Angiogenesis |
| | (aa781-1338) | VEGFR1 | tyrosine kinase 1 | angiogenesis | |
| | Invitrogen | | (vascular | | |
| | | | endothelial growth | | |
| | | | factor/vascular | | |
| | | | permeability factor | | |
| | | | receptor) | | |
| | | | NM_002019 | | |
| FOLH1 | FOLH1 | FOLH1, PSM, | Folate hydrolase | Has both folate | Cell surface |
| | (547-657) | FGCP, | (prostate-specific | hydrolase and | protein |
| | Abnova | FOLH, | membrane | N-acetylated- | Cellular |
| | | GCP2, | antigen) 1 | alpha-linked- | metabolism |
| | | PSMA, | NM_004476 | acidic | |
| | | mGCP, | | dipeptidase | |
| | | GCPII, | | (NAALADase) | |
| | | NAALAD1, | | activity. | |
| | | NAALAdase | | | |
| GDF15 | GDF15 | GDF15, PDF, | Growth | Cell growth | Cell growth |
| | (full- | MIC1, PLAB, | differentiation | factor | |
| | length) | MIC-1, NAG- | factor 15 | | |
| | Abnova | 1, | NM_004864 | | |
| | | PTGFB, | | | |
| | | GDF-15 | | | |
| HEYL | HEYL (full- | HEYL, HRT3, | Hairy/enhancer-of- | Transcriptional | Angiogenesis |
| | length) | MGC12623 | split related with | repressor | |
| | Abnova | | YRPW motif-like | | |
| | | | NM_014571 | | |
| HIPK3 | HIPK3 | PKY, DYRK6, | Homeodomain | Regulate | Apoptosis |
| | (163-562) | YAK1 | interacting protein | apoptosis by | |
| | Invitrogen | | kinase 3 | promoting | |
| | | | NM_001048200 | FADD | |
| | | | | phosphorylation | |
| HMGA2 | HMGA2 | HMGA2, | High mobility | Transcription | Cell growth and |
| | (1-93) | BABL, | group AT-hook 2 | regulation | differentiation |
| | Abnova | LIPO, | NM_003484 | | |
| | | HMGIC, | | | |
| | | HMGI-C | | | |
| HOXB13 | HOXB13 | HOXB13, | Homeobox B13 | Transcription | Cell |
| | (full- | PSGD | NM_006361 | factor involved | differentiation |
| | length) | | | in cell | |
| | Abnova | | | differentiation | |
| HPN | HPN (full- | HPN, | Hepsin | Cell growth | Cell growth |
| | length) | TMPRSS1 | (transmembrane | | |
| | Abnova | | protease, serine 1) | | |
| | | | NM_002151 | | |
| HSPA5 | HSPA5 | HSPA5, BIP, | Heat shock 70 kDa | Stress | Stress |
| | (full- | MIF2, | protein 5 (glucose- | response | response |
| | length) | GRP78, | regulated protein, | protein | |
| | Abnova | FLJ26106 | 78 kDa) | | |
| | | | NM_005347 | | |
| HSPD1 | HSPD1 | HSPD1, | Heat shock 60 kDa | Stress | Stress |
| | (full- | CPN60, | protein 1 | response | response |
| | length) | GROEL, | (chaperonin) | protein | |
| | Abnova | HSP60, | NM_002156 | | |
| | | HSP65, | | | |
| | | SPG13, | | | |
| | | HuCHA60 | | | |
| KDR | KDR (789-1356) | KDR, FLK1, | Kinase insert | Angiogenesis | Angiogenesis |
| | Invitrogen | CD309, | domain receptor (a | | |
| | | VEGFR, | type III receptor | | |
| | | VEGFR2 | tyrosine kinase) | | |
| | | | NM_002253 | | |
| KLK3 | PSA (full- | KLK3, APS, | Kallikrein 3, | Protease | Angiogenesis |
| | length) | PSA, hK3, | (prostate specific | | |
| | Abnova | KLK2A1 | antigen) | | |
| | | | NM_145864 | | |
| LGALS8 | LGALS8 | LGALS8, Gal- | Lectin, | Cell adhesion | Cell surface |
| | (full- | 8, | galactoside- | | protein |
| | length) | PCTA1, | binding, soluble, 8 | | |
| | Abnova | PCTA-1, | (galectin 8) | | |
| | | Po66-CBP | NM_006499 | | |
| MAD1L1 | MAD1L1 | MAD1L1, | MAD2 mitotic | Cell division | Cell cycle |
| | (619-718) | MAD1, PIG9, | arrest deficient-like 1 | | |
| | Abnova | HsMAD1, | NM_002358 | | |
| | | TP53I9, | | | |
| | | TXBP181 | | | |
| MDM2 | MDM2 | MDM2, | Mdm2, | Cell cycle | Cell cycle |
| | (101-201) | hdm2, | transformed 3T3 | regulation | |
| | Abnova | MGC71221 | cell double minute | | |
| | | | 2, p53 binding | | |
| | | | protein | | |
| | | | NM_002392 | | |
| MET | MET (26-125) | MET, HGFR, | Met proto- | Oncogene | Apoptosis |
| | Abnova | RCCP2 | oncogene | | |
| | | | (hepatocyte | | |
| | | | growth factor | | |
| | | | receptor) | | |
| | | | NM_000245 | | |
| MIB1 | MIB1 | MIB1, MIB, | Mindbomb | E3 ubiquitin- | Cell cycle |
| | (909-1007) | ZZZ6, DIP-1, | homolog 1 | protein ligase | |
| | Abnova | ZZANK2, | (Drosophila) | that mediates | |
| | | FLJ90676, | NM_020774 | ubiquitination | |
| | | MGC129659, | | of Delta | |
| | | MGC129660, | | receptors, | |
| | | DKFZp686I0769 | | which act as | |
| | | | | ligands of | |
| | | | | Notch proteins | |
| MICB | MICB (full- | MICB, | MHC class I | Ligand for NK | Innate |
| | length) | PERB11.2 | polypeptide- | cells | Immunity |
| | Abnova | | related sequence B | | |
| | | | NM_005931 | | |
| MLH1 | MLH1 (full- | MLH1, FCC2, | mutL homolog 1, | DNA mismatch | Cell cycle |
| | length) | COCA2, | colon cancer, | repair | |
| | Abnova | HNPCC, | nonpolyposis type 2 | | |
| | | hMLH1, | NM_000249 | | |
| | | HNPCC2, | | | |
| | | MGC5172 | | | |
| MMP9 | MMP9 | MMP9, | Matrix | Tumor | Metastasis |
| | (full- | GELB, | metallopeptidase 9 | metastasis | |
| | length) | CLG4B, | (gelatinase B, | | |
| | Abnova | MMP-9 | 92 kDa gelatinase, | | |
| | | | 92 kDa type IV | | |
| | | | collagenase) | | |
| | | | NM_004994 | | |
| MUC1 | MUC1 | MUC1, EMA, | Mucin 1, cell | Cell adhesion | Metastasis |
| | (315-420) | PEM, PUM, | surface associated | and tumor | |
| | Abnova | MAM6, | NM_002456 | metastasis | |
| | | PEMT, | | | |
| | | CD227, | | | |
| | | H23DG | | | |
| MYC | MYC (330-440) | MYC, c-Myc | v-myc | Oncogene | Cell cycle |
| | Abnova | | myelocytomatosis | | |
| | | | viral oncogene | | |
| | | | homolog 1, lung | | |
| | | | carcinoma derived | | |
| | | | (avian) | | |
| | | | NM_002467 | | |
| NCAM2 | NCAM2 | NCAM2, | Neural cell | Cell adhesion | Cell surface |
| | (598-696) | NCAM21, | adhesion molecule 2 | | Protein |
| | Abnova | MGC51008 | NM_004540 | | |
| NKX3-1 | NKX3-1 | NKX3-1, | NK3 transcription | Tumor | Tumor |
| | (100-210) | BAPX2 | factor related, | suppressor | suppressor |
| | Abnova | | locus 1 | specific to | |
| | | | (Drosophila) | prostate cancer | |
| | | | NM_006167 | | |
| NRP1 | NRP1 (full- | NRP1, NRP, | Neuropilin 1 | Tumor | Angiogenesis |
| | length) | CD304, | NM_015022 | angiogenesis | |
| | Abnova | VEGF165R, | | | |
| NUCB1 | NUCB1 | NUCB1, | Nucleobindin 1 | Major calcium- | Cellular |
| | (full- | NUC, | NM_006184 | binding protein | Metabolism |
| | length) | FLJ40471, | | of the Golgi | |
| | Abnova | | | | |
| PCNA | PCNA | PCNA, | Proliferating cell | DNA | Cell cycle |
| | (full- | MGC8367 | nuclear antigen | replication | |
| | length) | | NM_182649 | | |
| | Abnova | | | | |
| PDLIM1 | PDLIM1 | PDLIM1, | PDZ and LIM | Cytoskeletal | Cell growth and |
| | (123-233) | CLIM1, | domain 1 (elfin) | protein that | Differentiation |
| | Abnova | CLP36, | NM_020992 | may act as an | |
| | | ELFIN, CLP- | | adapter that | |
| | | 36, hCLIM1 | | brings other | |
| | | | | proteins (like | |
| | | | | kinases) to the | |
| | | | | cytoskeleton | |
| PECAM1 | PECAM1 | PECAM1, | Platelet/endothelial | Cell adhesion | Cell surface |
| | (full- | CD31, | cell adhesion | | protein |
| | length) | PECAM-1 | molecule (CD31 | | |
| | Abnova | | antigen) | | |
| | | | NM_000442 | | |
| PIM-1 | PIM-1(full- | PIM-1, PIM | pim-1 oncogene | Oncogene | Cell cycle |
| | length) | | NM_02648 | | |
| | Abnova | | | | |
| PRSS8 | PRSS8 | PRSS8, | Homo sapiens | Possesses a | Cell growth and |
| | (full- | CAP1, | protease, serine, 8 | trypsin-like | Differentiation |
| | length) | PROSTASIN | (prostasin) | cleavage | |
| | Abnova | | NM_002773 | specificity. | |
| PSAP | PSAP (full- | PSAP, GLBA, | Prosaposin | Lipid | Cellular |
| | length) | SAP1, | (variant Gaucher | metabolism | Metabolism |
| | Abnova | FLJ00245, | disease and | | |
| | | MGC110993 | variant | | |
| | | | metachromatic | | |
| | | | leukodystrophy) | | |
| | | | NM_002778 | | |
| PSCA | PSCA (23-96) | PSCA, | Prostate stem cell | Unknown | Cell surface |
| | Abnova | PRO232 | antigen | | Protein |
| | | | NM_005672 | | |
| PSMB4 | PSMB4 | PSMB4, HN3, | Proteasome | Involved in | Cellular |
| | (full- | HsN3, | (prosome, | proteolytic | Metabolism |
| | length) | PROS26 | macropain) | activity | |
| | Abnova | | subunit, beta type, 4 | | |
| | | | NM_002796 | | |
| PTEN | PTEN | PTEN, BZS, | Phosphatase and | Tumor | Tumor |
| | (221-320) | MHAM, | tensin homolog | suppressor | Suppressor |
| | Abnova | TEP1 | (mutated in | | |
| | | MMAC1, | multiple advanced | | |
| | | PTEN1, | cancers 1) | | |
| | | MGC11227 | NM_000314 | | |
| PTGER3 | PTGER3 | PTGER3, | Prostaglandin E | GPCR | Metastasis |
| | (1-90) | EP3, | receptor 2 | receptor | |
| | Abnova | EP3e, EP3-I, | NM_000956 | involved in | |
| | | EP3-II, EP3- | | tumor | |
| | | IV, | | metastasis | |
| | | EP3-III, | | | |
| | | MGC27302, | | | |
| | | MGC141828, | | | |
| | | MGC141829 | | | |
| PTGS1 | PTGS1(26-125) | PTGS1, | Prostaglandin- | Cell | Cellular |
| | Abnova | COX1, | endoperoxide | proliferation | Metabolism |
| | | COX3, PHS1, | synthase 1 | | |
| | | PCOX1, | (prostaglandin G/H | | |
| | | PGHS1, | synthase and | | |
| | | PTGHS, | cyclooxygenase) | | |
| | | PGG/HS, | NM_000962 | | |
| | | PGHS-1 | | | |
| QSOX1 | QSCN6 | QSCN6, Q6, | Quiescin Q6 | Cell cycle | Cell cycle |
| | (81-181) | QSOX1 | NM_002826 | regulation | |
| | Abnova | | | | |
| RASSF1 | RASSF1 | RASSF1, | Ras association | Potential tumor | Tumor |
| | (241-341) | 123F2, | (RalGDS/AF-6) | suppressor | Suppressor |
| | Abnova | RDA32, | domain | | |
| | | NORE2A, | family 1 | | |
| | | RASSF1A, | NM_007182 | | |
| | | REH3P21 | | | |
| RCVRN | RCV1 | RCVRN, | Recoverin | Involved in the | Cellular |
| | (101-200) | RCV1 | NM_002903 | inhibition of the | Metabolism |
| | Abnova | | | phosphorylation | |
| | | | | of rhodopsin | |
| RDH11 | RDH11 | RDH11, | Retinol | NADPH- | Cellular |
| | (full- | MDT1, | dehydrogenase 11 | dependent | Metabolism |
| | length) | PSDR1, | (all-trans/9-cis/11- | retinal | |
| | Abnova | RALR1, | cis) | reductase | |
| | | SCALD, | NM_016026 | | |
| | | ARSDR1, | | | |
| | | CGI-82, | | | |
| | | HCBP12, | | | |
| | | FLJ32633 | | | |
| RNF14 | RNF14 | RNF14, | Ring finger protein | E3 ubiquitin- | Cellular |
| | (217-317) | ARA54, | 14 | protein ligase | Metabolism |
| | Abnova | HFB30, | NM_004290 | | |
| | | FLJ26004, | | | |
| | | HRIHFB2038 | | | |
| ROCK2 | ROCK2 | ROCK2 | Rho-associated | Protein Kinase | Cell growth |
| | (1-552) | | coiled-coil | involved in | |
| | Invitrogen | | containing protein | regulating the | |
| | | | kinase 2 | assembly of | |
| | | | NM_004850 | the actin | |
| | | | | cytoskeleton | |
| RPL23 | RPL23 | RPL23, | Ribosomal protein | Protein | Cellular |
| | (full- | rpL17, | L23 | translation | metabolism |
| | length) | MGC72008, | NM_000984 | | |
| | Abnova | MGC111167, | | | |
| | | MGC117346 | | | |
| RPL30 | RPL30 | RPL30 | Ribosomal protein | Protein | Cellular |
| | (full- | | L30 | translation | Metabolism |
| | length) | | NM_000989 | | |
| | Abnova | | | | |
| RPS14 | RPS14 | RPS14, | Ribosomal protein | Protein | Cellular |
| | (full- | EMTB | S14 | translation | Metabolism |
| | length) | | (RPS14) | | |
| | Abnova | | NM_001025070 | | |
| RPS6KA1 | RPS6KA1 | RPS6KA1, | Ribosomal protein | Mediating the | Stress |
| | (full- | RSK, | S6 | growth-factor | Response |
| | length) | HU-1, RSK1, | kinase, 90 kDa, | and stress | |
| | Invitrogen | MAPKAPK1A, | polypeptide 1 | induced | |
| | | S6K-alpha 1 | NM_002953 | activation of | |
| | | | | the | |
| | | | | transcription | |
| | | | | factor CREB. | |
| RPS6KA3 | RPS6KA3 | RSK, RSK2, | Ribosomal protein | Mediating the | Stress |
| | (full- | HU-3 | S6 | growth-factor | Response |
| | length) | | kinase, 90 kDa, | and stress | |
| | Invitrogen | | polypeptide 3 | induced | |
| | | | NM_004586 | activation of | |
| | | | | the | |
| | | | | transcription | |
| | | | | factor CREB. | |
| SERPINH1 | SERPINH1 | SERPINH1, | Serpin peptidase | Involved as a | Stress |
| | (full- | CBP1, CBP2, | inhibitor, clade H | chaperone in | Response |
| | length) | gp46, AsTP3, | (heat shock | the biosynthetic | |
| | Abnova | HSP47, | protein 47), | pathway of | |
| | | PIG14, RA- | member 1, | collagen | |
| | | A47, | (collagen binding | | |
| | | SERPINH2 | protein 1) | | |
| | | | NM_001235 | | |
| SFRP4 | SFRP4 | SFRP4, FRP- | Secreted frizzled- | Cell growth and | Cell growth and |
| | (211-313) | 4, | related protein 4 | differentiation | Differentiation |
| | Abnova | FRPHE, | NM_003014 | | |
| | | MGC26498 | | | |
| SH3GLB1 | SH3GLB1 | SH3GLB1, | SH3-domain | Apoptosis | Apoptosis |
| | (full- | Bif-1, CGI-61, | GRB2-like | | |
| | length) | KIAA0491, | endophilin B1 | | |
| | Abnova | dJ612B15.2 | NM_016009 | | |
| SPRR1B | SPRR1B | SPRR1B, | Small proline-rich | Unknown | Cell |
| | (full- | SPRR1, | protein 1B | | Differentiation |
| | length) | GADD33, | (cornifin) | | |
| | Abnova | CORNIFIN, | NM_003125 | | |
| | | MGC61901 | | | |
| STEAP1 | STEAP | STEAP1 | Six | Metalloreductase | Cell surface |
| | (full- | STEAP, | transmembrane | | Protein |
| | length) | PRSS24, | epithelial antigen | | |
| | Abnova | MGC19484 | of the prostate 1 | | |
| | | | NM_012449 | | |
| STIP1 | STIP1 | STIP1, HOP, | Stress-induced- | Stress | Stress |
| | (full- | P60, STI1L, | phosphoprotein 1 | response | Response |
| | length) | IEF-SSP- | (Hsp70/Hsp90- | | |
| | Abnova | 3521 | organizing | | |
| | | | protein) | | |
| | | | NM_006819 | | |
| TACSTD1 | EP-CAM | TACSTD1, | Tumor-associated | Cell adhesion | Cell surface |
| | (full- | EGP, | calcium signal | | Protein |
| | length) | KSA, M4S1, | transducer 1 | | |
| | Abnova | MK-1, | precursor | | |
| | | CD326, | NM_002354 | | |
| | | EGP40, | | | |
| | | MIC18, | | | |
| | | TROP1, Ep- | | | |
| | | CAM, hEGP- | | | |
| | | 2, | | | |
| | | CO17-1A, | | | |
| | | GA733-2 | | | |
| TMPRSS2 | TMPRSS2 | TMPRSS2, | Transmembrane | Cell adhesion | Angiogenesis |
| | (full- | PRSS10 | protease, serine 2 | | |
| | length) | | NM_005656 | | |
| | Abnova | | | | |
| TOP2A | TOP2A | TOP2A, | Topoisomerase | DNA replication | Cell cycle |
| | (1435-1532) | TOP2, | (DNA) II alpha | | |
| | Abnova | TP2A | 170 kDa | | |
| | | | NM_001067 | | |
| TP53 | TP53 (94-202) | TP53, p53, | Tumor protein p53 | Tumor | Tumor |
| | Abnova | LFS1, TRP53 | (Li-Fraumeni | suppressor | Suppressor |
| | | | syndrome) | | |
| | | | NM_000546 | | |
| TPD52 | TPD52 | TPD52, D52, | Tumor protein D52 | Oncogene | Cell cycle |
| | (100-185) | N8L, PC-1, | NM_005079 | | |
| | Abnova | PrLZ, hD52 | | | |
| UBE2C | UBE2C | UBE2C, | Ubiquitin- | Cell cycle | Cell cycle |
| | (full- | UBCH10, | conjugating | regulation. | |
| | length) | dJ447F3.2 | enzyme E2C | Required for | |
| | Abnova | | NM_007019 | the destruction | |
| | | | | of mitotic | |
| | | | | cyclins | |
| XLKD1 | XLKD1 | XLKD1, HAR, | Extracellular link | Involved in | Angiogenesis |
| | (full- | LYVE-1, | domain | lymphogenesis | |
| | length) | CRSBP-1 | containing 1 | | |
| | Abnova | | NM_016164 | | |
| ZWINT | ZWINT | ZWINT, | ZW10 interactor | Cell division | Cell Cycle |
| | (full- | KNTC2AP, | NM_001005413 | | |
| | length) | HZwint-1, | | | |
| | Abnova | MGC117174 | | | |

Note:
Numbers in the parenthesis of column 2 indicate the amino acid number for the partial recombinant protein.

Immune profiling of prostate cancer and benign prostatic hyperplasia sera. The protein array chips were blocked with blocking buffer (1×PBS, 1% BSA, 0.1% Tween-20) for 1 hr at 4° C. The blocking was done in a glass staining dish with gentle agitation. For the pooled serum experiment, both PCa and BPH serum pool was made by pooling the sera from the corresponding 32 patients. Then a 16 μl sample from each pool was diluted, 1:150, in probing buffer (1×PBS, 5 mM MgCL2, 0.5 mM DTT, 0.05% Triton-X-100, 5% glycerol, 1% BSA) and used for probing of each array. For individual patient screening, the serum for each patient was diluted, 1:150, in probing buffer and 100 μl of diluted serum was then applied to a low content, protein array. Serum probing was done at 4° C. for 1.5 hours in a moisture chamber (Evergreen, cat#240-9020-Z1 0). After serum probing, the chips were washed three times with the probing buffer (10 min/wash) in a pap jar (1 slide per pap jar in 25 ml volume, Evergreen, cat#222-5450-G8S). The washed chips were then incubated with a goat anti-human IgG secondary antibody labeled with Alexa 647 (Invitrogen, cat#A21445) for 1.5 hours at a 1:2000 dilution in the probing buffer. The chips were washed three times (10 min/wash, in pap jar with 25 ml volume) with probing buffer and then spin dried. The chips were scanned with Axon Genepix 4000B scanner (PMT 600, 33% power). Competition assay was performed in the same way except that each sample was incubated with purified antigens for 30 minutes at 4° C. before they were loaded on the protein array.

Tissue microarray. Prostate cancer tissue array analysis was performed using MaxArray human PCa slides (Invitrogen, cat# 73-5063) with standard protocol. Briefly the tissue microarray slides were twice treated with xylene (Sigma) for 5 minutes. The slides were then treated in absolute ethanol twice for 5 minutes each time, 95% ethanol once for 5 minutes, 80% ethanol once for 5 minutes and finally briefly rinsed with H2O. The rehydrated slides were further treated with Digest-All 4 (Proteinase K) (Invitrogen, cat# 00-3011) for 40 minutes at room temperature in a humid chamber. After blocking with 1% BSA for 1 hour, the slides were probed with a 1:500 dilution of anti-KDR/anti-PIM-1 antibodies to detect KDR or PIM-1 antigens. The KDR antibody was from (Invitrogen) and PIM-1 antibody was from Abcam (ab15002) which was generated against N-terminal peptide amino acid 22-37 (ATKLAPGKEKEPLESQT) of human PIM-1. Both antibodies were labeled with Alexa 647 (Invitrogen). The slides were observed with fluorescence Nikon Eclipse TE 200 (40×) microscope. Images were processed using Image-Pro software (Media Cybernetics). In this experiment, at least 1000 cells from each patient were analyzed and only samples with negative and strong signals were counted.

Data analysis. Data analysis was done by both normalizing (by positive controls on the assay) and not normalizing the data with no statistically difference in the results, so all analysis presented was done with background subtracted unnormalized data.

For both single and the duplex autoantibody assays a logistic regression classifier was fit to the data. Since KDR and PIM-1 are spotted on the array twice, the classifying percentages were average for the pair, and the combined logistic regression (all 4 combinations) were average, results are shown in Table 14.

TABLE 14
Comparison of KDR and PIM-1 predictability for prostate cancer

| | | | | | KDR & |
|---|---|---|---|---|---|
| | PSA | | KDR | PIM-1 | PIM-1 |
| Patient | Measurement | Status | Prediction | Prediction | Prediction |
| 103 | 5.25 | PCa | 99.04% | 82.08% | 99.98% |
| 104 | 3.25 | PCa | 39.80% | 100.00% | 100.00% |
| 105 | 174.84 | PCa | 97.57% | 40.15% | 99.01% |
| 106 | 0.41 | PCa | 40.04% | 95.53% | 97.06% |
| 107 | 4.37 | PCa | 97.41% | 21.55% | 96.75% |
| 109 | 6.95 | PCa | 45.07% | 100.00% | 100.00% |
| 110 | 26.63 | PCa | 53.28% | 49.65% | 51.96% |
| 111 | 8.52 | PCa | 56.47% | 19.54% | 15.05% |
| 112 | 5.77 | PCa | 34.07% | 45.51% | 21.13% |
| 115 | 5.95 | PCa | 17.97% | 90.87% | 69.02% |
| 116 | 4.18 | PCa | 95.16% | 99.94% | 100.00% |
| 119 | 3.71 | PCa | 59.47% | 49.84% | 61.30% |
| 120 | 5.01 | PCa | 97.00% | 75.20% | 99.82% |
| 123 | 1.83 | PCa | 27.73% | 99.98% | 100.00% |
| 126 | 1.35 | PCa | 60.77% | 44.01% | 55.07% |
| 128 | 3.66 | PCa | 32.21% | 37.90% | 12.66% |
| 130 | 8.84 | PCa | 96.66% | 36.84% | 98.15% |
| 131 | 5.05 | PCa | 89.02% | 22.95% | 77.04% |
| 135 | 3.21 | PCa | 89.23% | 70.28% | 98.46% |
| 136 | 2.15 | PCa | 14.74% | 100.00% | 100.00% |
| 137 | 3.96 | PCa | 39.88% | 88.57% | 88.79% |
| 140 | 57.22 | PCa | 98.65% | 30.75% | 99.11% |
| 141 | 4.84 | PCa | 99.81% | 49.14% | 99.98% |
| 142 | 6.8 | PCa | 55.58% | 43.45% | 46.73% |
| 143 | 7.53 | PCa | 36.34% | 69.34% | 55.62% |
| 144 | 11.84 | PCa | 38.61% | 87.09% | 86.21% |
| 150 | 5.13 | PCa | 99.87% | 100.00% | 100.00% |
| 156 | 1.38 | PCa | 40.72% | 53.11% | 38.94% |
| 169 | 9 | PCa | 95.47% | 80.57% | 99.79% |
| 170 | 1.38 | PCa | 95.42% | 23.37% | 93.11% |
| 176 | 2.82 | PCa | 98.34% | 22.98% | 98.34% |
| 177 | 5.1 | PCa | 35.94% | 85.78% | 82.09% |
| 101 | 5.96 | BPH | 35.65% | 25.53% | 7.91% |
| 102 | 30.08 | BPH | 38.50% | 36.80% | 17.37% |
| 108 | 1.93 | BPH | 28.70% | 55.65% | 23.69% |
| 113 | 5.75 | BPH | 16.08% | 26.66% | 1.99% |
| 114 | 4.98 | BPH | 43.09% | 22.55% | 9.73% |
| 117 | 0.9 | BPH | 30.61% | 23.42% | 5.02% |
| 118 | 8.87 | BPH | 24.78% | 23.52% | 3.38% |
| 121 | 21.03 | BPH | 11.37% | 27.28% | 1.20% |
| 122 | 7.01 | BPH | 81.64% | 38.70% | 78.94% |
| 124 | 3.58 | BPH | 13.87% | 60.15% | 9.73% |
| 125 | 3.97 | BPH | 27.87% | 38.68% | 10.53% |
| 127 | 1.42 | BPH | 21.53% | 25.27% | 2.97% |
| 129 | 5.96 | BPH | 26.39% | 25.48% | 4.37% |
| 132 | 1.55 | BPH | 17.07% | 29.28% | 2.63% |
| 133 | 2.45 | BPH | 31.52% | 22.84% | 5.10% |
| 134 | 3.66 | BPH | 40.06% | 42.92% | 24.32% |
| 138 | 3.15 | BPH | 20.72% | 55.83% | 14.45% |
| 139 | 2.47 | BPH | 23.33% | 24.00% | 3.13% |
| 145 | 2.79 | BPH | 59.84% | 26.24% | 27.18% |
| 146 | 0.22 | BPH | 27.14% | 31.67% | 6.83% |
| 147 | 0.61 | BPH | 60.61% | 28.60% | 30.38% |
| 148 | 14.37 | BPH | 45.56% | 74.01% | 72.37% |
| 149 | 6.82 | BPH | 85.73% | 69.76% | 98.24% |
| 152 | 4.56 | BPH | 60.88% | 26.41% | 27.48% |
| 153 | 0.87 | BPH | 14.31% | 28.53% | 1.88% |
| 154 | 3.27 | BPH | 35.98% | 23.28% | 6.87% |
| 155 | 3.24 | BPH | 26.97% | 25.68% | 4.63% |
| 157 | 4.34 | BPH | 32.81% | 43.54% | 18.00% |
| 158 | 6.64 | BPH | 30.05% | 25.42% | 5.57% |
| 160 | 19.43 | BPH | 29.03% | 78.32% | 54.98% |
| 161 | 5.1 | BPH | 32.25% | 76.89% | 57.97% |
| 162 | 7.62 | BPH | 55.15% | 24.52% | 19.39% |

Receiver operator characteristic (ROC) curves were calculated using the average logistic regression for the KDR, PIM-1 and combination of markers. The ROC curve for the PSA assay was calculated using the observed PSA concentrations. The area under the curve (AUC) was calculated for each ROC curve. For each marker or combined markers the optimal point on the ROC curve that maximizes both sensitivity and specificity using the ROC by looking for the maximum of the sum of the sensitivity and specificity, if multiple points on the ROC curve give the same sum of sensitivity and specificity, then the maximum of the squared sum of the tied points was used.

### Results

This Experiment identified multiple prostate cancer tumor associated antigens, and can be extended to any disease that elicits a humoral response. Two novel tumor antigens KDR and PIM-1 were found to diagnose prostate cancer with higher specificity and sensitivity than current clinical PSA test.

Both pooled and individual serum samples were examined in this study. Pooled sera from 32 PCa and pooled sera from 32 BPH were first examined using the high content protein array chip containing all 96 proteins (shown in FIG. 2A, with the position of KDR and PIM-1 indicated). About half of the 96 protein antigens showed detectable autoantibody signals (measured as a minimal fluorescence signal plus three times standard deviation above background level signal). The top 20 biomarkers that had significant difference between pooled PCa sera and pooled BPH are shown in FIG. 2B. Among these proteins, cyclin B1 (CCNB1), alpha-methylacyl-CoA racemase (AMACR) and prostate specific antigen or PSA (KLK3), survivin (BIRC5) have been reported previously to induce autoantibodies in PCa patients. Tumor suppressors P53 (TP53) and mucin1 (MUC1) are two actively studied tumor antigens for various tumors (Casiano et al. (2006) “Tumor-associated antigen arrays for the serological diagnosis of cancer,” Mol Cell Proteomics. 5(10):1745-59; Megliorino et al. (2005) “Autoimmune response to anti-apoptotic protein survivin and its association with antibodies to p53 and c-myc in cancer detection,” Cancer Detect. Prev. 29(3):241-8; and Hirasawa et al. (2000) “Natural autoantibody to MUC1 is a prognostic indicator for non-small cell lung cancer,” Am J Respir Crit Care Med. 161:589-94).

Among the top 20 markers, KDR, PIM-1, LGALS8, GDF15, RPL23, RPL30, SFRP4, QSCN6, NCAM2, HOXB13, SH3GLB1, CLDN3, CLDN4, PTEN are novel tumor antigens that have not been previously reported as inducing an autoantibody response. These markers are involved in various stages of PCa progression, including cell growth and apoptosis (KDR, PIM-1, GDF15, SFRP4, HOXB13, QSCN6), metabolism (RPL23 and RPL30), metastasis and angiogenesis (KDR, PTEN, CLDN3, CLDN4, NCAM2, LGALS8).

KDR and PIM-1 protein spots produced a 2-fold and 1.6-fold higher autoantibody response (respectively) in PCa than in BPH (shown in FIGS. 2A and 2B). On the protein microarray, full-length PIM-1 and a truncated KDR protein (derived from its intracellular domain) were utilized. Both proteins were previously reported to be involved in the development of PCa.

To further characterize and validate the autoantibodies against KDR and PIM-1, a purified protein competition assay was used, as shown in FIG. 2C (KDR) and FIG. 2D (PIM-1). At spiked concentration of 1.8 ug/mL purified KDR added to sera, the KDR autoantibody signal on the protein chip was reduced by 50%. The PIM-1 autoantibody protein chip signal was also reduced by half when purified PIM-1 protein was added to sera at a concentration of 2 μg/mL. The autoantibody signals against other proteins on the same chip were not affected (data not shown). These results suggested that KDR and PIM-1 autoantibody signals are specific to the respective tumor antigens.

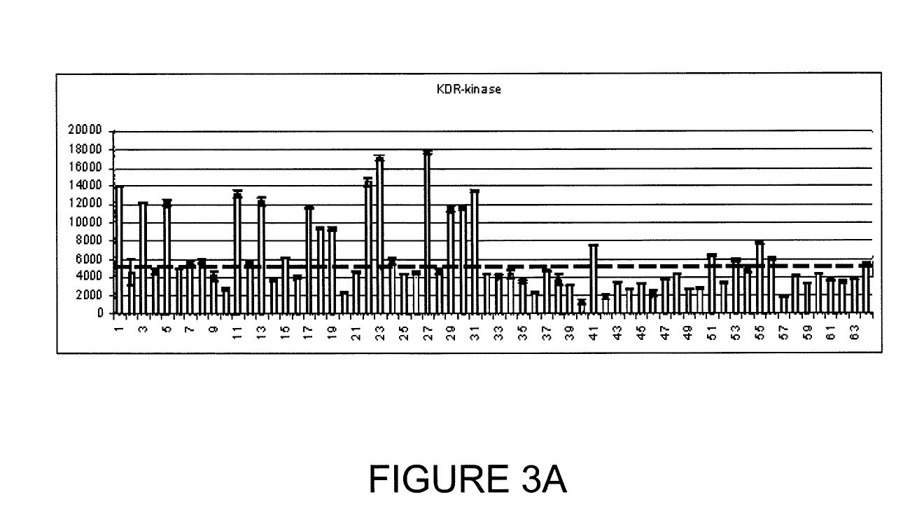

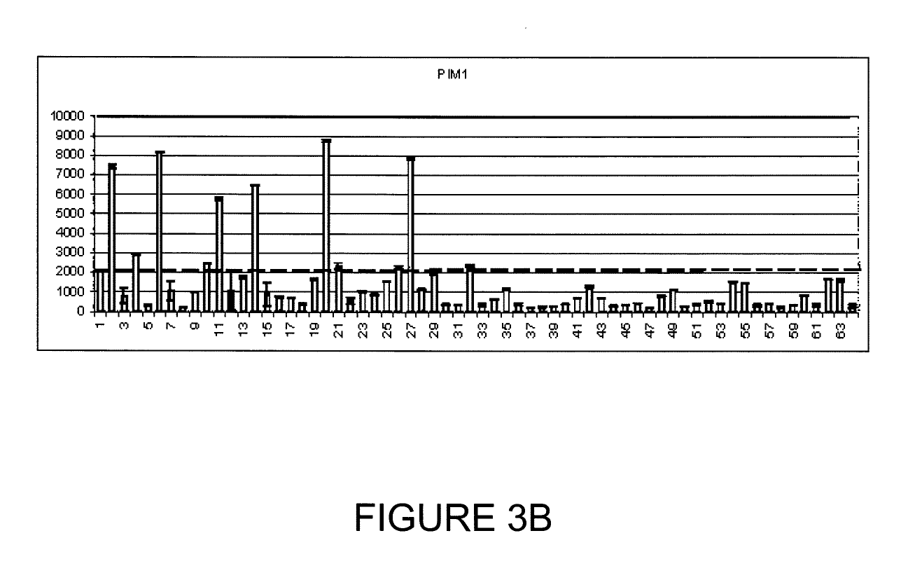

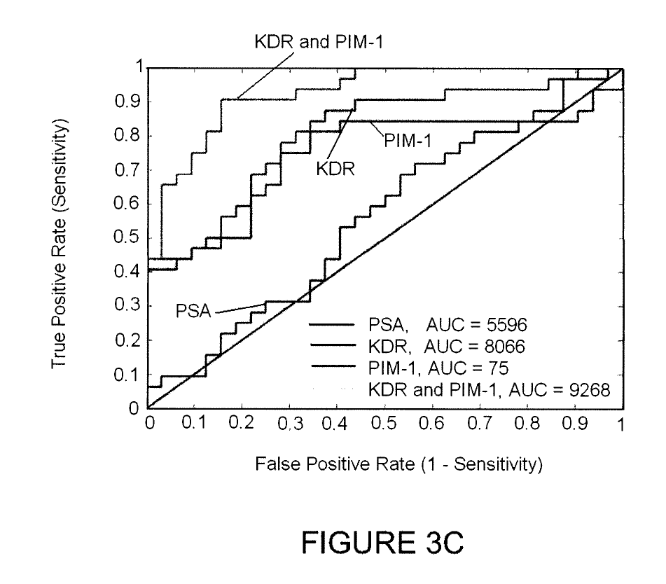

Autoantibodies against KDR and PIM-1 differentiate prostate cancer from benign prostatic hyperplasia. To further investigate KDR and PIM-1 autoantibodies for their combined diagnostic value, a low content chip was made which contains full-length PIM-1 and the intracellular domain of KDR protein printed on 16-pad nitrocellulose slides. In this manner all 64 individual (non-pooled) sera samples can rapidly and reproducibly be measured using a relatively small number of protein array chips (6 individual sera samples per slide, performed in duplicate). The lower-content-protein chip was cross-validated against the full 96-element protein chip with the same serum resulting in CV between the two chips of 8-10% (data not shown). The fluorescence signals for both KDR and PIM-1 are plotted against each other for each patient in FIGS. 3A and 3B. Although KDR gave stronger autoantibody signal than PIM-1, both KDR and PIM-1 showed significantly higher autoantibody signals in PCa than BPH sera. These results are consistent with the immune profiling data using pooled serum samples.

Diagnostic quality of an assay is characterized by using the receiver operating characteristic curve (ROC). The ROC curve for each individual biomarker, the combined biomarkers (PIM1 and KDR) using logistic regression and PSA from 32 PCa versus 32 BPH individually sample sera data set is shown in FIG. 3C. The area under the curve (AUC) for KDR and PIM-1 was 0.8066 and 0.75 respectively, whereas the combined PIM-1 and KDR 2-plex assay gives an AUC of 0.9268, compared to the PSA test with AUC of 0.5596. All combinations of 2-plex and 3-plex assays that included PSA as an additional biomarker were looked at using the ROC (data not shown). Adding PSA did not add any additional diagnostic value in any combination of other markers. As a 1-plex assay, the PSA test yielded 71.9% sensitivity and 43.7% specificity, values similar to previous reports, showing it is not very effective in differentiating PCA from BPH (Etzioni et al. (2002) “Overdiagnosis due to prostate-specific antigen screening: lessons from U.S. prostate cancer incidence trends,” J Natl Cancer Inst. 94(13):981-90).

All PCa and BPH serum samples were biopsy-based classified. Each patient's sera obtained came with a pathology report including: Gleason score, number of positive scores for all 8 needle biopsy cores and a percentage of carcinoma in any single core. Most PCa patients in this study had Gleason scores of 6 (23 out of 32), representing intermediate grade tumors. Detailed examination of the pathology reports from each patient revealed that KDR and PIM-1 gave good differential power in patients with a very low numbers of cancer-positive cores. Patients with either one or two positive cores the KDR and PIM-1 autoantibody assay detects 90% of the cancer cases while PSA detects only 50% of the cancer cases (Table 15). All 11 patients with just a single positive cancerous core (out of the 8 cores biopsied per patient) were correctly categorized using KDR-PIM-1 2-plex assay. Patients with three or more positive cores, both PSA and 2-plex autoantibody assay showed similar sensitivity in diagnosing cancer 83.3% and 91.7% respectively. Positive core numbers correlate with tumor volume (Lewis et al. (2002) “Carcinoma extent in prostate needle biopsy tissue in the prediction of whole gland tumor volume in a screening population,” Am J Clin Pathol. 118(3): 442-450), suggesting that KDR and PIM-1 autoantibodies may be valuable in diagnosing PCa when a tumor may be non-palpable, i.e. not detectable by DRE.

TABLE 15
Correlation of pathology, PSA level and KDR/PIM-1 autoantibody test

| | | Mean of largest | | | | | |
|---|---|---|---|---|---|---|---|
| Number of | Number in | percentage of | | | | PSA | Positive using |
| positive cores | PCa | carcinoma in | Gleason score | | | level 4+ | combined KDR |
| (out of 8) | patients | single core | 6 | 7 | 8+ | ng/ml | and PIM-1 Test |
| 1 | 11 (34%) | 16% | 10/11 | 1/11 | 0 | 5/11 | 11/11 |
| 2 | 9 (28%) | 35% | 7/9 | 2/9 | 0 | 5/9 | 7/9 |
| 3 and above | 12 (38%) | 43% | 6/12 | 2/12 | 4/12 | 10/12 | 11/12 |

KDR and PIM-1 are over-expressed in prostate cancer tissues and over-expression of KDR and PIM-1 correlates with autoantibody frequencies in the patient population. Several mechanisms have been proposed for autoantibody induction in cancer patients, including the gene mutations, over expression, abnormal post-translational modifications, misfolding and aberrant cellular and subcellular localization (Casiano et al. (2006) “Tumor-associated antigen arrays for the serological diagnosis of cancer,” Mol Cell Proteomics. 5(10):1745-59; and Tan, EM. (2001) “Autoantibodies as reporters identifying aberrant cellular mechanisms in tumorigenesis,” J Clin Invest. 108(10):1411-5). Final protein expression level of PIM-1 and KDR were measured to correlate with the autoantibody induction in PCa tissues. An antibody against KDR and PIM-1 respectively were examined for expression patterns of these two proteins in PCa tissues using fluorescence immunohistochemistry.

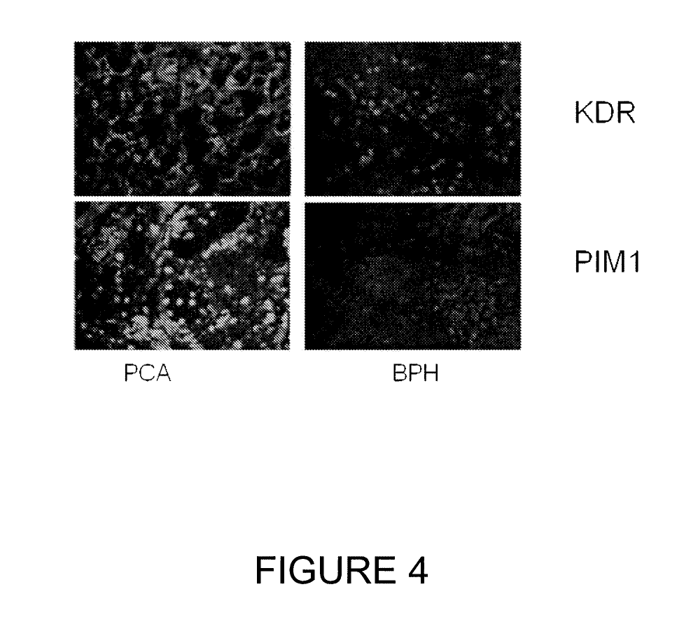

An antibody against the intracellular domain of KDR showed that KDR was localized in the cytoplasm of cancer cells (FIG. 4). Immunostaining with a PIM-1 antibody showed that PIM-1 is localized in both cytoplasm and nucleus (FIG. 4). As shown in Table 16, the KDR-antigen signal was detected in ˜70% of PCa and ˜21% BPH patients on tissue microarrays, indicating KDR is preferentially expressed in PCa tissues. This correlates with the high KDR autoantibody signals in PCa patients (˜62%) versus low KDR autoantibody signals in BPH patients (˜20%). The same trend also observed for PIM-1. PIM-1 expression was detected in ˜30% PCa patients tissue arrays, while almost no BPH patients showed detectable PIM-1 signal in accordance with frequencies of PIM-1 autoantibody in PCa patients (˜37%) and BPH patients (˜0%). In this study only specimens with negative and strong fluorescence signals were used and these results are consistent with previous reports where PIM-1 was detected in 38% cancer cases but not detected in normal controls using the strongest intensity grade (score 3). These results suggest that over expression of KDR and PIM-1 protein, may lead to stronger autoantibody induction in PCa patients.

TABLE 16
KDR and PIM-1 detection in PCa patients and BPH patients

| | | KDR | | PIM-1 |
|---|---|---|---|---|
| | KDR positive | autoantibody | PIM-1 positive | autoantibody |
| | patients | positive | patients by | positive |
| | by tissue | patients on | tissue | patients on |
| | arrays (n = 20) | assay (n = 32) | arrays (n = 20) | assay (n = 32) |
| PCa | 70% | 62% | 30% | 37% |
| BPH | 21% | 20% | 2.6% | 0% |

The percentage of patients with higher autoantibody signals in PCa than in BPH (data not shown) suggests that autoimmune response is more pronounced in PCa than in BPH.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

One of ordinary skill in the art will appreciate that starting materials, reagents, purification methods, materials, substrates, device elements, analytical methods, assay methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

When a group of materials, compositions, components or compounds is disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

All references cited herein are hereby incorporated by reference in their entirety to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional nucleic acids, chemically modified nucleic acids, additional cells, and additional uses of the invention. All headings used herein are for convenience only. All patents and publications mentioned in the specification are indicative of the levels of skill of those skilled in the art to which the invention pertains, and are herein incorporated by reference to the same extent as if each individual publication, patent or patent application was specifically and individually indicated to be incorporated by reference. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein

| Sequence for PIM-1 (Accession number NP_002639) | |
|---|---|
| DEFINITION: | pim-1 oncogene [Homo sapiens]. |
| VERSION | NP_002639.1 GI:4505811 |
| DBSOURCE | REFSEQ: accession NM_002648.2 |
| (SEQ ID NO: 1) | |
| 1 mllskinsla hlraapcndl hatklapgke keplesqyqv gpllgsggfg svysgirvsd | |
| 61 nlpvaikhve kdrisdwgel pngtrvpmev vllkkvssgf sgvirlldwf erpdsfvlil | |
| 121 erpepvqdlf dfitergalq eelarsffwq vleavrhchn cgvlhrdikd enilidlnrg | |
| 181 elklidfgsg allkdtvytd fdgtrvyspp ewiryhryhg rsaavwslgi llydmvcgdi | |
| 241 pfehdeeiir gqvffrqrvs secqhlirwc lalrpsdrpt feeiqnhpwm qdvllpqeta | |
| 301 eihlhslspg psk | |
| Sequence for KDR (Accession number NP_002244) | |
| DEFINITION: | kinase insert domain receptor |
| | (a type III receptor tyrosine kinase) [Homo sapiens]. |
| VERSION | NP_002244.1 GI:11321597 |
| DBSOURCE | REFSEQ: accession NM_002253.1 |

```
(SEQ. ID. NO. 2)
      1 mqskvllava lwlcvetraa svglpsvsld lprlsiqkdi ltikanttlq itcrgqrdld
    61 wlwpnnqsgs eqrvevtecs dglfcktlti pkvigndtga ykcfyretdl asviyvyvqd
  121 yrspfiasvs dqhgvvyite nknktvvipc lgsisnlnvs lcarypekrf vpdgnriswd
  181 skkgftipsy misyagmvfc eakindesyq simyivvvvg yriydvvlsp shgielsvge
  241 klvlnctart elnvgidfnw eypsskhqhk klvnrdlktq sgsemkkfls tltidgvtrs
  301 dqglytcaas sglmtkknst fvrvhekpfv afgsgmeslv eatvgervri pakylgyppp
  361 eikwykngip lesnhtikag hvltimevse rdtgnytvil tnpiskekqs hvvslvvyvp
  421 pqigekslis pvdsyqygtt qtltctvyai ppphhihwyw qleeecanep sqavsvtnpy
  481 pceewrsved fqggnkievn knqfaliegk nktvstlviq aanvsalykc eavnkvgrge
  541 rvisfhvtrg peitlqpdmq pteqesvslw ctadrstfen ltwyklgpqp lpihvgelpt
  601 pvcknldtlw klnatmfsns tndilimelk naslqdqgdy vclaqdrktk krhcvvrqlt
  661 vlervaptit gnlenqttsi gesievscta sgnpppqimw fkdnetlved sgivlkdgnr
  721 nltirrvrke deglyteqac svlgcakvea ffiiegaqek tnleiiilvg taviamffwl
  781 llviilrtvk ranggelktg ylsivmdpde lpldeheerl pydaskwefp rdrlklgkpl
  841 grgafgqvie adafgidkta tcrtvavkml kegathsehr almselkili highhlnvvn
  901 llgactkpgg plmvivefck fgnlstylrs krnefvpykt kgarfrqgkd yvgaipvdlk
  961 rrldsitssq ssassgfvee kslsdveeee apedlykdfl tlehlicysf qvakgmefla
1021 srkcihrdla arnillsekn vvkicdfgla rdiykdpdyv rkgdarlplk wmapetifdr
1081 vytiqsdvws fgvllweifs lgaspypgvk ideefcrrlk egtrmrapdy ttpemyqtml
1141 dcwhgepsqr ptfselvehl gnllqanaqq dgkdyivlpi setlsmeeds glslptspvs
1201 cmeeeevcdp kfhydntagi sqylqnskrk srpvsvktfe dipleepevk vipddnqtds
1261 gmvlaseelk tledrtklsp sfggmvpsks resvasegsn qtsgyqsgyh sddtdttvys
1321 seeaellkli eigvqtgsta qilqpdsgtt lssppv
```

1. A method of detecting an autoantibody in an individual suspected of having prostate cancer, comprising:
a) contacting a sample from the individual with two or more autoantibody capture molecules, wherein the autoantibody capture molecules are autoantibody capture molecules of Table 1 or Table 11a, or target antibodies against an antigen of Table 1 or Table 11a; and
(b) detecting binding of an antibody or antibody-containing complex in the sample to the autoantibody capture molecules, thereby detecting the autoantibody in the individual.

2. The method of claim 1 wherein the autoantibody capture molecules are autoantibody capture molecules of Table 2 or target antibodies against an antigen of Table 2.

3. The method of claim 1 wherein the autoantibody capture molecules are autoantibody capture molecules of Table 3 or target antibodies against an antigen of Table 3.

4. The method of claim 1 wherein the autoantibody capture molecules are autoantibody capture molecules of Table 4 or target antibodies against an antigen of Table 4.

5. The method of claim 1 wherein the autoantibody capture molecules are autoantibody capture molecules of Table 10 or target antibodies against an antigen of Table 10.

6. The method of claim 1 wherein the autoantibody capture molecules are selected from the group consisting of KDR, PIM-1, LGALS8, GDF15, RPL23, RPL30, SFRP4, QSCN6, NCAM2, HOXB13, SH3GLB1, CLDN3, CLDN4, PTEN, CCNB1, AMACR, TP53, MUC1, KLK3, BIRC5, and target antibodies to KDR, PIM-1, LGALS8, GDF15, RPL23, RPL30, SFRP4, QSCN6, NCAM2, HOXB13, SH3GLB1, CLDN3, CLDN4, PTEN, CCNB1, AMACR, TP53, MUC1, KLK3, and BIRC5.

7. The method of claim 1 wherein the autoantibody capture molecules comprise KDR or PIM-1.

8. The method of claim 1 comprising contacting a sample from the individual with an autoantibody detection set of Table 5, Table 6, Table 7, Table 8, or Table 9.

9. The method of claim 1 comprising contacting a sample from the individual with ten or more autoantibody capture molecules, wherein the autoantibody capture molecules are autoantibody capture molecules of Table 1 or Table 11a, or target antibodies against an antigen of Table 1 or Table 11a.

10. The method of claim 1 comprising contacting a sample from the individual with fifty or more autoantibody capture molecules, wherein the autoantibody capture molecules are autoantibody capture molecules of Table 1 or Table 11a, or target antibodies against an antigen of Table 1 or Table 11a.

11. The method of claim 1 wherein the detecting is performed by immunoassay, wherein the autoantibody capture molecules are immobilized on a solid support.

12. The method of claim 1 wherein the test sample is blood or a fraction thereof.

13. The method of claim 1 further comprising correlating the binding of the autoantibody capture molecules to antibodies or antibody-containing complexes in the test sample with a diagnosis of prostate cancer.

14. The method of claim 1 wherein the binding of the autoantibody capture molecules to antibodies or antibody-containing complexes in the test sample distinguishes between prostate cancer and BPH in the individual.

15. The method of claim wherein the binding of the autoantibody capture molecules to antibodies or antibody-containing complexes in the test sample distinguishes between High Grade and Low Grade prostate cancer.

16. A method of detecting an autoantibody in an individual suspected of having prostate cancer, comprising:
a) contacting a sample from the individual with one or more autoantibody capture molecules, wherein the autoantibody capture molecules are selected from the group consisting of KDR, PIM-1, LGALS8, GDF15, RPL23, RPL30, SFRP4, QSCN6, NCAM2, HOXB13, SH3GLB1, CLDN3, CLDN4, PTEN, and target antibodies to KDR, PIM-1, LGALS8, GDF15, RPL23, RPL30, SFRP4, QSCN6, NCAM2, HOXB13, SH3GLB1, CLDN3, CLDN4, and PTEN; and
(b) detecting binding of an antibody or antibody-containing complex in the sample to the one or more autoantibody capture molecules, thereby detecting the autoantibody in the individual.

17. The method of claim 16 wherein the one or more autoantibody capture molecules comprise KDR or PIM-1.

18. A biomarker detection panel for diagnosing, prognosing, monitoring, detecting or staging prostate cancer comprising two or more autoantibody capture molecules, wherein the autoantibody capture molecules are autoantibody capture molecules of Table 1 or Table 11a, or target antibodies against an antigen of Table 1 or Table 11a.

19. The detection panel of claim 18 wherein the autoantibody capture molecules are autoantibody capture molecules of Table 2 or target antibodies against an antigen of Table 2.

20. The detection panel of claim 18 wherein the autoantibody capture molecules are autoantibody capture molecules of Table 3 or target antibodies against an antigen of Table 3.

21. The detection panel of claim 18 wherein the autoantibody capture molecules are autoantibody capture molecules of Table 10 or target antibodies against an antigen of Table 10.

22. The detection panel of claim 18 wherein the autoantibody capture molecules are selected from the group consisting of KDR, PIM-1, LGALS8, GDF15, RPL23, RPL30, SFRP4, QSCN6, NCAM2, HOXB13, SH3GLB1, CLDN3, CLDN4, PTEN, CCNB1, AMACR, TP53, MUC1, KLK3, BIRC5, and target antibodies to KDR, PIM-1, LGALS8, GDF15, RPL23, RPL30, SFRP4, QSCN6, NCAM2, HOXB13, SH3GLB1, CLDN3, CLDN4, PTEN, CCNB1, AMACR, TP53, MUC1, KLK3, and BIRC5.

23. The detection panel of claim 18 wherein the autoantibody capture molecules comprise KDR or PIM-1.

24. The detection panel of claim 18 wherein the autoantibody capture molecules are immobilized on a solid support.

25. The detection panel of claim 18 comprising at least one biomarker detection set of Table 5, Table 6, Table 7, Table 8, or Table 9.

26. The detection panel of claim 18 comprising ten or more autoantibody capture molecules, wherein the autoantibody capture molecules are autoantibody capture molecules of Table 1 or Table 11a, or target antibodies against an antigen of Table 1 or Table 11a.

27. The detection panel of claim 18 comprising fifty or more autoantibody capture molecules, wherein the autoantibody capture molecules are autoantibody capture molecules of Table 1 or Table 11a, or target antibodies against an antigen of Table 1 or Table 11a.

28. The detection panel of claim 18 wherein the biomarker detection panel has an ROC/AUC value for distinguishing the presence of prostate cancer from BPH of 0.780 or greater.

29. A biomarker detection panel for diagnosing, prognosing, monitoring, detecting or staging prostate cancer comprising an autoantibody capture molecule selected from the group consisting of KDR, PIM-1, LGALS8, GDF15, RPL23, RPL30, SFRP4, QSCN6, NCAM2, HOXB13, SH3GLB1, CLDN3, CLDN4, PTEN, fragments thereof comprising an epitope recognizable by an antibody, and target antibodies to KDR, PIM-1, LGALS8, GDF15, RPL23, RPL30, SFRP4, QSCN6, NCAM2, HOXB13, SH3GLB1, CLDN3, CLDN4, and PTEN.

30. The biomarker detection panel of claim 29 comprising KDR, a fragment of KDR comprising an epitope recognizable by an antibody, PIM-1, or a fragment of PIM-1 comprising an epitope recognizable by an antibody.

31. The biomarker detection panel of claim 29 wherein the panel comprises a first biomarker comprising an autoantibody-binding epitope of KDR and a second biomarker comprising an autoantibody-binding epitope of PIM-1.
